(19) United States
(12) Patent Application Publication
(10) Pub. No.: US 2026/0256673 A1
(43) Pub. Date: Sep. 3, 2026

(54) COSMETIC COMPOSITIONS CONTAINING BIODEGRADABLE CELLULOSE ESTER MICROPARTICLES

(71) Applicant: Eastman Chemical Company, Kingsport, TN (US)

(72) Inventors: Hassan Alomgir Ali, Antwerpen (BE); Chloe Beaume, Ghent (BE); Brajesh Jha, Kingsport, TN (US); Matt Stephens, Kingsport, TN (US); Jung Hyun Park, Johnson City, TN (US); Leslie Taylor Baker, Gate City, VA (US); Sharmistha Mazumder, Jonesborough, TN (US); Robert David Pace, Church Hill, TN (US); Kenny Randolph Parker, Afton, TN (US)

(73) Assignee: Eastman Chemical Company, Kingsport, TN (US)

(21) Appl. No.: 19/163,870

(22) PCT Filed: Mar. 22, 2024
(86) PCT No.: PCT/US2024/021025

§ 371 Date: Sep. 10, 2025

Related U.S. Application Data
(60) Provisional application No. 63/491,623, filed on Mar. 22, 2023.

(51) Int. Cl. A61K 8/73; A61K 8/02; A61K 8/36; A61Q 1/02; A61Q 1/06; A61Q 17/04; A61Q 19/00

(52) U.S. Cl. CPC A61K 8/731; A61K 8/0241; A61K 8/36; A61Q 1/02; A61Q 1/06; A61Q 17/04; A61Q 19/007; A61K 2800/594; A61K 2800/654

(57) ABSTRACT

Cosmetic compositions comprising biodegradable microparticles formed from biodegradable mixed cellulose esters are provided. More particularly, cosmetic compositions are provided that contain environmentally friendly cellulose-based microparticles, which can be used in a wide array of cosmetic and personal care applications, and provide enhanced cosmetic attributes.

### BACKGROUND

Microparticles are particles that are less than 1 millimeter (mm) in diameter. These particles are sometimes included in consumer products, such as personal care and cosmetic products. Many of these microparticle-containing products are designed to be applied and then washed or rinsed from the user's body. When the microparticle-containing products are washed or rinsed from the user's body, the particles are flushed down the drain and received at municipal water treatment facilities. In the past, many known microparticles had been formed from plastic or polymeric materials, such as polyethylene, polypropylene, polymethyl methacrylate, nylon, polyurethane, and the like. These materials generally have limited biodegradability. In addition, the small size of the particles limits their ability to be captured at the water treatment facilities, such that the particles may be discharged from the facilities and into larger bodies of water (e.g., rivers, seas, and oceans). Once in these larger bodies of water, the plastic or polymeric microparticles may be ingested by wildlife or cause other environmental concerns. Thus, the possibility of producing microparticle particles from more environmentally friendly materials has recently been explored. However, consumers tend to have high expectations when it comes to the personal care and/or cosmetic products they use, including those containing microparticles.

Thus, it is desirable to develop commercially desirable cosmetic compositions containing biodegradable microparticles that meet the high expectations of everyday consumers.

### SUMMARY

In one aspect, the present technology concerns a cosmetic composition comprising biodegradable microparticles, wherein the biodegradable microparticles comprise a mixed cellulose ester. The biodegradable microparticles also exhibit at least 50 percent biodegradability at 60 days according to at least one of the OECD 301B, OECD 301C, or OECD 301F test methods. Furthermore, the mixed cellulose ester comprises: (a) an average degree of substitution for acetyl substituents (“DS_{Ac}”) in the range of 0.1 to 2.3, (b) an average degree of substitution for propionyl substituents (“DS_{Pr}”) or an average degree of substitution for butyryl substituents (“DS_{Bu}”) in the range of 0.1 to 1.5, and (c) an average degree of substitution for hydroxyl substituents (“DS_{OH}”) in the range of 0.6 to 2.8.

In one aspect, the present technology concerns a cosmetic composition comprising 0.5 to 15 weight percent of biodegradable microparticles, wherein the biodegradable microparticles comprise a mixed cellulose ester. In embodiments, the biodegradable microparticles also exhibit at least 50 percent biodegradability at 60 days according to at least one of the OECD 301B, OECD 301C, or OECD 301F test methods and have an average BET surface area in the range of 0.1 to 100, or 1 to 20 m²/g. Furthermore, in embodiments, the mixed cellulose ester comprises: (a) an average degree of substitution for acetyl substituents (“DS_{Ac}”) in the range of 0.1 to 2.3, or 1.5 to 2.3 (b) an average degree of substitution for butyryl substituents (“DS_{Bu}”) in the range of 0.1 to 1.5, or 0.1 to 0.3 and (c) an average degree of substitution for hydroxyl substituents (“DS_{OH}”) in the range of 0.6 to 2.8, or 0.6 to 1.0.

In one aspect, the present technology concerns a process for forming a cosmetic composition comprising biodegradable microparticles. Generally, the process comprises: (a) providing a plurality of biodegradable microparticles comprising a mixed cellulose ester, wherein the biodegradable microparticles exhibit at least 50 percent biodegradability at 60 days according to at least one of the OECD 301B, OECD 301C, or OECD 301F test methods; (b) combining the biodegradable microparticles with one or more cosmetic additives to thereby form a pre-cosmetic mixture; and (c) forming the cosmetic composition from the pre-cosmetic mixture. Furthermore, the mixed cellulose ester comprises: (a) an average degree of substitution for acetyl substituents (“DS_{Ac}”) in the range of 0.1 to 2.3, (b) an average degree of substitution for propionyl substituents (“DS_{Pr}”) or an average degree of substitution for butyryl substituents (“DS_{Bu}”) in the range of 0.1 to 1.5, and (c) an average degree of substitution for hydroxyl substituents (“DS_{OH}”) in the range of 0.6 to 2.8.

### BRIEF DESCRIPTION OF THE FIGURES

FIG. 1 is a schematic diagram of an example process for making cellulose ester microparticles.

FIG. 2 is a more detailed schematic diagram of an example process for making cellulose ester microparticles

FIG. 3 is a schematic diagram of an alternative process for making cellulose ester microparticles by serialized particle formation.

FIG. 4 is a schematic diagram of an alternative process for making cellulose ester microparticles by separated dope and aqueous mixture formation, and combined emulsion/dispersion formation and particle hardening.

FIG. 5 is a schematic diagram of an alternative process for making cellulose ester microparticles by combined emulsion/dispersion formation.

FIG. 6 is a schematic diagram of an alternative process for making cellulose ester microparticles by combined emulsion/dispersion formation and particle hardening.

FIG. 7 is a schematic diagram of an alternative process for making cellulose ester microparticles by solvent flashing prior to particle hardening.

### DETAILED DESCRIPTION

The present disclosure is directed to cosmetic compositions formed with biodegradable cellulose ester microparticles that exhibit superior properties relative to conventional microparticles. The processes disclosed herein enable the solidity of the produced microparticles to be controlled such that microparticles with desirable properties may be obtained. Alternatively, microparticles produced by mechanical micronization or size reduction from the (as produced) bulk cellulose esters can also provide CE microparticles with desirable properties. These qualities, along with the biodegradability of CE, make the CE microparticles produced herein desirable for use in cosmetic compositions.

The present invention may be understood more readily by reference to the following detailed description and the examples provided therein. It is to be understood that this disclosure is not limited to the specific methods, formulations, and conditions described, as such may vary. It is also to be understood that the terminology used herein is for the purpose of describing particular aspects of the disclosed embodiments only and is not intended to be limiting.

Values may be expressed as “about” or “approximately” a given number. Similarly, ranges may be expressed herein as from “about” one particular value and/or to “about” or another particular value. When such a range is expressed, another aspect includes from the one particular value and/or to the other particular value. Similarly, when values are expressed as approximations, by use of the antecedent “about,” it will be understood that the particular value forms another aspect.

As used herein, the terms “a,” “an,” and “the” mean one or more.

As used herein, the term “and/or,” when used in a list of two or more items, means that any one of the listed items can be employed by itself or any combination of two or more of the listed items can be employed. For example, if a composition is described as containing components A, B, and/or C, the composition can contain A alone; B alone; C alone; A and B in combination; A and C in combination, B and C in combination; or A, B, and C in combination.

As used herein, the terms “comprising,” “comprises,” and “comprise” are open-ended transition terms used to transition from a subject recited before the term to one or more elements recited after the term, where the element or elements listed after the transition term are not necessarily the only elements that make up the subject.

As used herein, the terms “having,” “has,” and “have” have the same open-ended meaning as “comprising,” “comprises,” and “comprise” provided above.

As used herein, the terms “including,” “includes,” and “include” have the same open-ended meaning as “comprising,” “comprises,” and “comprise” provided above.

As used herein, a “mixed cellulose ester” shall denote a cellulose ester having at least two different ester substituents on a single cellulose ester polymer chain.

“Degree of Substitution” is used to describe the average substitution level of the substituents per anhydroglucose unit (“AGU”). Generally, conventional cellulose contains three hydroxyl groups in each AGU that can be substituted. Therefore, the DS can have a value between 0 and 3. However, low molecular weight cellulose mixed esters can have a total degree of substitution slightly above 3 from end group contributions. Low molecular weight cellulose mixed esters are discussed in more detail subsequently in this disclosure. Because DS is a statistical mean value, a value of 1 does not assure that every AGU has a single substituent. In some cases, there can be unsubstituted anhydroglucose units, some with two and some with three substituents, and more often than not the value will be a noninteger. Total DS is defined as the average number of all of substituents per anhydroglucose unit. The degree of substitution per AGU can also refer to a particular substituent, such as, for example, hydroxyl, acetyl, butyryl, or propionyl. Additionally, the degree of substitution can specify a given hydroxyl based on the carbon unit of the anhydroglucose unit.

When the degree of substitution refers to hydroxyl, i.e., DS_{OH}, the reference is to the average hydroxyl groups per anhydroglucose that are not substituted. As a result, DS_{OH} is not used in the calculation of the total degree of substitution.

The present description uses numerical ranges to quantify certain parameters relating to the invention. It should be understood that when numerical ranges are provided, such ranges are to be construed as providing literal support for claim limitations that only recite the lower value of the range as well as claim limitations that only recite the upper value of the range. For example, a disclosed numerical range of 10 to 100 provides literal support for a claim reciting “greater than 10” (with no upper bounds) and a claim reciting “less than 100” (with no lower bounds).

The present description uses specific numerical values to quantify certain parameters relating to the invention, where the specific numerical values are not expressly part of a numerical range. It should be understood that each specific numerical value provided herein is to be construed as providing literal support for a broad, intermediate, and narrow range. The broad range associated with each specific numerical value is the numerical value plus and minus 60 percent of the numerical value, rounded to two significant digits. The intermediate range associated with each specific numerical value is the numerical value plus and minus 30 percent of the numerical value, rounded to two significant digits. The narrow range associated with each specific numerical value is the numerical value plus and minus 15 percent of the numerical value, rounded to two significant digits. For example, if the specification describes a specific temperature of 62° F., such a description provides literal support for a broad numerical range of 25° F. to 99° F. (62° F.+/−37° F.), an intermediate numerical range of 43° F. to 81° F. (62 □F+/−19° F.), and a narrow numerical range of 53° F. to 71° F. (62° F.+/−9° F.). These broad, intermediate, and narrow numerical ranges should be applied not only to the specific values, but should also be applied to differences between these specific values. Thus, if the specification describes a first pressure of 110 psia and a second pressure of 48 psia (a difference of 62 psi), the broad, intermediate, and narrow ranges for the pressure difference between these two streams would be 25 to 99 psi, 43 to 81 psi, and 53 to 71 psi, respectively.

Throughout this application, where patents or publications are referenced, the disclosures of these references in their entireties are intended to be incorporated by reference into this application, to the extent they are not inconsistent with the present invention, in order to more fully describe the state of the art to which the invention pertains.

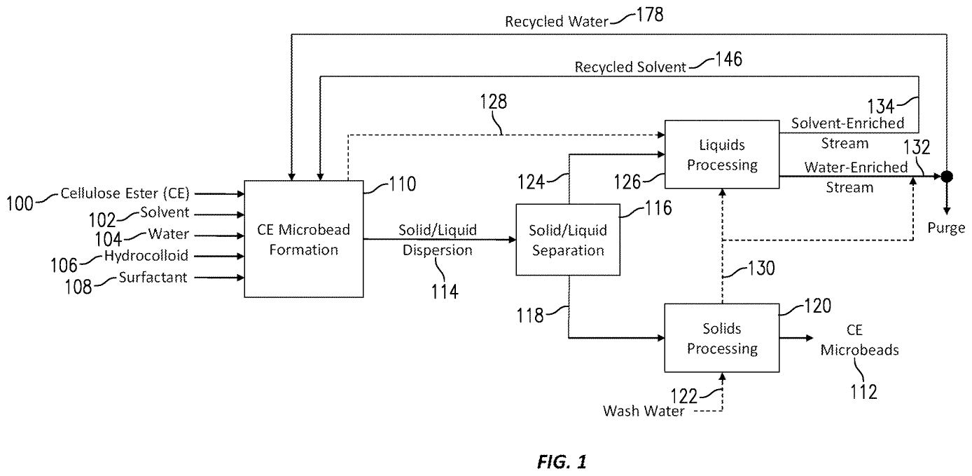

In certain aspects, CE microparticles can be produced via a solvent or emulsion process. Referring now to FIG. 1, cellulose ester (CE) 100, solvent 102, water 104, hydrocolloid 106, and surfactant 108 may be combined in one or more units to make CE microparticles 112. In the illustrated example, CE 100, solvent 102, water 104, hydrocolloid 106, and surfactant 108 are combined at unit 110 to form an initial emulsion therein. The initial emulsion includes a dispersed phase and a continuous phase. The dispersed phase includes at least a portion of CE 100 and at least a portion of solvent 102. The continuous phase includes at least a portion of water 104, at least a portion of hydrocolloid 106, and at least a portion of surfactant 108.

Once combined, the initial emulsion may be agitated at unit 110 to form a pre-hardened dispersion including a sold phase and a liquid phase. The solid phase includes initial microparticles including at least a portion of CE 100 and at least a portion of solvent 102. The liquid phase includes at least a portion of water 104, at least a portion of hydrocolloid 106, and at least a portion of surfactant 108. Additional water 104 (i.e., a drowning liquid/extractant) may be added to the pre-hardened dispersion to increase the concentration of water around the initial microparticles. An initial hardening dispersion including the initial microparticles and the drowning liquid is formed therefrom. The initial hardening dispersion may be agitated at unit 110 to promote the transfer of solvent from the initial microparticles to the drowning liquid. This solvent transfer facilitates hardening the initial microparticles to produce hardened CE microparticles 112, which are entrained in a solvent-ladened drowning liquid.

CE microparticles 112 may be recovered from this dispersion 114 first by processing at a solid/liquid separation unit 116. A solid stream 118 including CE microparticles 112 may be channeled to a solids processing unit 120, where CE microparticles 112 are washed with water 122 and then dried to recover CE microparticles 112, as will be described in more detail below. A liquid stream 124 formed from the solvent-ladened drowning liquid may be channeled to a liquids processing unit 126. Optionally, an excess liquid stream 128 channeled from unit 110 and a wash water stream 130 from unit 120 may also be received at unit 126 for processing therein.

At unit 126, the liquids received therein may be separated into at least a water-enriched stream 132 and a solvent-enriched stream 134. In some embodiments, water-enriched stream 132 may be recovered and at least a portion of which utilized in unit 110, for example. In addition, solvent-enriched stream 134 may be recycled and utilized as at least a portion of the solvent for forming CE microparticles 112 in unit 110. Re-utilization of the recovered water and/or solvent facilitates improving the economics of the CE microparticle formation processes described herein.

In certain aspects, CE microparticles can be produced from mixed cellulose esters (e.g., cellulose acetate butyrate CAB) by subjecting a bulk cellulose ester (CE) form to a mechanical micronization or size reduction process. In embodiments, the mechanical micronization or size reduction process is a pulverization process that uses one of more high-speed gas or liquid jets to pulverize and reduce the size of the bulk CE material. In embodiments, the pulverization process is a jet milling process that uses one or more high-speed gas jets (e.g., air or inert gas) to reduce the size of the bulk CE material. In embodiments, the jet milling process utilizes a fluidized bed opposed jet mill. In embodiments, the mechanical size reduction process provides CE microparticles having an average sphericity of less than 60, or less than 50, or less than 40, or less than 30 percent. In embodiments, the mechanical size reduction process provides CE microparticles having a D[4,3] mean particle size less than 40, or less than 30, or less than 20 microns.

### Mixed Cellulose Esters

In one embodiment or in combination with any embodiment mentioned herein, the CE 100 can be a mixed cellulose ester.

Generally, the cellulose esters described herein, such as CE 100, can be produced by any method known in the art. Examples of processes for producing cellulose esters are taught in Kirk-Othmer, Encyclopedia of Chemical Technology, 5th Edition, Vol. 5, Wiley-Interscience, New York (2004), pp. 394-444, the disclosure of which is incorporated by reference in its entirety. Cellulose, the starting material for producing cellulose esters, can be obtained in different grades and from sources such as, for example, lignocellulosic sources (e.g., softwood pulp, hardwood pulp), cotton linters, corn fiber and other agricultural sources, and bacterial celluloses.

One method of producing cellulose esters is by esterification. In such a method, the cellulose is mixed with the appropriate organic acids, acid anhydrides, and/or catalysts and then converted to a cellulose triester. Ester hydrolysis is then performed by adding a water-acid mixture to the cellulose triester, which can be filtered to remove any gel particles or fibers. Water is added to the mixture to precipitate out the cellulose ester. The cellulose ester can then be washed with water to remove reaction by-products followed by dewatering and drying.

Acylating reagents suitable for use herein can include, but are not limited to, alkyl or aryl carboxylic anhydrides, carboxylic acid halides, and/or carboxylic acid esters containing the above-described alkyl or aryl groups suitable for use in the acyl substituents of the substituted cellulose esters described herein. Examples of suitable carboxylic anhydrides include, but are not limited to, acetic anhydride, propionic anhydride, butyric anhydride, pivaloyl anhydride, benzoic anhydride, and naphthoyl anhydride. Examples of carboxylic acid halides include, but are not limited to, acetyl, propionyl, butyryl, pivaloyl, benzoyl, and naphthoyl chlorides or bromides. Examples of carboxylic acid esters include, but are not limited to, acetyl, propionyl, butyryl, pivaloyl, benzoyl and naphthoyl methyl esters. In one or more embodiments, the acylating reagent can be one or more carboxylic anhydrides selected from the group consisting of acetic anhydride, propionic anhydride, butyric anhydride, pivaloyl anhydride, benzoyl anhydride, and naphthoyl anhydride.

In various embodiments, the cellulose triesters that are hydrolyzed can have three substituents selected independently from alkanoyls having from 2 to 12 carbon atoms. Examples of cellulose triesters include cellulose triacetate, cellulose tripropionate, cellulose tributyrate, or mixed triesters of cellulose, such as cellulose acetate propionate and cellulose acetate butyrate. These cellulose triesters can be prepared by a number of methods known to those skilled in the art. For example, cellulose triesters can be prepared by heterogeneous acylation of cellulose in a mixture of carboxylic acid and anhydride in the presence of a catalyst, such as H₂SO₄. Cellulose triesters can also be prepared by the homogeneous acylation of cellulose dissolved in an appropriate solvent such as LiCl/DMAc or LiCl/NMP.

After esterification of the cellulose to the triester, part of the acyl substituents can be removed by hydrolysis or by alcoholysis to give a secondary cellulose ester. Secondary cellulose esters can also be prepared directly with no hydrolysis by using a limiting amount of acylating reagent. This process is particularly useful when the reaction is conducted in a solvent that will dissolve cellulose.

The cellulose esters thus prepared generally comprise the following structure:

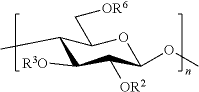

where R², R³, and R⁶ are hydrogen (with the proviso that R², R³, and R⁶ are not hydrogen simultaneously), alkyl-acyl groups, and/or aryl-acyl groups (such as those described above) bound to the cellulose via an ester linkage.

The degree of polymerization (“DP”) of the cellulose esters prepared by these methods can be at least 10. In other embodiments, the DP of the cellulose esters can be at least 50, at least 100, or at least 250. In other embodiments, the DP of the cellulose esters can be in the range of from about 5 to about 100, or in the range of from about 10 to about 50.

Acylating reagents suitable for use herein can include, but are not limited to, alkyl or aryl carboxylic anhydrides, carboxylic acid halides, and/or carboxylic acid esters containing the above-described alkyl or aryl groups suitable for use in the acyl substituents of the substituted cellulose esters described herein. Examples of suitable carboxylic anhydrides include, but are not limited to, acetic anhydride, propionic anhydride, butyric anhydride, pivaloyl anhydride, benzoic anhydride, and naphthoyl anhydride. Examples of carboxylic acid halides include, but are not limited to, acetyl, propionyl, butyryl, pivaloyl, benzoyl, and naphthoyl chlorides or bromides. Examples of carboxylic acid esters include, but are not limited to, acetyl, propionyl, butyryl, pivaloyl, benzoyl and naphthoyl methyl esters. In one or more embodiments, the acylating reagent can be one or more carboxylic anhydrides selected from the group consisting of acetic anhydride, propionic anhydride, butyric anhydride, pivaloyl anhydride, benzoyl anhydride, and naphthoyl anhydride.

The present application discloses, in a first aspect, a mixed ester cellulose ester (“MCE”), comprising: (1) a plurality of acetyl substituents; (2) a plurality of propionyl substituents; and (3) a plurality of hydroxyl substituents, wherein: the MCE has an average degree of substitution for the acetyl substituents (“DS_{Ac}”) is from 0.1 to 2.3, the MCE has an average degree of substitution for the propionyl substituents (“DS_{Pr}”) is from 0.1 to 1.5, the MCE has an average degree of substitution for the hydroxyl substituents (“DS_{OH}”) is from 0.6 to 2.8.

In one embodiment or in combination with any other embodiment, class or subclass of this first aspect, wherein the DS_{Ac} is at least 0.1, at least 0.2, at least 0.3, at least 0.4, at least 0.5, at least 0.6, at least 0.7, at least 0.8, at least 0.9, at least 1.0, at least 1.1, at least 1.2, at least 1.3, at least 1.4, at least 1.5, at least 1.6, at least 1.7, at least 1.8, at least 1.9, or at least 2.0. Additionally, or in the alternative, the DS_{Ac} is less than 2.3, less than 2.2, less than 2.1, less than 2.0, less than 1.9, less than 1.8, less than 1.7, less than 1.6, less than 1.5, less than 1.4, less than 1.3, less than 1.2, less than 1.1, less than 1.0, less than 0.9, less than 0.8, less than 0.7, less than 0.5, less than 0.4, or less than 0.3.

In one embodiment or in combination with any other embodiment, class or subclass of this first aspect, wherein the DS_{Ac} is from 0.6 to 2.2, or 0.6 to 2.1, or 0.6 to 2.0, or 0.6 to 1.9, or 0.6 to 1.8, or 0.7 to 2.3, or 0.7 to 2.2, or 0.7 to 2.1, or 0.7 to 2.0, or 0.7 to 1.9, or 0.8 to 2.3, or 0.8 to 2.2, or 0.8 to 2.1, or 0.8 to 2.0, or 0.8 to 1.9, or 0.9 to 2.3, or 0.9 to 2.2, or 0.9 to 2.1, or 0.9 to 2.0, or 0.9 to 1.9, or 1.0 to 2.3 or 1.0 to 2.2, or 1.0 to 2.1, or 1.0 to 2.0, or 1.0 to 1.9, or 1.1 to 2.3, or 1.1 to 2.2, or 1.1 to 2.1, or 1.1 to 2.0, or 1.1 to 1.9, or 1.2 to 2.3 or 1.2 to 2.2, or 1.2 to 2.1, or 1.2 to 2.0, or 1.2 to 1.9, or 0.6 to 1.5, or 0.6 to 1.3, or 0.6 to 1.1, or 0.6 to 0.9 or 0.7 to 1.5, or 0.7 to 1.3, or 0.7 to 1.1, or 0.7 to 0.9.

In one embodiment or in combination with any other embodiment, class or subclass of this first aspect, wherein the DS_{Pr} is at least 0.05, at least 0.1, at least 0.15, at least 0.2, at least 0.3, at least 0.4, at least 0.5, at least 0.6, at least 0.7, at least 0.8, at least 0.9, at least 1.0, at least 1.1, at least 1.2, at least 1.3, or at least 1.4. Additionally, or in the alternative, the DS_{Pr} is less than 1.5, less than 1.4, less than 1.3, less than 1.2, less than 1.1, less than 1.0, less than 0.9, less than 0.8, less than 0.7, less than 0.5, less than 0.4, less than 0.35, less than 0.3, or less than 0.25.

In one embodiment or in combination with any other embodiment, class or subclass of this first aspect, wherein the DS_{Pr} is from 0.05 to 0.9, or 0.05 to 0.85, or 0.05 to 0.8, or 0.05 to 0.75, or 0.05 to 0.7, or 0.05 to 0.6, or 0.05 to 0.5, or 0.05 to 0.4, or 0.05 to 0.35, or 0.05 to 0.3, or 0.05 to 0.25, or 0.1 to 0.9, or 0.1 to 0.85, or 0.1 to 0.8, or 0.1 to 0.75, or 0.1 to 0.7, or 0.1 to 0.6, or 0.1 to 0.5, or 0.1 to 0.4, or 0.1 to 0.35, or 0.1 to 0.3, or 0.1 to 0.25, or 0.15 to 0.95, or 0.15 to 0.9, or 0.15 to 0.85, or 0.15 to 0.8, or 0.15 to 0.75, or 0.15 to 0.7, or 0.15 to 0.65, or 0.15 to 0.6, or 0.15 to 0.5, or 0.15 to 0.4, or 0.15 to 0.35, or 0.15 to 0.3, or 0.15 to 0.25, or 0.2 to 0.95, or 0.2 to 0.9, or 0.2 to 0.85, or 0.2 to 0.8, or 0.2 to 0.75, or 0.2 to 0.7, or 0.2 to 0.65, 0.25 to 0.95, or 0.25 to 0.9, or 0.25 to 0.85, or 0.25 to 0.8, or 0.25 to 0.75, or 0.25 to 0.7, or 0.25 to 0.65, or 0.3 to 0.95, or 0.3 to 0.9, or 0.3 to 0.85, or 0.3 to 0.8, or 0.3 to 0.75, or 0.3 to 0.7, or 0.3 to 0.65, or 0.35 to 0.95, or 0.35 to 0.9, or 0.35 to 0.85, or 0.35 to 0.8, or 0.35 to 0.75, or 0.35 to 0.7, or 0.35 to 0.65, or 0.4 to 0.95, or 0.4 to 0.9, or 0.4 to 0.85, or 0.4 to 0.8, or 0.4 to 0.75, or 0.4 to 0.7, or 0.4 to 0.65, or 0.45 to 0.95, or 0.45 to 0.9, or 0.45 to 0.85, or 0.45 to 0.8, or 0.45 to 0.75, or 0.45 to 0.7, or 0.45 to 0.65, or 0.5 to 0.95, or 0.5 to 0.9, or 0.5 to 0.85, or 0.5 to 0.8, or 0.5 to 0.75, or 0.5 to 0.7, or 0.5 to 0.65, or 0.1 to 0.9, or 0.1 to 0.85, or 0.1 to 0.8.

In one embodiment or in combination with any other embodiment, class or subclass of this first aspect, wherein the DS_{OH} is at least 0.5, at least 0.55, at least 0.6, at least 0.65, at least 0.7, at least 0.75, at least 0.8, at least 0.85, at least 0.9, at least 1.0, at least 1.1, at least 1.2, at least 1.3, at least 1.4, at least 1.5, at least 1.6, at least 1.7, at least 1.8, at least 1.9, at least 2.0, at least 2.1, at least 2.2, at least 2.3, at least 2.4, at least 2.5, or at least 2.6. Additionally, or in the alternative, the DS_{OH} is less than 2.8, less than 2.7, less than 2.6, less than 2.5, less than 2.4, less than 2.3, less than 2.2, less than 2.1, less than 2.0, less than 1.9, less than 1.8, less than 1.7, less than 1.6, less than 1.5, less than 1.4, less than 1.3, less than 1.2, less than 1.15, less than 1.1, less than 1.05, less than 1.0, less than 0.95, less than 0.9, less than 0.85, or less than 0.8.

In one embodiment or in combination with any other embodiment, class or subclass of this first aspect, wherein the DS_{OH} is from 0.5 to 1.5, or 0.5 to 1.45, or 0.5 to 1.40, or 0.5 to 1.35, or 0.5 to 1.30, or 0.5 to 1.25, or 0.5 to 1.2, or 0.5 to 1.15, or 0.5 to 1.1, or 0.5 to 1.05, or 0.5 to 1.0, or 0.5 to 0.95, or 0.5 to 0.9, or 0.5 to 0.85, or 0.5 to 0.8, or 0.55 to 1.5, or 0.55 to 1.45, or 0.55 to 1.40, or 0.55 to 1.35, or 0.55 to 1.30, or 0.55 to 1.25, or 0.55 to 1.2, or 0.55 to 1.15, or 0.55 to 1.1, or 0.55 to 1.05, or 0.55 to 1.0, or 0.55 to 0.95 or 0.55 to 0.9, or 0.55 to 0.85, or 0.55 to 0.8, or 0.6 to 1.5, or 0.6 to 1.45, or 0.6 to 1.40, or 0.6 to 1.35, or 0.6 to 1.30, or 0.6 to 1.25, or 0.6 to 1.2, or 0.6 to 1.15, or 0.6 to 1.1, or 0.6 to 1.05, or 0.6 to 1.0, or 0.6 to 0.95 or 0.6 to 0.9, or 0.6 to 0.85, or 0.6 to 0.8, or 0.65 to 1.5, or 0.65 to 1.45, or 0.65 to 1.40, or 0.65 to 1.35, or 0.65 to 1.30, or 0.65 to 1.25, or 0.65 to 1.2, or 0.65 to 1.15, or 0.65 to 1.1, or 0.65 to 1.05, or 0.65 to 1.0, or 0.65 to 0.95, or 0.65 to 0.9, or 0.65 to 0.85, or 0.65 to 0.8, or 0.7 to 1.5, or 0.7 to 1.45, or 0.7 to 1.40, or 0.7 to 1.35, or 0.7 to 1.30, or 0.7 to 1.25, or 0.7 to 1.2, or 0.7 to 1.15, or 0.7 to 1.1, or 0.7 to 1.05, or 0.7 to 1.0, or 0.7 to 0.95, or 0.7 to 0.9, or 0.7 to 0.85, or 0.7 to 0.8, or 0.75 to 1.5, or 0.75 to 1.45, or 0.75 to 1.40, or 0.75 to 1.35, or 0.75 to 1.30, or 0.75 to 1.25, or 0.75 to 1.2, or 0.75 to 1.15, or 0.75 to 1.1, or 0.75 to 1.05, or 0.75 to 1.0, or 0.75 to 0.95 or 0.75 to 0.9, or 0.8 to 1.5, or 0.8 to 1.45, or 0.8 to 1.40, or 0.8 to 1.35, or 0.8 to 1.30, or 0.8 to 1.25, or 0.8 to 1.2, or 0.8 to 1.15, or 0.8 to 1.1, or 0.8 to 1.05, or 0.8 to 1.0, or 0.8 to 0.95 or 0.8 to 0.9, or 0.85 to 1.5, or 0.85 to 1.45, or 0.85 to 1.40, or 0.85 to 1.35, or 0.85 to 1.30, or 0.85 to 1.25, or 0.85 to 1.2, or 0.85 to 1.15, or 0.85 to 1.1, or 0.85 to 1.05, or 0.85 to 1.0, or 0.85 to 0.95 or 0.85 to 0.9.

In one embodiment or in combination with any other embodiment, class or subclass of this first aspect, wherein the sum of DS_{Pr} and DS_{Ac} is from 1.9 to 2.44, or 1.9 to 2.0, or 1.9 to 2.1, or 1.9 to 2.2, or 1.9 to 2.3, or 2.0 to 2.44, or 2.0 to 2.1, or 2.0 to 2.2, or 2.0 to 2.3, or 2.1 to 2.44, or 2.1 to 2.2, or 2.1 to 2.3, or 2.2 to 2.44, or 2.2 to 2.3.

In one embodiment or in combination with any other embodiment, class or subclass of this first aspect, wherein the mixed cellulose ester has a ratio of hydroxyl substituents to acetyl substituents of at least 0.4:1, at least 0.45:1, at least 0.5:1, at least 0.6:1, at least 0.7:1, at least 0.8:1, at least 0.9:1, at least 1:1, at least 1.1:1, at least 1.2:1, at least 1.3:1, at least 1.4:1, at least 1.5:1, at least 1.6:1, at least 1.7:1, at least 1.8:1, at least 1.9:2, or at least 2:1. Additionally, in the alternative, the mixed cellulose ester has a ratio of hydroxyl substituents to acetyl substituents of less than 2:1, less than 1.9:1, less than 1.8:1, less than 1.7:1, less than 1.6:1, less than 1.5:1, less than 1.4:1, less than 1.3:1, less than 1.2:1, less than 1.1:1, or less than 1:1.

In one embodiment or in combination with any other embodiment, class or subclass of this first aspect, wherein the MCE has a ratio of hydroxyl substituents to propionyl substituents of at least 0.4:1, at least 0.45:1, at least 0.5:1, at least 0.6:1, at least 0.7:1, at least 0.8:1, at least 0.9:1, at least 1:1, at least 1.1:1, at least 1.2:1, at least 1.3:1, at least 1.4:1, at least 1.5:1, at least 1.6:1, at least 1.7:1, at least 1.8:1, at least 1.9:2, or at least 2:1. Additionally, or in the alternative, the MCE has a ratio of hydroxyl substituents to propionyl substituents of less than 2:1, less than 1.9:1, less than 1.8:1, less than 1.7:1, less than 1.6:1, less than 1.5:1, less than 1.4:1, less than 1.3:1, less than 1.2:1, less than 1.1:1, or less than 1:1; or less than 0.9:1; or less than 0.8:1; or less than 0.7:1; or less than 0.6:1; or less than 0.5:1.

In one embodiment or in combination with any other embodiment, class or subclass of this first aspect, wherein the MCE exhibits at least 40% biodegradability, at least 45% biodegradability, or at least 50% biodegradability, or at least 55% biodegradability, at least 60% biodegradability, or at least 65% biodegradability, or at least 70% biodegradability, or at least 75% biodegradability, or at least 80% biodegradability, or at least 85% biodegradability, at 56 days according to at least one of the OECD 301B, OECD

In one embodiment or in combination with any other embodiment, class or subclass of this first aspect, wherein the MCE exhibits at least 40% biodegradability, at least 45% biodegradability, or at least 50% biodegradability, or at least 55% biodegradability, at least 60% biodegradability, or at least 65% biodegradability, or at least 70% biodegradability, or at least 75% biodegradability, or at least 80% biodegradability, or at least 85% biodegradability, at 60 days according to at least one of the OECD 301B, OECD 301C, or OECD 301F test methods.

In one embodiment or in combination with any other embodiment, class or subclass of this first aspect, the MCE has a weight average molecular weight in the range of from 5,000 to 100,000 Da, or 5,000 to 50,000 Da, or 5,000 to 25,000 Da, or 15,000 to 100,000 Da, or 15,000 to 50,000 Da, or 15,000 to 25,000 Da, or 50,000 to 100,000 Da, or 75,000 to 100,000 Da, or 15,000 to 250,000 Da.

The present application discloses, in a second aspect, a mixed cellulose ester (“MCE”), comprising: (1) a plurality of acetyl substituents; (2) a plurality of propionyl substituents; and (3) a plurality of hydroxyl substituents, wherein: the MCE has an average degree of substitution for the acetyl substituents (“DS_{Ac}”) is from 0.1 to 2.3, or 0.1 to 1.9, or 1.5 to 2.3; the MCE has an average degree of substitution for the propionyl substituents (“DS_{Pr}”) is from 0.1 to 1.5, or 0.1 to 0.6, or 0.1 to 0.3; and the MCE has an average degree of substitution for the hydroxyl substituents (“DS_{OH}”) is from 0.7 to 2.8, or 0.7 to 1.2.

In one embodiment or in combination with any other embodiment, class or subclass of this second aspect, wherein the DS_{Ac} is at least 0.1, at least 0.2, at least 0.3, at least 0.4, at least 0.5, at least 0.6, at least 0.7, at least 0.8, at least 0.9, at least 1.0, at least 1.1, at least 1.2, at least 1.3, at least 1.4, at least 1.5, at least 1.6, at least 1.7, at least 1.8, at least 1.9, or at least 2.0. Additionally, or in the alternative, the DS_{Ac} is less than 2.3, less than 2.2, less than 2.1, less than 2.0, less than 1.9, less than 1.8, less than 1.7, less than 1.6, less than 1.5, less than 1.4, less than 1.3, less than 1.2, less than 1.1, less than 1.0, less than 0.9, less than 0.8, less than 0.7, less than 0.5, less than 0.4, or less than 0.3.

In one embodiment or in combination with any other embodiment, class or subclass of this second aspect, wherein the DS_{Ac} is from 0.6 to 0.7, or 0.6 to 0.8, or 0.6 to 0.9, or 0.6 to 1.0, or 0.6 to 1.1, or 0.6 to 1.2, or 0.6 to 1.3, or 0.6 to 1.4, or 0.6 to 1.5, or 0.6 to 1.6, or 0.6 to 1.7, or 0.6 to 1.8, or 0.6 to 1.9, or 0.6 to 2.0, or 0.6 to 2.1, or 0.7 to 0.9, or 0.7 to 1.0, or 0.7 to 1.1, or 0.7 to 1.2, or 0.7 to 1.3, or 0.7 to 1.4, or 0.7 to 1.5, or 0.7 to 1.6, or 0.7 to 1.7, or 0.7 to 1.8, or 0.7 to 1.9, or 0.7 to 2.0, or 0.7 to 2.1, or 0.8 to 0.9, or 0.8 to 1.0, or 0.8 to 1.1, or 0.8 to 1.2, or 0.8 to 1.3, or 0.8 to 1.4, or 0.8 to 1.5, or 0.8 to 1.6, or 0.8 to 1.7, or 0.8 to 1.8, or 0.8 to 1.9, or 0.8 to 2.0, or 0.8 to 2.1, or 0.9 to 1.0, or 0.9 to 1.1, or 0.9 to 1.2, or 0.9 to 1.3, or 0.9 to 1.4, or 0.9 to 1.5, or 0.9 to 1.6, or 0.9 to 1.7, or 0.9 to 1.8, or 0.9 to 1.9, or 0.9 to 2.0, or 0.9 to 2.1, or 1.0 to 1.1, or 1.0 to 1.2, or 1.0 to 1.3, or 1.0 to 1.4, or 1.0 to 1.5, or 1.0 to 1.6, or 1.0 to 1.7, or 1.0 to 1.8, or 1.0 to 1.9, or 1.0 to 2.0, or 1.0 to 2.1, or 1.1 to 1.2, or 1.1 to 1.3, or 1.1 to 1.4, or 1.1 to 1.5, or 1.1 to 1.6, or 1.1 to 1.7, or 1.1 to 1.8, or 1.1 to 1.9, or 1.1 to 2.0, or 1.1 to 2.1.

In one embodiment or in combination with any other embodiment, class or subclass of this second aspect, wherein the DS_{Pr} is at least 0.1, at least 0.2, at least 0.3, at least 0.4, at least 0.5, at least 0.6, at least 0.7, at least 0.8, at least 0.9, at least 1.0, at least 1.1, at least 1.2, at least 1.3, or at least 1.4. Additionally, or in the alternative, the DS_{Pr} is less than 1.5, less than 1.4, less than 1.3, less than 1.2, less than 1.1, less than 1.0, less than 0.9, less than 0.8, less than 0.7, less than 0.5, less than 0.4, or less than 0.3.

In one embodiment or in combination with any other embodiment, class or subclass of this second aspect, wherein the DS_{Pr} is from 1.05 to 1.35, or 1.05 to 1.3, or 1.05 to 1.25, or 1.05 to 1.2, or 1.05 to 1.15, or 1.05 to 1.1, or 1.1 to 1.4, or 1.1 to 1.35, or 1.1 to 1.3, or 1.1 to 1.25, or 1.1 to 1.2, or 1.1 to 1.15, or 1.15 to 1.4, or 1.15 to 1.35, or 1.15 to 1.3, or 1.15 to 1.25, or 1.15 to 1.2, or 1.2 to 1.4, or 1.2 to 1.35, or 1.2 to 1.3, or 1.2 to 1.25, or 1.25 to 1.4, or 1.25 to 1.35, or 1.25 to 1.3, or 1.3 to 1.4, or 1.3 to 1.35.

In one embodiment or in combination with any other embodiment, class or subclass of this second aspect, wherein the DS_{OH} is at least 0.6, at least 0.7, at least 0.8, at least 0.9, at least 1.0, at least 1.1, at least 1.2, at least 1.3, at least 1.4, at least 1.5, at least 1.6, at least 1.7, at least 1.8, at least 1.9, at least 2.0, at least 2.1, at least 2.2, at least 2.3, at least 2.4, at least 2.5, or at least 2.6. Additionally, or in the alternative, the DS_{OH} is less than 2.8, less than 2.7, less than 2.6, less than 2.5, less than 2.4, less than 2.3, less than 2.2, less than 2.1, less than 2.0, less than 1.9, less than 1.8, less than 1.7, less than 1.6, less than 1.5, less than 1.4, less than 1.3, less than 1.2, less than 1.1, less than 1.0, less than 0.9, or less than 0.8.

In one embodiment or in combination with any other embodiment, class or subclass of this second aspect, wherein the DS_{OH} is from 0.7 to 1.35, or 0.7 to 1.3, or 0.7 to 1.25, or 0.7 to 1.2, or 0.7 to 1.15, or 0.7 to 1.1, or 0.7 to 1.05, or 0.7 to 1.0, or 0.7 to 0.95, or 0.7 to 0.9, or 0.7 to 0.85, or 0.7 to 0.8, or 0.7 to 0.75, or 0.75 to 1.4, or 0.75 to 1.35, or 0.75 to 1.3, or 0.75 to 1.25, or 0.75 to 1.2, or 0.75 to 1.15, or 0.75 to 1.1, or 0.75 to 1.05, or 0.75 to 1.0, or 0.75 to 0.95, or 0.8 to 1.4, or 0.8 to 1.35, or 0.8 to 1.3, or 0.8 to 1.25, or 0.8 to 1.2, or 0.8 to 1.15, or 0.8 to 1.1, or 0.8 to 1.05, or 0.85 to 1.4, or 0.85 to 1.35, or 0.85 to 1.3, or 0.85 to 1.25, or 0.85 to 1.2, or 0.85 to 1.15, or 0.85 to 1.1, or 0.85 to 1.05, or 0.9 to 1.4, or 0.9 to 1.35, or 0.9 to 1.3, or 0.9 to 1.25, or 0.9 to 1.2, or 0.9 to 1.15, or 0.9 to 1.1, or 0.9 to 1.05.

In one embodiment or in combination with any other embodiment, class or subclass of this second aspect, wherein the sum of DS_{Pr} and DS_{Ac} is from 1.65 to 2.3, or 1.65 to 2.2, or 1.65 to 2.1, or 1.65 to 2.0, or 1.65 to 1.9, or 1.65 to 1.8, or 1.7 to 2.3, or 1.7 to 2.2, or 1.7 to 2.1, or 1.7 to 2.0, or 1.7 to 1.9, or 1.7 to 1.8, or 1.75 to 2.3, or 1.75 to 2.2, or 1.75 to 2.1, or 1.75 to 2.0, or 1.75 to 1.9, or 1.8 to 2.3, or 1.8 to 2.2, or 1.8 to 2.1, or 1.8 to 2.0, or 1.8 to 1.9, or 1.9 to 2.3, or 1.9 to 2.2, or 1.9 to 2.1, or 1.9 to 2.0, or 2.0 to 2.3, or 2.0 to 2.2, or 2.0 to 2.1.

In one embodiment or in combination with any other embodiment, class or subclass of this second aspect, wherein the DS_{OH} is from 0.6 to 0.7, or 0.7 to 1.35, or 0.7 to 1.3, or 0.7 to 1.25, or 0.7 to 1.2, or 0.7 to 1.15, or 0.7 to 1.1, or 0.7 to 1.05, or 0.7 to 1.0, or 0.7 to 0.95, or 0.7 to 0.9, or 0.7 to 0.85, or 0.7 to 0.8, or 0.7 to 0.75, or 0.75 to 1.4, or 0.75 to 1.35, or 0.75 to 1.3, or 0.75 to 1.25, or 0.75 to 1.2, or 0.75 to 1.15, or 0.75 to 1.1, or 0.75 to 1.05, or 0.75 to 1.0, or 0.75 to 0.95, or 0.8 to 1.4, or 0.8 to 1.35, or 0.8 to 1.3, or 0.8 to 1.25, or 0.8 to 1.2, or 0.8 to 1.15, or 0.8 to 1.1, or 0.8 to 1.05, or 0.85 to 1.4, or 0.85 to 1.35, or 0.85 to 1.3, or 0.85 to 1.25, or 0.85 to 1.2, or 0.85 to 1.15, or 0.85 to 1.1, or 0.85 to 1.05, or 0.9 to 1.4, or 0.9 to 1.35, or 0.9 to 1.3, or 0.9 to 1.25, or 0.9 to 1.2, or 0.9 to 1.15, or 0.9 to 1.1, or 0.9 to 1.05.

In one embodiment or in combination with any other embodiment, class or subclass of this second aspect, wherein the mixed cellulose ester has a ratio of hydroxyl substituents to acetyl substituents of at least 0.4:1, at least 0.5:1, at least 0.6:1, at least 0.7:1, at least 0.8:1, at least 0.9:1, at least 1:1, at least 1.1:1, at least 1.2:1, at least 1.3:1, at least 1.4:1, at least 1.5:1, at least 1.6:1, at least 1.7:1, at least 1.8:1, at least 1.9:2, or at least 2:1. Additionally, in the alternative, the mixed cellulose ester has a ratio of hydroxyl substituents to acetyl substituents of less than 2:1, less than 1.9:1, less than 1.8:1, less than 1.7:1, less than 1.6:1, less than 1.5:1, less than 1.4:1, less than 1.3:1, less than 1.2:1, less than 1.1:1, or less than 1:1.

In one embodiment or in combination with any other embodiment, class or subclass of this second aspect, wherein the MCE has a ratio of hydroxyl substituents to propionyl substituents of at least 0.4:1, at least 0.5:1, at least 0.6:1, at least 0.7:1, at least 0.8:1, at least 0.9:1, at least 1:1, at least 1.1:1, at least 1.2:1, at least 1.3:1, at least 1.4:1, at least 1.5:1, at least 1.6:1, at least 1.7:1, at least 1.8:1, at least 1.9:2, or at least 2:1, or at least 3:1, or at least 4:1. Additionally, or in the alternative, the MCE has a ratio of hydroxyl substituents to propionyl substituents of less than 6:1, less than 5:1, less than 4:1, less than 3:1, less than 2:1, less than 1.9:1, less than 1.8:1, less than 1.7:1, less than 1.6:1, less than 1.5:1, less than 1.4:1, less than 1.3:1, less than 1.2:1, less than 1.1:1, or less than 1:1.

In one embodiment or in combination with any other embodiment, class or subclass of this second aspect, wherein the MCE exhibits at least 40% biodegradability, at least 45% biodegradability, or at least 50% biodegradability, or at least 55% biodegradability, at least 60% biodegradability, or at least 65% biodegradability, or at least 70% biodegradability, or at least 75% biodegradability, or at least 80% biodegradability, or at least 85% biodegradability, at 56 days according to at least one of the OECD 301B, OECD 301C, or OECD 301F test methods.

In one embodiment or in combination with any other embodiment, class or subclass of this second aspect, wherein the MCE exhibits at least 40% biodegradability, at least 45% biodegradability, or at least 50% biodegradability, or at least 55% biodegradability, at least 60% biodegradability, or at least 65% biodegradability, or at least 70% biodegradability, or at least 75% biodegradability, or at least 80% biodegradability, or at least 85% biodegradability, at 60 days according to at least one of the OECD 301B, OECD 301C, or OECD 301F test methods.

In one embodiment or in combination with any other embodiment, class or subclass of this second aspect, the MCE has a weight average molecular weight in the range of from 5,000 to 100,000 Da, or 5,000 to 50,000 Da, or 5,000 to 25,000 Da, or 15,000 to 100,000 Da, or 15,000 to 50,000 Da, or 15,000 to 25,000 Da, or 50,000 to 100,000 Da, or 75,000 to 100,000 Da, or 15,000 to 250,000 Da.

The present application, in a third aspect, also discloses a mixed cellulose ester (“MCE”), comprising: (1) a plurality of acetyl substituents; (2) a plurality of butyryl substituents; and (3) a plurality of hydroxyl substituents, wherein: the MCE has an average degree of substitution for the acetyl substituents (“DS_{Ac}”) is from 0.1 to 2.4, or 1.0 to 2.4, or 1.5 to 2.4; the MCE has an average degree of substitution for the butyryl substituents (“DS_{Bu}”) is from 0.1 to 1.5, or 0.1 to 0.6, or 0.1 to 0.3; the MCE has an average degree of substitution for the hydroxyl substituents (“DS_{OH}”) is from 0.6 to 2.8, or 0.6 to 1.5, or 0.6 to 1.2.

In one embodiment or in combination with any other embodiment, class or subclass of this first aspect, wherein the DS_{Ac} is at least 0.1, at least 0.2, at least 0.3, at least 0.4, at least 0.5, at least 0.6, at least 0.7, at least 0.8, at least 0.9, at least 1.0, at least 1.1, at least 1.2, at least 1.3, at least 1.4, at least 1.5, at least 1.6, at least 1.7, at least 1.8, at least 1.9, or at least 2.0. Additionally, or in the alternative, the DS_{Ac} is less than 2.3, less than 2.2, less than 2.1, less than 2.0, less than 1.9, less than 1.8, less than 1.7, less than 1.6, less than 1.5, less than 1.4, less than 1.3, less than 1.2, less than 1.1, less than 1.0, less than 0.9, less than 0.8, less than 0.7, less than 0.5, less than 0.4, or less than 0.3.

In one embodiment or in combination with any other embodiment, class or subclass of this third aspect, wherein the DS_{Ac} is from 0.9 to 2.4, 0.9 to 2.3, or 0.9 to 2.2, or 0.9 to 2.1, or 0.9 to 2.0, or 0.9 to 1.9, or 0.9 to 1.8, or 0.9 to 1.7, or 0.9 to 1.6, or 0.9 to 1.4, 0.9 to 1.3, or 0.9 to 1.2, or 0.9 to 1.1, or 0.9 to 1.0, or 0.92 to 2.4, 0.92 to 2.3, or 0.92 to 2.2, or 0.92 to 2.1, or 0.92 to 2.0, or 0.92 to 1.9, or 0.92 to 1.8, or 0.92 to 1.7, or 0.92 to 1.6, or 0.92 to 1.4, 0.92 to 1.3, or 0.92 to 1.2, or 0.92 to 1.1, or 0.92 to 1.0, or 0.94 to 2.4, 0.94 to 2.3, or 0.94 to 2.2, or 0.94 to 2.1, or 0.94 to 2.0, or 0.94 to 1.9, or 0.94 to 1.8, or 0.94 to 1.7, or 0.94 to 1.6, or 0.94 to 1.4, 0.94 to 1.3, or 0.94 to 1.2, or 0.94 to 1.1, or 0.94 to 1.0, or 0.96 to 2.4, 0.96 to 2.3, or 0.96 to 2.2, or 0.96 to 2.1, or 0.96 to 2.0, or 0.96 to 1.9, or 0.96 to 1.8, or 0.96 to 1.7, or 0.96 to 1.6, or 0.96 to 1.4, 0.96 to 1.3, or 0.96 to 1.2, or 0.96 to 1.1, or 0.96 to 1.0, or 0.98 to 2.4, 0.98 to 2.3, or 0.98 to 2.2, or 0.98 to 2.1, or 0.98 to 2.0, or 0.98 to 1.9, or 0.98 to 1.8, or 0.98 to 1.7, or 0.98 to 1.6, 0.98 to 1.4, 0.98 to 1.3, or 0.98 to 1.2, or 0.98 to 1.1, or 0.98 to 1.0, or 1.0 to 2.4, 1.0 to 2.3, or 1.0 to 2.2, or 1.0 to 2.1, or 1.0 to 2.0, or 1.0 to 1.9, or 1.0 to 1.8, or 1.0 to 1.7, or 1.0 to 1.6, or 1.0 to 1.4, or 1.0 to 1.3, 1.0 to 1.2, or 1.0 to 1.1, or 1.1 to 2.4, or 1.1 to 2.3, or 1.1 to 2.2, or 1.1 to 2.1, or 1.1 to 2.0, or 1.1 to 1.9, or 1.1 to 1.8, or 1.1 to 1.7, or 1.1 to 1.6, 1.1 to 1.4, or 1.1 to 1.3, or 1.1 to 1.2, or 1.2 to 2.4, or 1.2 to 2.3, or 1.2 to 2.2, or 1.2 to 2.1, or 1.2 to 2.0, or 1.2 to 1.9, or 1.2 to 1.8, or 1.2 to 1.7, or 1.2 to 1.6, or 1.2 to 1.4, or 1.2 to 1.3, or 1.3 to 2.4, or 1.3 to 2.3, or 1.3 to 2.2, or 1.3 to 2.1, or 1.3 to 2.0, or 1.3 to 1.9, or 1.3 to 1.8, or 1.3 to 1.7, or 1.3 to 1.6, or 1.3 to 1.4, or 1.4 to 2.4, or 1.4 to 2.3, or 1.4 to 2.2, or 1.4 to 2.1, or 1.4 to 2.0, or 1.4 to 1.9, or 1.4 to 1.8, or 1.4 to 1.7, or 1.4 to 1.6, or 1.5 to 2.4, or 1.5 to 2.3, or 1.5 to 2.2, or 1.5 to 2.1, or 1.5 to 2.0, or 1.5 to 1.9, or 1.5 to 1.8, or 1.5 to 1.7, or 1.5 to 1.6, or 1.6 to 2.4, or 1.6 to 2.3, or 1.6 to 2.2, or 1.6 to 2.1, or 1.6 to 2.0, or 1.6 to 1.9, or 1.6 to 1.8, or 1.6 to 1.7, or 1.7 to 2.4, or 1.7 to 2.3, or 1.7 to 2.2, or 1.7 to 2.1, or 1.7 to 2.0, or 1.7 to 1.9, or 1.7 to 1.8, or 1.8 to 2.3, or 1.8 to 2.1, or 1.8 to 2.0, or 1.8 to 1.9, or 1.9 to 2.3, or 1.9 to 2.2, or 1.9 to 2.1, or 1.9 to 2.0, or 2.0 to 2.4, or 2.0 to 2.3, or 2.0 to 2.2, or 2.0 to 2.1, or 2.1 to 2.4, or 2.1 to 2.3, or 2.1 to 2.2, or 2.2 to 2.3.

In one embodiment or in combination with any other embodiment, class or subclass of this third aspect, wherein the DS_{Bu} is at least 0.05, at least 0.1, at least 0.15, at least 0.2, at least 0.3, at least 0.4, at least 0.5, at least 0.6, at least 0.7, at least 0.8, at least 0.9, at least 1.0, at least 1.1, at least 1.2, at least 1.3, or at least 1.4. Additionally, or in the alternative, the DS_{Bu} is less than 1.5, less than 1.4, less than 1.3, less than 1.2, less than 1.1, less than 1.0, less than 0.9, less than 0.8, less than 0.7, less than 0.5, less than 0.4, less than 0.35, less than 0.3, or less than 0.25.

In one embodiment or in combination with any other embodiment, class or subclass of this third aspect, wherein the DS_{Bu} is from 0.1 to 1.35, or 0.1 to 1.3, or 0.1 to 1.25, or 0.1 to 1.2, or 0.1 to 1.15, or 0.1 to 1.1, or 0.1 to 1.0, or 0.1 to 0.8, or 0.1 to 0.6, or 0.1 to 0.5, or 0.1 to 0.4, or 0.1 to 0.3, or 0.1 to 0.25, or 0.15 to 1.35, or 0.15 to 1.3, or 0.15 to 1.25, or 0.15 to 1.2, or 0.15 to 1.15, or 0.15 to 1.1, or 0.15 to 1.0, or 0.15 to 0.8, or 0.15 to 0.6, or 0.15 to 0.5, or 0.15 to 0.4, or 0.15 to 0.3, or 0.15 to 0.25, or 0.2 to 1.35, or 0.2 to 1.3, or 0.2 to 1.25, or 0.2 to 1.2, or 0.2 to 1.15, or 0.2 to 1.1, or 0.2 to 1.0, or 0.2 to 0.8, or 0.2 to 0.6, or 0.2 to 0.4, or 0.3 to 1.35, or 0.3 to 1.3, or 0.3 to 1.25, or 0.3 to 1.2, or 0.3 to 1.15, or 0.3 to 1.1, or 0.3 to 1.0, or 0.3 to 0.8, or 0.3 to 0.6, or 0.3 to 0.5, or 0.4 to 1.35, or 0.4 to 1.3, or 0.4 to 1.25, or 0.4 to 1.2, or 0.4 to 1.15, or 0.4 to 1.1, or 0.4 to 1.0, or 0.4 to 0.8, or 0.4 to 0.6, or 0.5 to 1.35, or 0.5 to 1.3, or 0.5 to 1.25, or 0.5 to 1.2, or 0.5 to 1.15, or 0.5 to 1.1, or 0.5 to 1.0, or 0.5 to 0.8, or 0.5 to 0.7, or 0.6 to 1.35, or 0.6 to 1.3, or 0.6 to 1.25, or 0.6 to 1.2, or 0.6 to 1.15, or 0.6 to 1.1, or 0.6 to 1.0, or 0.6 to 0.8, or 0.7 to 1.35, or 0.7 to 1.3, or 0.7 to 1.25, or 0.7 to 1.2, or 0.7 to 1.15, or 0.7 to 1.1, or 0.7 to 1.0, or 0.8 to 1.35, or 0.8 to 1.3, or 0.8 to 1.25, or 0.8 to 1.2, or 0.8 to 1.15, or 0.8 to 1.1, or 0.8 to 1.0, or 0.9 to 1.35, or 0.9 to 1.3, or 0.9 to 1.25, or 0.9 to 1.2, or 0.9 to 1.15, or 0.9 to 1.1, or 1.0 to 1.35, or 1.0 to 1.3, or 1.0 to 1.25, or 1.0 to 1.2, or 1.0 to 1.15, or 1.0 to 1.1, or 1.05 to 1.35, or 1.05 to 1.3, or 1.05 to 1.25, or 1.05 to 1.2, or 1.05 to 1.15, or 1.05 to 1.1, or 1.1 to 1.4, or 1.1 to 1.35, or 1.1 to 1.3, or 1.1 to 1.25, or 1.1 to 1.2, or 1.1 to 1.15, or 1.15 to 1.4, or 1.15 to 1.35, or 1.15 to 1.3, or 1.15 to 1.25, or 1.15 to 1.2, or 1.2 to 1.4, or 1.2 to 1.35, or 1.2 to 1.3, or 1.2 to 1.25, or 1.25 to 1.4, or 1.25 to 1.35, or 1.25 to 1.3, or 1.3 to 1.4, or 1.3 to 1.35.

In one embodiment or in combination with any other embodiment, class or subclass of this third aspect, wherein the DS_{OH} is at least 0.5, at least 0.55, at least 0.6, at least 0.7, at least 0.8, at least 0.9, at least 1.0, at least 1.1, at least 1.2, at least 1.3, at least 1.4, at least 1.5, at least 1.6, at least 1.7, at least 1.8, at least 1.9, at least 2.0, at least 2.1, at least 2.2, at least 2.3, at least 2.4, at least 2.5, or at least 2.6. Additionally, or in the alternative, the DS_{OH} is less than 2.8, less than 2.7, less than 2.6, less than 2.5, less than 2.4, less than 2.3, less than 2.2, less than 2.1, less than 2.0, less than 1.9, less than 1.8, less than 1.7, less than 1.6, less than 1.5, less than 1.4, less than 1.3, less than 1.2, less than 1.1, less than 1.0, less than 0.9, less than 0.85, or less than 0.8.

In one embodiment or in combination with any other embodiment, class or subclass of this third aspect, wherein the DS_{OH} is from 0.5 to 1.3, 0.5 to 1.2, 0.5 to 1.1, 0.5 to 1.0, or 0.5 to 0.95, or 0.5 to 0.9, or 0.5 to 0.85, or 0.5 to 0.8, or 0.5 to 0.75, or 0.5 to 0.7, or 0.5 to 0.65, or 0.5 to 0.6, or 0.5 to 0.55, or 0.55 to 1.0, or 0.55 to 0.95, or 0.55 to 0.9, or 0.55 to 0.85, or 0.55 to 0.8, or 0.55 to 0.75, or 0.55 to 0.7, or 0.55 or 0.65, or 0.55 to 0.6, or 0.6 to 0.65, or 0.6 to 0.7, or 0.6 to 0.75, or 0.6 to 0.8, or 0.6 to 0.85, or 0.6 to 0.9, or 0.6 to 0.95, or 0.6 to 1.0, or 0.65 to 0.7, or 0.65 to 0.75, or 0.65 to 0.8, or 0.65 to 0.85, or 0.65 to 0.9, or 0.65 to 0.95, or 0.65 to 1.0.

In one embodiment or in combination with any other embodiment, class or subclass of this third aspect, wherein the sum of DS_{Bu} and DS_{Ac} is from 1.65 to 2.3, or 1.65 to 2.2, or 1.65 to 2.1, or 1.65 to 2.0, or 1.65 to 1.9, or 1.65 to 1.8, or 1.7 to 2.3, or 1.7 to 2.2, or 1.7 to 2.1, or 1.7 to 2.0, or 1.7 to 1.9, or 1.7 to 1.8, or 1.75 to 2.3, or 1.75 to 2.2, or 1.75 to 2.1, or 1.75 to 2.0, or 1.75 to 1.9, or 1.8 to 2.3, or 1.8 to 2.2, or 1.8 to 2.1, or 1.8 to 2.0, or 1.8 to 1.9, or 1.9 to 2.3, or 1.9 to 2.2, or 1.9 to 2.1, or 1.9 to 2.0, 2.0 to 2.4, or 2.0 to 2.3, or 2.0 to 2.2, or 2.0 to 2.1.

In one embodiment or in combination with any other embodiment, class or subclass of this third aspect, wherein the mixed cellulose ester has a ratio of hydroxyl substituents to acetyl substituents of at least 0.4:1, at least 0.45:1, at least 0.5:1, at least 0.6:1, at least 0.7:1, at least 0.8:1, at least 0.9:1, at least 1:1, at least 1.1:1, at least 1.2:1, at least 1.3:1, at least 1.4:1, at least 1.5:1, at least 1.6:1, at least 1.7:1, at least 1.8:1, at least 1.9:2, or at least 2:1. Additionally, in the alternative, the mixed cellulose ester has a ratio of hydroxyl substituents to acetyl substituents of less than 2:1, less than 1.9:1, less than 1.8:1, less than 1.7:1, less than 1.6:1, less than 1.5:1, less than 1.4:1, less than 1.3:1, less than 1.2:1, less than 1.1:1, less than 1:1, less than 0.9:1, less than 0.8:1, less than 0.7:1, less than 0.6:1, or less than 0.5:1.

In one embodiment or in combination with any other embodiment, class or subclass of this third aspect, wherein the mixed cellulose ester has a ratio of hydroxyl substituents to butyryl substituents of at least 0.4:1, at least 0.5:1, at least 0.6:1, at least 0.7:1, at least 0.8:1, at least 0.9:1, at least 1:1, at least 1.1:1, at least 1.2:1, at least 1.3:1, at least 1.4:1, at least 1.5:1, at least 1.6:1, at least 1.7:1, at least 1.8:1, at least 1.9:2, at least 2:1, at least 3:1, or at least 4:1. Additionally, or in the alternative, the mixed cellulose ester has a ratio of hydroxyl substituents to butyryl substituents of less than 6:1, less than 5:1, less than 4:1, less than 3:1, less than 2:1, less than 1.9:1, less than 1.8:1, less than 1.7:1, less than 1.6:1, less than 1.5:1, less than 1.4:1, less than 1.3:1, less than 1.2:1, less than 1.1:1, or less than 1:1.

In one embodiment or in combination with any other embodiment, class or subclass of this third aspect, wherein the MCE exhibits at least 40% biodegradability, at least 45% biodegradability, or at least 50% biodegradability, or at least 55% biodegradability, at least 60% biodegradability, or at least 65% biodegradability, or at least 70% biodegradability, or at least 75% biodegradability, or at least 80% biodegradability, or at least 85% biodegradability, at 56 days according to at least one of the OECD 301B, OECD

In one embodiment or in combination with any other embodiment, class or subclass of this third aspect, wherein the MCE exhibits at least 40% biodegradability, at least 45% biodegradability, or at least 50% biodegradability, or at least 55% biodegradability, at least 60% biodegradability, or at least 65% biodegradability, or at least 70% biodegradability, or at least 75% biodegradability, or at least 80% biodegradability, or at least 85% biodegradability, at 60 days according to at least one of the OECD 301B, OECD 301C, or OECD 301F test methods.

In one embodiment or in combination with any other embodiment, class or subclass of this third aspect, the MCE has a weight average molecular weight in the range of from 5,000 to 100,000 Da, or 5,000 to 50,000 Da, or 5,000 to 25,000 Da, or 15,000 to 100,000 Da, or 15,000 to 50,000 Da, or 15,000 to 25,000 Da, or 50,000 to 100,000 Da, or 75,000 to 100,000 Da, or 15,000 to 250,000 Da.

In one embodiment or in combination with any embodiment mentioned herein, the mixed cellulose ester of the first aspect, the second aspect, and/or the third aspect, including any class or subclass of these aspects, have a butyric acid content of less than 500, 400, 300, 200, 100, 50, 20, 10, 7.5, 5, 2.5, or 1 ppmw.

In one embodiment or in combination with any embodiment mentioned herein, the mixed cellulose ester of the first aspect, the second aspect, and/or the third aspect, including any class or subclass of these aspects, have an acetic acid content of less than 500, 400, 300, 200, 100, 50, 20, 10, 7.5, 5, 2.5, or 1 ppmw.

In one embodiment or in combination with any embodiment mentioned herein, the mixed cellulose ester of the first aspect, the second aspect, and/or the third aspect, including any class or subclass of these aspects, have a propionic acid content of less than 500, 400, 300, 200, 100, 50, 20, 10, 7.5, 5, 2.5, or 1 ppmw.

In one embodiment or in combination with any embodiment mentioned herein, the mixed cellulose ester of the first aspect, the second aspect, and/or the third aspect, including any class or subclass of these aspects, have a sulfuric acid content of less than 500, 400, 300, 200, 100, 50, 20, 10, 7.5, 5, 2.5, or 1 ppmw.

In one embodiment or in combination with any embodiment mentioned herein, the CE 100 can be the mixed cellulose ester of the first aspect, the second aspect, and/or the third aspect, including any class or subclass of these aspects.

### Solvent System

The solvent system, such as solvent 102, is generally capable of solubilizing CE to produce the dispersed/solid phase of an emulsion/dispersion as described herein. In one embodiment or in combination with any embodiment mentioned herein, solvent 102 may consist of a single solvent component, or may be a solvent system including a plurality of solvent components. The plurality of solvent components may include at least two solvent components, at least three solvent components, or three total solvent components.

In one embodiment or in combination with any embodiment mentioned herein, solvent 102 includes at least one, at least two, or all three of a C1-C4 alkyl acetate, a C1-C4 alcohol, and water. The C1-C4 alkyl acetate may include one or more of methyl acetate, ethyl acetate, n-propyl acetate, isopropyl acetate, n-butyl acetate, isobutyl acetate, and sec-butyl acetate. The C1-C4 alcohol may include one or more of methanol, ethanol, propanols (e.g., isopropanol, n-propanol, and isopropyl alcohol), and butanols (e.g., n-butanol, isobutanol, sec-butanol, and tert-butanol).

In one embodiment or in combination with any embodiment mentioned herein, when solvent 102 includes more than one solvent component, the C1-C4 alkyl acetate may be present in one or more of the following amounts: (1) at least 10, 25, 50, 60, or 70 weight percent; (2) not more than 99, 95, 90, 85, or 80 weight percent; and (3) in the range of 10-99, 25-95, 50-90, 70-85 weight percent.

In one embodiment or in combination with any embodiment mentioned herein, when solvent 102 includes more than one solvent component, the C1-C4 alcohol is present in one or more of the following amounts: (1) at least 1, 2, 4, 6, 8, or 10 weight percent; (2) not more than 80, 60, 40, 30, 20, or 15 weight percent; and (3) in the range or 1-80, 2-60, 4-40, 6-30, 8-20, or 10-15 weight percent.

In one embodiment or in combination with any embodiment mentioned herein, when solvent 102 includes more than one solvent component, the water is present in one or more of the following amounts: (1) at least 1, 2, 4, 6, or 8 weight percent; (2) not more than 50, 25, 20, or 15 weight percent; and (3) in the range of 1-50, 2-25, 4-30, 6-20, or 8-15 weight percent.

In one embodiment or in combination with any embodiment mentioned herein, solvent 102 includes at least one, at least two, or all three of ethyl acetate, n-propanol, and water. In such an embodiment, ethyl acetate is present in an amount in the range of 50-90, 55-90, 60-90, 65-90, 65-85, 70-85, or 75-85 weight percent, n-propanol is present in an amount in the range of 4-40, 5-35, 6-30, 7-25, 8-20, 10-20, or 10-15 weight percent, and water is present in an amount in the range of 4-30, 5-25, 6-20, 7-15, 8-12, or 9-12 weight percent.

Surprisingly, it has been found that water contributes and/or enables certain mixed cellulose esters to be solubilized. Particularly, it has been found that water contributes and/or enables mixed cellulose esters having high degrees of biodegradability, such as those associated with DS_{OH} values greater than a determined threshold, to be solubilized. These findings are disclosed in the EXPERIMENTS section below.

### Hydrocolloid

Hydrocolloids as described herein, such as hydrocolloid 106, are used as a colloidal protector and/or viscosity builder. In one embodiment or in combination with any embodiment mentioned herein, hydrocolloid 106 is a lyophilic colloid. For example, hydrocolloid 106 may include at least one of a gelatin, a natural gum, a protein, or a cellulose derivative. The cellulose derivative may include one or both of methyl cellulose, and carboxy methyl cellulose.

The hydrocolloid, such as carboxy methyl cellulose, may be selected based on a desired viscosity of the resulting aqueous mixture. In one embodiment or in combination with any embodiment mentioned herein, a “low” viscosity hydrocolloid has a viscosity in a range between 10-50 cps, a “medium” viscosity hydrocolloid has a viscosity in a range between 400-800 cps, and a “high” viscosity hydrocolloid has a viscosity in a range between 1500-3000 cps.

### Surfactant System

In one embodiment or in combination with any embodiment mentioned herein, surfactant 108 includes two or more individual emulsifiers. The individual emulsifiers may be differentiated from each other based on their Hydrophilic-Lipophilic Balance (HLB) numbers. For example, when surfactant 108 includes two individual emulsifiers, the emulsifiers may include a lower-HLB emulsifier, and a higher-HLB emulsifier.

In one embodiment or in combination with any embodiment mentioned herein, the HLB number of the higher-HLB emulsifier is at least 6, 8, 10, 12, 14, 16, or 18, and the HLB number of the lower-HLB emulsifier not more than 12, 10, 8, 6, or 4.

In one embodiment or in combination with any embodiment mentioned herein, the HLB number of the higher-HLB emulsifier is greater than the HLB number of the lower-HLB emulsifier by at least one of the following: (1) at least 2, 4, 8, 10, 12, or 14; (2) not more than 25, 20, or 15; or (3) in the range of 2-25, 8-20, or 12-15.

In one embodiment or in combination with any embodiment mentioned herein, the lower-HLB emulsifier is a glycerol ester of stearic acid. In one embodiment or in combination with any embodiment mentioned herein, the higher-HLB emulsifier is a secondary alcohol ethoxylate.

In one embodiment or in combination with any embodiment mentioned herein, surfactant 108 further includes a third emulsifier. The third emulsifier has an HLB number greater than the HLB number of the lower-HLB emulsifier. In one embodiment or in combination with any embodiment mentioned herein, the third emulsifier is a polyethylene glycol ester of stearic acid.

When the surfactant 108 is formed from all three emulsifiers, the lower-HLB emulsifier and the third emulsifier may be present in a ratio that is at least 0.25:1, 0.5:1, 0.75:1, 1:1, 1.25:1, 1.5:1, 1.75:1, or 2:1 and/or not more than 5:1, 4:1, 3:1, 2:1, 1.75:1, 1.5:1, or 1.25:1. The lower-HLB emulsifier and the third emulsifier may define a combined emulsifier. In addition, the higher-HLB emulsifier and the combined emulsifier may be present in surfactant 108 in a ratio that is at least 0.25:1, 0.5:1, 0.75:1, 1:1, 1.25:1, 1.5:1, 1.75:1, or 2:1 and/or not more than 5:1, 4:1, 3:1, 2:1, 1.75:1, 1.5:1, or 1.25:1.

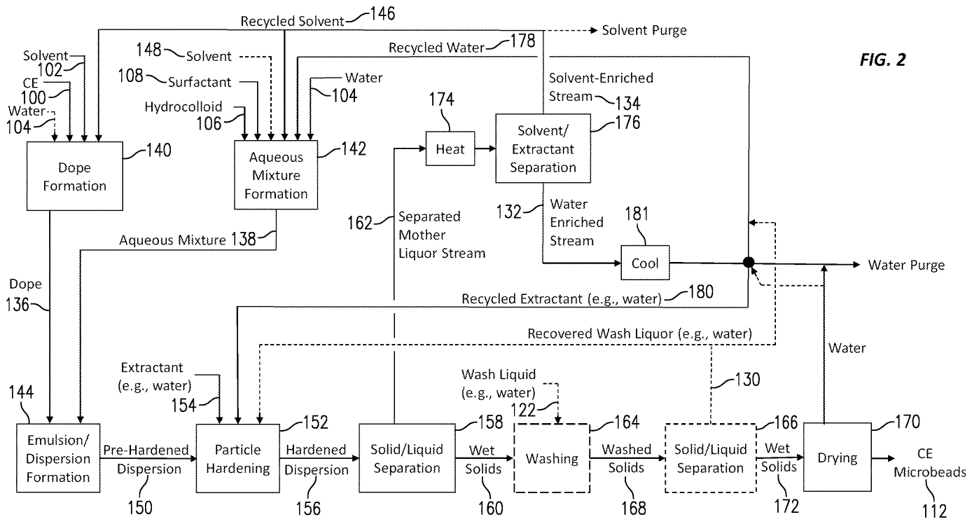

Referring now to FIG. 2, the illustrated process for making CE microparticles 112 includes separate formation of a CE dope 136 and an aqueous mixture 138. CE dope 136 and aqueous mixture 138 are then combined to form CE microparticles 112. For example, CE dope 136 may be formed at unit 140, and aqueous mixture 138 may be formed at unit 142. CE dope 136 and aqueous mixture 138 may then be combined at unit 144 to form an emulsion and/or dispersion, as will be described in more detail below.

In one embodiment or in combination with any embodiment mentioned herein, CE dope 136 is formed from CE 100, solvent 102, water 104, and, in some embodiments, recycled solvent 146 derived from solvent-enriched stream 134.

In one embodiment or in combination with any embodiment mentioned herein, CE 100 is present in CE dope in one or more of: (1) at least 1, 2, 4, 6, 8, or 10 weight percent; (2) not more than 80, 60, 40, 30, 20, or 15 weight percent; and (3) in the range 1-80, 2-60, 4-40, 6-30, 8-20, or 10-15 weight percent.

In one embodiment or in combination with any embodiment mentioned herein, when solvent 102 includes a C1-C4 alkyl acetate, the C1-C4 alkyl acetate is present in CE dope 136 in one or more of the following amounts: (1) at least 10, 25, 50, 60, or 65 weight percent; (2) not more than 95, 90, 85, 80 or 75 weight percent; and (3) in the range 10-95, 25-90, 50-85, 65-75 weight percent.

In one embodiment or in combination with any embodiment mentioned herein, when solvent 102 includes a C1-C4 alcohol, the C1-C4 alcohol is present in CE dope 136 in one or more of the following amounts: (1) at least 1, 2, 4, 6, or 8 weight percent; (2) not more than 50, 25, 20, or 15 weight percent; and (3) in the range of 1-50, 2-25, 4-30, 6-20, or 8-15 weight percent.

In one embodiment or in combination with any other embodiment, the ratio of solvent to CE used in the dissolving to form CE dope 136 is at least 1:1, 2:1, 3:1, 4:1, or 5:1 and/or not more than 100:1, 50:1, 25:1, or 10:1.

In one embodiment or in combination with any embodiment mentioned herein, when solvent 102 includes water, the water is present in CE dope 136 in one or more of the following amounts: (1) at least 0.5, 1, 2, 4, or 6 weight percent; (2) not more than 40, 25, 15, or 10 weight percent; and (3) in the range of 0.5-40, 1-25, 2-15, 4-20, or 6-10 weight percent.

In one particular embodiment, CE dope 136 is formed from CE 100, solvent 102 including ethyl acetate and n-propanol, and water 104. In such an embodiment, CE is present in CE dope 136 in an amount in the range of 6-30, 7-25, 8-20, 9-20, 10-15, or 12-15 weight percent, ethyl acetate is present in CE dope 136 in an amount in the range of 50-85, 55-85, 60-85, 65-85, 70-85, or 75-85 weight percent, n-propanol is present in CE dope 136 in an amount in the range of 4-30, 6-25, 8-20, 10-20, or 12-15 weight percent, and water is present in CE dope 136 in an amount in the range of 4-20, 5-18, 6-16, 7-14, or 8-12 weight percent.

In one embodiment or in combination with any embodiment mentioned herein, CE 100, solvent 102, and water 104 are combined at unit 140 until substantially homogeneous to produce CE dope 136. In one embodiment or in combination with any embodiment mentioned herein, these components are mixed at room temperature (e.g., at least 15, 20, 25, or 30° C. and/or not more than 45, 40, 35, 30, 25, or 20° C.) for a duration of at least 1, 2, 5, 10, 15, 20, 25, 30, 45, 60, 120, or 240 minutes and/or for as long as is needed to produce the substantially homogeneous mixture.

In one embodiment or in combination with any embodiment mentioned herein, aqueous mixture 138 is formed from water 104, hydrocolloid 106, surfactant 108, and, in some embodiments, recycled solvent 146 derived from solvent-enriched stream 134. Optionally, additional solvent 148 may be added to units 140 and/or 142 as needed to maintain concentration(s) of solvent components at suitable levels.

In one embodiment or in combination with any embodiment mentioned herein, the ratio of the recycled solvent portion to the fresh solvent portion used in said units 140 and/or 142 is at least 2.5:1, 10:1, 25:1, 50:1, 75:1, 90:1, 95:1, or 99:1 by weight and/or not more 1000:1, 500:1, 200:1 or 100:1 by weight.

In one embodiment or in combination with any embodiment mentioned herein, the compositional make-up of the recycled solvent varies from the composition make-up of said fresh solvent by not more than 10, 5, 2, or 1 weight percent total.

In one embodiment or in combination with any embodiment mentioned herein, the fresh solvent and the recycled solvent portion have substantially the same composition.

In one embodiment or in combination with any embodiment mentioned herein, water is present in aqueous mixture 138 in one or more of the following amounts: (1) at least 40, 60, 70, 80, or 85 weight percent; (2) not more than 99, 97, 95, 94, or 92 weight percent; and (3) in the range of 40-99, 70-95, or 85-92 weight percent.

In one embodiment or in combination with any embodiment mentioned herein, the hydrocolloid is present in aqueous mixture 138 in one or more of the following amounts: (1) at least 0.001, 0.005, 0.01, 0.05, or 0.1 weight percent; (2) not more than 15, 10, 5, 2, or 1 weight percent; and (3) in the range of 0.001-15, 0.01-5, or 0.1-2 weight percent.

In one embodiment or in combination with any embodiment mentioned herein, the surfactant is present in aqueous mixture 138 in one or more of the following amounts: (1) at least 0.005, 0.01, 0.05, 0.1, or 0.5 weight percent; (2) not more than 15, 10, 5, 2, or 1.5 weight percent; and (3) in the range of 0.005-15, 0.05-5, or 0.5-1.5 weight percent.

As described above, the surfactant described herein may include a higher-HLB emulsifier and a lower-HLB emulsifier. In such embodiments, the higher-HLB emulsifier and the lower-HLB emulsifier are present in aqueous mixture 138 in a high-to-low HLB emulsifier ratio in the range of at least 0.25:1, 0.5:1, 1:1, 1.5:1, or 1.75:1 and/or not more than 10:1, 5:1, 3:1, or 2.5:1 and/or in the range of 0.25:1-10:1, 0.5:1-5:1, or 1:1-3:1.

Referring again to FIG. 2, in some embodiments, additional solvent 148 and/or recycled solvent 146 is received at unit 142. In such embodiments, the solvent used to form CE dope 136 and aqueous mixture 138 is a common C1-C4 alkyl acetate. Using the at least one common component in the solvent systems received at units 140 and 142 facilitates simplification of separation, recovery, and re-use of the solvent as described herein.

In one embodiment or in combination with any embodiment mentioned herein, when the aqueous mixture 138 include a C1-C4 alkyl acetate, the C1-C4 alkyl acetate is present in aqueous mixture 138 in one or more of the following amounts: (1) at least 1, 2, 4, 6, or 8 weight percent; (2) not more than 50, 40, 30, 20, or 15 weight percent; and (3) in the range of 1-50, 2-40, or 6-20 weight percent.

In one embodiment or in combination with any embodiment mentioned herein, the C1-C4 alkyl acetate is present in aqueous mixture 138 in an amount, by weight percent, that is within 25, 20, 15, 10, 5, or 2 weight percent of the solubility, by weight percent, of the C1-C4 alkyl acetate in water at 20° C.

The C1-C4 alkyl acetate may be one or both of methyl acetate and ethyl acetate. In one embodiment or in combination with any embodiment mentioned herein, when the C1-C4 alkyl acetate is ethyl acetate, ethyl acetate is present in aqueous mixture in an amount in the range of 2-25, 4-20, 6-15, or 8-10 weight percent. In one embodiment or in combination with any embodiment mentioned herein, when the C1-C4 alkyl acetate is methyl acetate, methyl acetate is present in aqueous mixture in an amount in the range of 5-50, 10-40, 15-35, or 20-30 weight percent.

In one particular embodiment, aqueous mixture 138 is formed from water 104, hydrocolloid 106, surfactant 108, and a C1-C4 alkyl acetate. In such an embodiment, water is present in aqueous mixture 138 in an amount in the range of 70-95, 75-95, 80-95, 85-95, or 87.5-92.5 weight percent, the hydrocolloid is present in aqueous mixture 138 in an amount in the range of 0.01-5, 0.1-4, 0.5-3, 0.6-2, 0.7-1, or 0.8-0.9 weight percent, the surfactant is present in aqueous mixture 138 in an amount in the range of 0.05-5, 0.1-5, 0.1-4, 0.5-3, 0.6-2, 0.7-1, or 0.8-1 weight percent, and the C1-C4 alkyl acetate is present in aqueous mixture 138 in an amount in the range of 2-40, 3-35, 4-30, 5-25, 5-20, 5-15, 5-10, or 6-10 weight percent.

In one embodiment or in combination with any embodiment mentioned herein, water 104, hydrocolloid 106, surfactant 108, and optionally a C1-C4 alkyl acetate are combined at unit 142 to produce aqueous mixture 138. In one embodiment or in combination with any embodiment mentioned herein, these components are mixed at a temperature of at least 15, 20, 25, 30, 35, 40, 45, or 50° C. and/or not more than 100, 75, 50, 40, 35, 30, 25, or 20° C., for a duration of at least 1, 2, 5, 10, 15, 20, 25, 30, 45, 60, 120, or 240 minutes and/or for as long as is needed to produce a substantially homogeneous mixture, depending on the viscosity of the hydrocolloid used.

### Emulsion/Dispersion Formation

Once formed, CE dope 136 and aqueous mixture 138 may be combined at unit 144 to produce an emulsion and/or a dispersion, as described above.

In one embodiment or in combination with any embodiment mentioned herein, a ratio of CE dope 136 to aqueous mixture 138 combined at unit 144 to form the initial emulsion is at least 0.05:1 to 10:1, 0.1:1 to 5:1, 0.2:1 to 2:1, or 0.4:1 to 0.8:1.

In one embodiment or in combination with any embodiment mentioned herein, the initial emulsion comprises water in an amount of at least 10, 20, 30, 40, 50, or 60 weight percent and/or not more than 90, 80, 70, 60, 50, or 40 weight percent.

Once combined, the initial emulsion is converted into a pre-hardened dispersion including the solid phase and the liquid phase. This conversion may be carried out by at least one of shearing, spraying (ultrasonic or electro), and membrane emulsion.

In one embodiment or in combination with any embodiment mentioned herein, the combined CE dope and aqueous mixture is recirculated through a high shear mixer to disperse the solid phase within the liquid phase, and to facilitate hardening of the solid phase to produce the initial microparticles. This shearing may be performed as CE dope 136 and aqueous mixture 138 are fed to unit 144 and/or may be performed after predetermined quantities of CE dope 136 and aqueous mixture 138 are within unit 144.

In one embodiment or in combination with any embodiment mentioned herein, the combined CE dope and aqueous mixture is recirculated through the high shear mixer for at least 1, 2, 3, 4, or 5 residence times and/or for not more than 20, 15, 10, 9, or 8 residence times based on the total volume of the high shear mixer used. In addition, the high shear mixing is performed for a duration of at least 1, 2, 5, 10, 15, 20, 25, 30, 45, 60, 120, or 240 minutes and/or for as long as is needed to recirculate the volume of the mixture for the predetermined number of residence times.

To produce initial microparticles that will form the CE microparticles as described herein, agitation of the combined CE dope and aqueous mixture is performed at unit 144. The agitation performed at unit 144 may be quantified by at least one of the following: (1) impeller tip speed, (2) impeller Reynolds number, and (3) power to mass ratio. In one embodiment or in combination with any embodiment mentioned herein, the high shear mixing is performed at an impeller tip speed of at least 25, 50, 75, 100, 150, 200, 250, or 300 cm/s and/or not more than 1000, 500, 400, 300, 200, or 100 cm/s. In one embodiment or in combination with any embodiment mentioned herein, the high shear mixing is performed at an impeller Reynolds number of at least 500, 1000, 1500, 2000, 3000, 4000, 5000, or 6000 and/or not more than 15000, 10000, 8000, 6000, 5000, 4000, or 3000. In one embodiment or in combination with any embodiment mentioned herein, the high shear mixing is performed at a power to mass ratio of at least 0.01, 0.02, 0.03, 0.1, 0.5, 1.0, 1.5, 2.0, 2.5 or 3.0 and/or not more than 10.0, 7.5, 5.0, 4.0, 3.0, 2.5, 2.0, 1.5, 1.0 0.5, or 0.1.

As used herein, terms such as “solid,” “solid phase,” “particles,” and “microparticles” refer to semi-solid materials that do not lose their discrete nature (i.e., flowing together) when an aqueous/continuous phase surrounding the material is removed.

Hardening with Extractant

A pre-hardened dispersion 150 formed at unit 144, and including the initial microparticles (i.e., solid phase), may be channeled to a hardening unit 152 to convert the initial particles to hardened CE microparticles 112. At unit 152, the initial microparticles contained within pre-hardened dispersion 150 are contacted with an extractant 154 (i.e., a drowning liquid) to produce a hardened dispersion 156. That is, the contacting step facilitates de-solventizing and hardening the initial microparticles into CE microparticles. In one embodiment or in combination with any embodiment mentioned herein, the extractant is water. Alternatively, the extractant may be methanol, ethanol, and combinations thereof.

In one embodiment or in combination with any embodiment mentioned herein, the pre-hardened dispersion and the extractant are combined at unit 152 at an extractant-to-dispersion weight ratio of at least 0.5:1, 1:1, 1.5:1, 2:1, or 1.5:1 and/or not more than 10:1, 8:1, 6:1, 4:1, or 3:1.

In one embodiment or in combination with any embodiment mentioned herein, the weight ratio of extractant to pre-hardened particles used in the contacting step is at least 2:1, 5:1, 10:1, 20:1. 30:1, or 40:1 and/or not more than 200:1, 100:1, 80:1. 60:1, or 50:1

As a result, in one embodiment or in combination with any embodiment mentioned herein, hardened dispersion 156 has water in one or more of the following amounts: (1) at least 25, 50, 60, 70, 80, 85, or 90 weight percent; (2) not more than 99, 97.5, 95, 92.5, 90, 80, 70, 60, or 50 weight percent; and (3) in the range of 50-99, 70-95, or 80-92.5 weight percent.

In one embodiment or in combination with any embodiment mentioned herein, hardened dispersion 156 has water in a weight concentration that is at least 1.5, 2, 2.5, 3, 3.5, 4, 4.5, 5 times greater than and/or not more than 100, 70, 50, 25, 15, 10, 5, or 2.5 times greater than the weight concentration of water in the initial emulsion.

In one embodiment or in combination with any embodiment mentioned herein, hardening of the microparticles is carried out under agitation. The agitation performed at unit 152 may be quantified by at least one of the following: (1) impeller tip speed, (2) impeller Reynolds number, and (3) power to mass ratio. In one embodiment or in combination with any embodiment mentioned herein, the converting/hardening is performed at an impeller tip speed of at least 25, 50, 75, 100, 150, 200, 250, or 300 cm/s and/or not more than 1000, 500, 400, 300, 200, or 100 cm/s. In one embodiment or in combination with any embodiment mentioned herein, the converting/hardening is performed at an impeller Reynolds number of at least 500, 1000, 1500, 2000, 3000, 4000, 5000, or 6000 and/or not more than 15000, 10000, 8000, 6000, 5000, 4000, or 3000. In one embodiment or in combination with any embodiment mentioned herein, the converting/hardening is performed at a power to mass ratio of at least 0.01, 0.02, 0.03, 0.1, 0.5, 1.0, 1.5, 2.0, 2.5 or 3.0 and/or not more than 10.0, 7.5, 5.0, 4.0, 3.0, 2.5, 2.0, 1.5, 1.0 0.5, or 0.1.

In addition, the converting/hardening is performed for a time period of at least 0.1, 0.5, 1, 2, 4, 6, 8, 10, or 20 minutes and/or not more than 12, 8, 6, 4, or 2 hours, and at a temperature of at least 0, 5, or 10° C. and/or not more than 100, 75, 50, or 25° C.

In one embodiment or in combination with any embodiment mentioned herein, the converting/hardening is performed in a single vessel, or in multiple vessels. Multiple vessels may be needed based on the output of CE microparticles to be produced and the volumetric capacity of available vessels used for the hardening. In embodiments where multiple vessels are used, a first portion of pre-hardened dispersion 150 may be received at a first hardening unit, and a second portion of pre-hardened dispersion may be received at a second hardening unit. Flow communication may then be provided between the separate hardening units to enhance mass transfer such that desolventization of the initial microparticles is accelerated.

In one embodiment or in combination with any embodiment mentioned herein, pre-hardened dispersion 150 has a solids content of at least 0.5, 1, 2, 3, or 4 and/or not more than 40, 30, 20, 10, or 6 weight percent.

As a result of the hardening, in one embodiment or in combination with any embodiment mentioned herein, hardened dispersion 156 has a solids content of at least 0.05, 0.1, 0.5, or 1 weight percent and/or not more than 20, 10, 5, 2, or 1 weight percent

In one embodiment or in combination with any embodiment mentioned herein, the solids content of the pre-hardened dispersion is at least 1.5, 2, 3, or 4 and/or not more than 20, 10, 8, or 6 times greater than the solids content of the hardened dispersion. In other words, at unit 152, solvent is extracted from the initial microparticles to produce hardened dispersion 156 including hardened CE microparticles 112 and a solvent-laden drowning liquid.

### CE Microparticle Isolation/Separation

CE microparticles 112 may be isolated and recovered from hardened dispersion 156 using any suitable technique at unit 158. In one embodiment or in combination with any embodiment mentioned herein, the isolating may be performed by at least one, at least two, or all three of the following: (1) flashing one or more liquid components away from the hardened CE microparticles; (2) filtering the hardened CE microparticles away from one or more liquid components; and (3) centrifuging, redispersing, and drying the hardened microparticles.

Solids processing and/or CE microparticle isolation may be performed in a single unit, as illustrated in FIG. 1, or may be performed in multiple units, as illustrated in FIG. 2. Referring to FIG. 2, a wet solids stream 160 and a separated mother liquor stream 162 are discharged from unit 158. Wet solids stream 160 contains the hardened CE microparticles and residual liquid. In one embodiment or in combination with any embodiment mentioned herein, wet solids stream 160 has a solids content of at least 10, 20, 25, 30, 35, 40, or 45 weight percent and/or not more than 60, 65, 50, 45, or 40 weight percent. This enables wet solids stream 160 to be conveyed to downstream units with reduced clogging and processing concerns.

Wet solids stream 160 may then, optionally, be processed in a wash unit 164, and a second solid/liquid separation unit 166. At unit 164, the hardened CE microparticles are washed with water 122 to produce a washed solids stream 168. Washed solids stream 168 is then processed at unit 166 by at least one, at least two, or all three of the following: (1) flashing one or more liquid components away from the hardened CE microparticles; (2) filtering the hardened CE microparticles away from one or more liquid components; and (3) centrifuging, redispersing, and drying the hardened microparticles. This second solid/liquid separation step facilitates reducing the solvent content in the liquid around the hardened CE microparticles.

A wash liquor 130 may be recovered from the second solid/liquid separation step and then recycled for use as at least a portion of the aqueous mixture in unit 142 and/or for use as at least a portion of the drowning liquid in unit 152.

In one embodiment or in combination with any embodiment mentioned herein, at least 1, 5, or 10 weight percent and/or not more than 90, 50, 20, or 10 weight percent of aqueous mixture 138 is the recycled wash liquor.

In one embodiment or in combination with any embodiment mentioned herein, at least 1, 5, or 10 weight percent and/or not more than 90, 50, 20, or 10 weight percent of drowning liquid 154 used in unit 152 is the recycled wash liquor.

A wet solids stream 172 discharged from unit 166 is then received at a drying unit 170. In one embodiment or in combination with any embodiment mentioned herein, drying is performed at unit 170 under agitation and with the addition of heat. Such agitation facilitates reducing agglomeration of the recovered CE microparticles 112. Properties of the recovered CE microparticles 112 are described in more detail below.

In one embodiment or in combination with any embodiment mentioned herein, drying unit 170 is a rotary cone dryer.

### Liquids Processing and Recycle

Separated mother liquor stream 162 discharged from unit 164 may be processed to recover water and/or solvent. The recovered water and/or solvent may then be recycled to one or more of the units shown in FIG. 2 to enhance the economic efficiency of the microparticles formation processes described herein.

The liquid processing may be performed in a single unit, as illustrated in FIG. 1, or may be performed in multiple units, as illustrated in FIG. 2. Referring to FIG. 2, mother liquor stream 162 contains at least a portion of the water and at least a portion of the solvent introduced at units 140 and/or 142, for example. Mother liquor stream 162 may also contain residual amounts of the hydrocolloid, surfactant, and any components used in the making of the mixed cellulose esters.

Mother liquor stream 162 may be heated 174 and then separated at unit 176 into at least two separate streams, such as solvent-enriched stream 134 and water-enriched (solvent-depleted) stream 132. Mother liquor stream 162 may be separated using any suitable technique at unit 176. In one embodiment or in combination with any embodiment mentioned herein, the liquids separation may be performed by distillation and the like.

In one embodiment or in combination with any embodiment mentioned herein, water-enriched stream 132 includes one or more of the following: (1) water; (2) a surfactant; (3) a hydrocolloid; and (4) a C1-C4 alkyl acetate.

Accordingly, water-enriched stream 132 may be cooled 181 and then recycled to one or more units illustrated in FIG. 2. For example, the composition of water-enriched stream 132 enables it to be recycled to at least one of unit 142 as stream 178 for use in the forming of aqueous mixture 138, and/or to unit 152 as stream 180 for use in de-solventizing the initial microparticles, thereby reducing water usage required for such processes. Any excess not required by these processes may be purged from the system and subjected to wastewater treatment, for example.

In one embodiment or in combination with any embodiment mentioned herein, the ratio of recycled water in stream 180 used in the hardening step to recycled water in stream 178 used in forming aqueous mixture 138 is at least 1:1, 1.5:1, 2:1, 3:1, 4:1 and/or not more than 20:1, 10:1, 8:1, or 6:1.

In one embodiment or in combination with any embodiment mentioned herein, the ratio of recycled water in stream 180 used in the hardening step to purge water is at least 1:1, 1.5:1, 2:1, 3:1, 4:1 and/or not more than 20:1, 10:1, 8:1, or 6:1.

In one embodiment or in combination with any embodiment mentioned herein, at least 75, 90, 95, 98, 99, or 100 weight percent of the extractant used in unit 152 is recycled water recovered downstream from unit 152, such as water contained in water-enriched stream 132 that is recycled to unit 152.

In one embodiment or in combination with any embodiment mentioned herein, fresh water 154 is added to unit 152 for use in the hardening step.

In one embodiment or in combination with any embodiment mentioned herein, wherein the ratio of the total amount of added fresh water to purge water is at least 0.25:1 0.5:1, 0.75:1, or 0.9:1 and/or not more than 4:1, 2:1, 1.5:1, 1.25:1, or 1.1:1.

In one embodiment or in combination with any embodiment mentioned herein, solvent-enriched stream 134 includes one or more of: (1) a C1-C4 alkyl acetate; (2) a C1-C4 alcohol; and (3) water.

Accordingly, solvent-enriched stream 134 may be recycled to one or more units illustrated in FIG. 2. For example, the composition of solvent-enriched stream 134 enables it to be recycled to at least one of unit 140 for use as a portion of the solvent in the forming of CE dope 136, and/or to unit 142 for use in the forming of aqueous mixture 138. Any excess not required by these processes may be purged from the system.

In one embodiment or in combination with any embodiment mentioned herein, at least 75, 90, 95, 98, 99, or 100 weight percent of the solvent used in unit 140 used to form CE dope 136 is recycled solvent 146.

In one embodiment or in combination with any embodiment mentioned herein, fresh water 104 is added to unit 142 for use in forming aqueous mixture 138.

In one embodiment or in combination with any embodiment mentioned herein, the ratio of the combined amount of water in streams 178 and 180 to the total amount of added fresh water 104 in unit 142 is at least 2:1, 4:1, 6:1, or 8:1.

In one embodiment or in combination with any embodiment mentioned herein, aqueous mixture 138 includes an azeotrope of water and C1-C4 alkyl acetate, and/or of water and C1-C4 alcohol, derived from water-enriched stream 132.

Hydroxyl is generally a strong hydrogen bonder. Thus, the opportunity for hydrogen bonding increases as the DS_{OH} of a given substance increases. The cosolvent alcohol of the solvent system described herein is also a strong hydrogen bonder. Surprisingly however, the ability of the cosolvent alcohol to hydrogen bond was found to decrease as the number of carbons (i.e., C1-C4) increases. Accordingly, lower carbon number binary solvent systems that did not contain water were found to be capable of dissolving cellulose esters having a DS_{OH} greater than a biodegradability threshold (e.g., DS_{OH} of greater than 0.8). In contrast, higher carbon number binary solvent systems that did not contain water were found to not be capable of dissolving cellulose esters having a DS_{OH} greater than the same biodegradability threshold. Accordingly, it has been found that the presence of a strong hydrogen bonder (i.e., water) with essentially no steric hindrance in solvent systems containing higher carbon number alcohols facilitates dissolution of the mixed CEs described herein in the solvent systems.

In one embodiment or in combination with any embodiment mentioned herein, recycled solvent 146 contains water in an amount of at least 1, 2, 4, 6, or 8 weight percent and/or not more than 50, 40, 30, 20, or 10 weight percent.

In one embodiment or in combination with any embodiment mentioned herein, recycled solvent 146 contains at least one azeotrope, wherein the azeotrope contains water and another component.

In one embodiment or in combination with any embodiment mentioned herein, the azeotrope may be in recycled solvent 146 may be a water/alcohol azeotrope, a water/alkyl acetate azeotrope, or both a water/alcohol azeotrope and a water/alkyl acetate azeotrope.

In one embodiment or in combination with any embodiment mentioned herein, recycled solvent 146 contains a plurality of azeotropes. In one embodiment or in combination with any embodiment mentioned herein, the plurality of azeotropes include a plurality of binary azeotropes. In one embodiment or in combination with any embodiment mentioned herein, the components of the solvent system are selected such that recycled solvent 146 does not contain any ternary azeotropes, thereby simplifying recovery and recycle of said solvent.

In one embodiment or in combination with any embodiment mentioned herein, solvent-enriched stream 134 contains less than 10 weight percent, less than 1 weight percent, less than 0.1 weight percent, or 0.0 weight percent of a ternary azeotrope.

In one embodiment or in combination with any embodiment mentioned herein, recycled solvent 146 contains three total binary azeotropes, such as a water/alcohol azeotrope, a water/alkyl acetate azeotrope, and an alcohol/alkyl acetate azeotrope.

### ADDITIONAL EMBODIMENTS

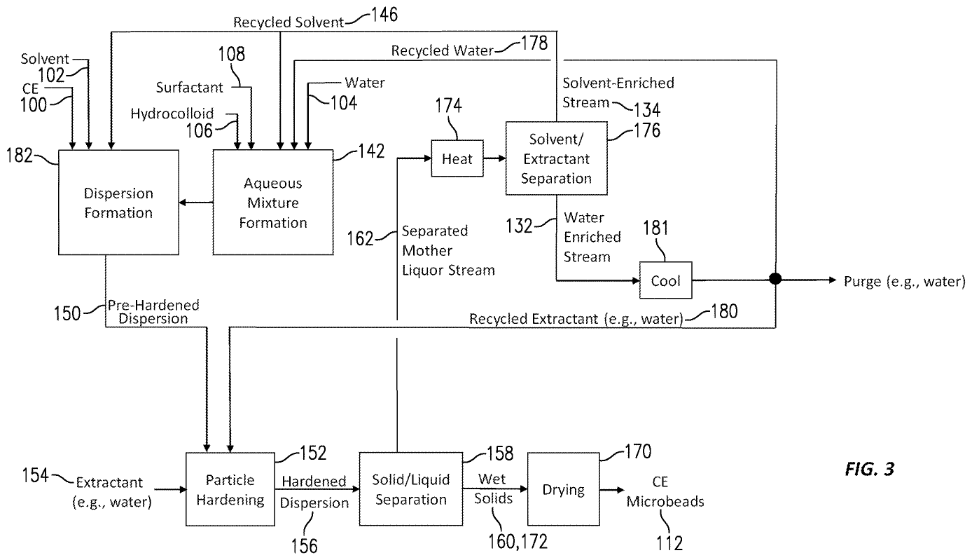

Referring now to FIG. 3, CE microparticles 112 are produced by serialized particle formation. In the example embodiment, particle formation begins at unit 142, in which water 104, hydrocolloid 106, surfactant 108, and, in some embodiments, recycled solvent 146 derived from solvent-enriched stream 134 is combined to form aqueous mixture 138.

Aqueous mixture 138 is discharged from unit 142 and received at a dispersion formation unit 182. At unit 182, aqueous mixture 138 is combined with CE 100, solvent 102, and, optionally, recycled solvent 146 derived from solvent-enriched stream 134. Thus, rather than forming CE dope 136 and aqueous mixture 138 in separate units, aqueous mixture 138, CE 100, and solvent 102 are combined in a common unit to form the initial emulsion.

In one embodiment or in combination with any embodiment mentioned herein, aqueous mixture 138, CE 100, and solvent 102 are agitated in unit 182 the combined CE dope and aqueous mixture is recirculated through a high shear mixer to disperse the solid phase within the liquid phase of the initial emulsion, and to facilitate hardening of the solid phase to produce the initial microparticles.

To produce the initial microparticles, agitation of the combined aqueous mixture 138, CE 100, and solvent 102 is performed at unit 182. The agitation performed at unit 182 may be quantified by at least one of the following: (1) impeller tip speed, (2) impeller Reynolds number, and (3) power to mass ratio. In one embodiment or in combination with any embodiment mentioned herein, the high shear mixing is performed at an impeller tip speed of at least 25, 50, 75, 100, 150, 200, 250, or 300 cm/s and/or not more than 1000, 500, 400, 300, 200, or 100 cm/s. In one embodiment or in combination with any embodiment mentioned herein, the high shear mixing is performed at an impeller Reynolds number of at least 500, 1000, 1500, 2000, 3000, 4000, 5000, or 6000 and/or not more than 15000, 10000, 8000, 6000, 5000, 4000, or 3000. In one embodiment or in combination with any embodiment mentioned herein, the high shear mixing is performed at a power to mass ratio of at least 0.01, 0.02, 0.03, 0.1, 0.5, 1.0, 1.5, 2.0, 2.5 or 3.0 and/or not more than 10.0, 7.5, 5.0, 4.0, 3.0, 2.5, 2.0, 1.5, 1.0 0.5, or 0.1.

Pre-hardened dispersion 150 is then discharged from unit 182 and hardened CE microparticles 112 recovered therefrom as described above.

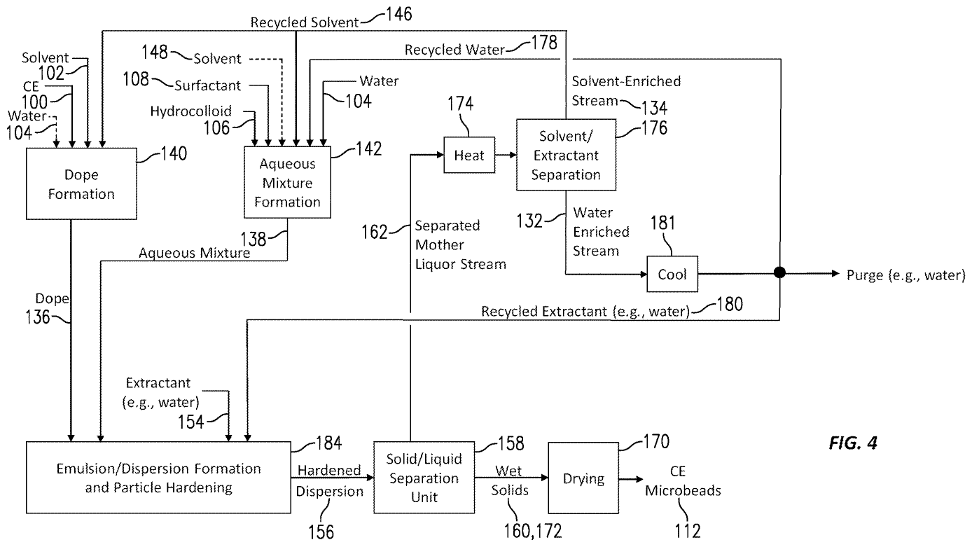

Referring to FIG. 4, CE microparticles 112 are produced by separated dope and aqueous mixture formation, and combined emulsion/dispersion formation and particle hardening. In the example embodiment, particle formation begins as illustrated in FIG. 2, wherein CE dope 136 and aqueous mixture 138 are formed in separate units.

In FIG. 4 however, CE dope 136 and aqueous mixture 138 are fed to a common emulsion/dispersion formation and particle hardening unit 184. At unit 184, CE dope 136 and aqueous mixture 138 are combined and agitated to form the initial emulsion as described herein. Once the initial emulsion is formed, extractant 154 is fed directly to unit 184 to perform de-solventization of the initial microparticles.

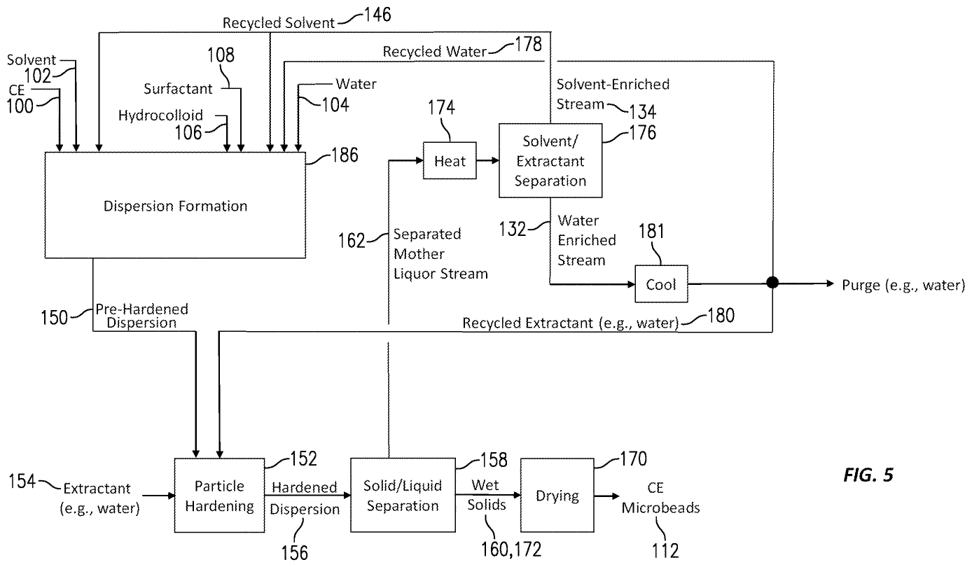

Referring to FIG. 5, CE microparticles 112 are produced by combined emulsion/dispersion formation. In the example embodiment, particle formation begins by combining all of CE 100, solvent 102, water 104, hydrocolloid 106, surfactant 108, and, optionally, recycled solvent 146 and recycled water 178 in a common unit 186. This mixture is agitated as described herein to produce pre-hardened dispersion 150 containing initial microparticles. Pre-hardened dispersion is received at unit 152 to de-solventize the initial microparticles as described herein.

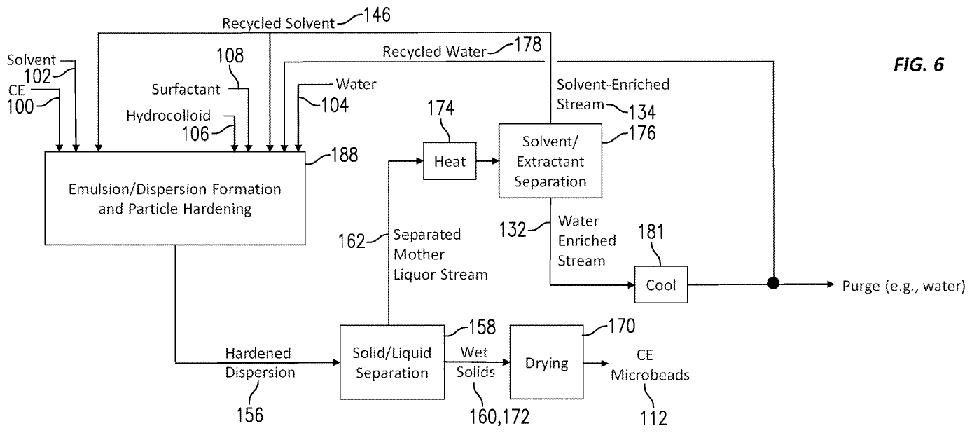

Referring to FIG. 6, CE microparticles 112 are produced by combined emulsion/dispersion formation and particle hardening. In the example embodiment, particle formation begins by combining all of CE 100, solvent 102, water 104, hydrocolloid 106, surfactant 108, and, optionally, recycled solvent 146 and recycled water 178 in a common unit 188. At unit 188, these components are combined and agitated to form the initial emulsion as described herein. Once the initial emulsion is formed, extractant 154 is fed directly to unit 188 to perform de-solventization of the initial microparticles.

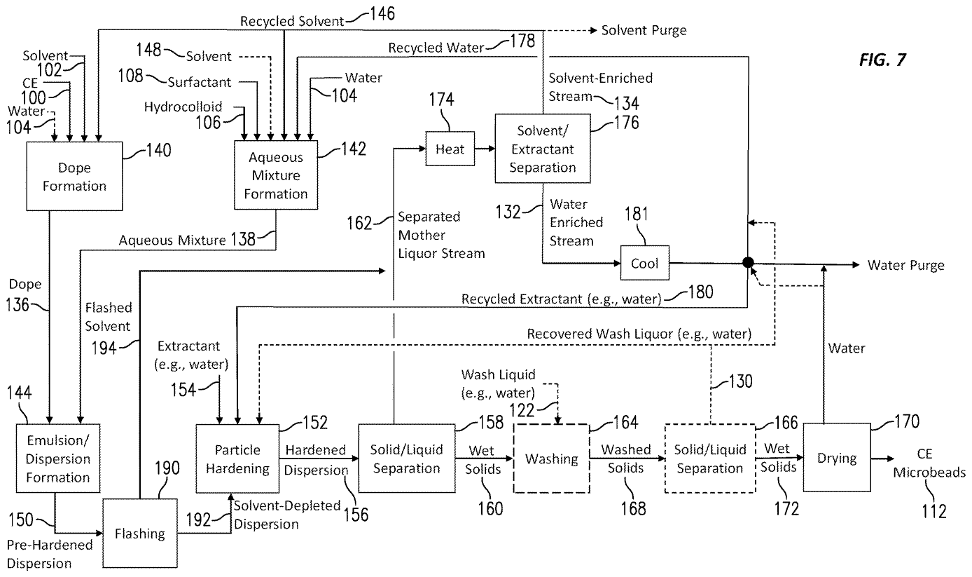

Referring to FIG. 7, CE microparticles 112 are produced by solvent flashing prior to particle hardening. In the example embodiment, pre-hardened dispersion 150 discharged from unit 144 is received at a flash unit 190, rather than at particle hardening unit 152. At unit 144, at least some of the solvent of pre-hardened dispersion 150 is removed from the liquid phase thereof, to thereby form a solvent-depleted dispersion 192 having a reduced solvent content. Solvent-depleted dispersion 192 is received at unit 152 to harden the pre-hardened microparticles contained therein, as described above. A flashed solvent stream 194 discharged from unit 190 may be channeled to unit 176 to perform liquids processing and recycle thereof.

In one embodiment or in combination with any embodiment mentioned herein, the removing at unit 144 is performed pervaporation, crossflow membrane filtration (ultrafiltration or nanofiltration), flash pot, spray pot, or wiped film evaporation.

In one embodiment or in combination with any embodiment mentioned herein, the removing at unit 144 reduces the solvent in said dispersion by at least 30 percent, at least 50 percent, at least 75 percent, at least 90 percent, between 30 and 90 percent, or between 50 and 75 percent by weight.

In one embodiment or in combination with any embodiment mentioned herein, solvent-depleted dispersion 192 has a solids content of at least 3, 4, 5, or 6 and/or not more than 40, 30, 20, or 10 weight percent.

In one embodiment or in combination with any embodiment mentioned herein, a volume ratio of drowning liquid to said dispersion used in unit 152 is less than 2.5:1, 2:1, 1.75:1, 1.5:1, 1.25:1, or 1:1.

### Hardened CE Microparticles

The hardened CE microparticles produced by the processes disclosed herein exhibit desirable tactile and/or optical qualities, for example, making them desirable for use in personal care products, cosmetics, and the like. As used herein, the terms “beads,” “microparticles” or “CE microparticles” may be used interchangeably with the term “hardened CE microparticles,” with the understanding that “hardened CE microparticles” are made via wet solvent/emulsion processes and that microbeads can also be produced via mechanical milling processes (e.g., jet milling) from larger CE form factors, including dry milling or size reduction processes (as described herein).

In one embodiment or in combination with any embodiment mentioned herein, CE microparticles are produced at a rate of at least 50, 100, 250, 500, 1000, 2500, 5000, or 10000 kg/day and/or not more than 100000, 75000, or 50000 kg/day. To achieve these production rates, CE, solvent, and/or water is provided within the system at one or more of the following corresponding rates.

In one embodiment or in combination with any embodiment mentioned herein, CE is provided at unit 140 at a rate of at least 50, 100, 250, 500, 1000, 2500, 5000, or 10000 kg/day and/or not more than 100000, 75000, or 50000 kg/day.

In one embodiment or in combination with any embodiment mentioned herein, solvent is provided at unit 140 at a rate of at least at least 50, 100, 250, 500, 1000, 2500, 5000, or 10000 kg/day and/or not more than 100000, 75000, or 50000 kg/day.

In one embodiment or in combination with any embodiment mentioned herein, water is provided unit 152 at a rate of at least at least 500, 1000, 2500, 5000, 10000, 25000, 50000, or 100000 kg/day and/or not more than 1000000, 750000, or 500000 kg/day.

In one embodiment or in combination with any embodiment mentioned herein, the hardened CE microparticles have a hardness at 20° C. that is greater than the hardness at 20° C. of the initial microparticles contained within pre-hardened dispersion 150 and/or formed in any of the processes described herein.

In one embodiment or in combination with any embodiment mentioned herein, the hardened CE microparticles have a hardness of at least 1.1, 1.25, 1.5, 1.75, or 2 times greater than the hardness of the initial microparticles.

In one embodiment or in combination with any embodiment mentioned herein, the hardened CE microparticles have a solvent content of less than 100, 50, 25, or 10 ppm.

In one embodiment or in combination with any embodiment mentioned herein, the hardened CE microparticles have a solvent content of less than the solvent content of the initial microparticles.

In one embodiment or in combination with any embodiment mentioned herein, the hardened CE microparticles have a solvent content of less than 0.99, 0.95, 0.9, 0.8, 0.7, 0.6, or 0.5 of the solvent content of the initial microparticles.

In one embodiment or in combination with any embodiment mentioned herein, the CE microparticles have a D50 volume-based particle size that is within 50, 25, 15, 10, 5, or 2 percent of the D50 particle size of the initial microparticles.

In one embodiment or in combination with any embodiment mentioned herein, the CE microparticles have a D50 volume-based particle size of less than 0.99, 0.95, 0.9, 0.8, 0.7, 0.6, or 0.5 of the D50 volume-based particle size of the initial microparticles.

In one embodiment or in combination with any embodiment mentioned herein, the CE microparticles have a D50 volume-based particle size in the range of 1 to 100, 1 to 80, 1 to 70, 1 to 60, 1 to 50, 1 to 40, 1 to 35, 1 to 30, 1 to 25, 1 to 20, 1 to 15, 1 to 10, 2 to 100, 2 to 80, 2 to 70, 2 to 60, 2 to 50, 2 to 40, 2 to 35, 2 to 30, 2 to 25, 2 to 20, 2 to 15, 2 to 10, 3 to 100, 3 to 80, 3 to 70, 3 to 60, 3 to 50, 3 to 40, 3 to 35, 3 to 30, 3 to 25, 3 to 20, 3 to 15, 3 to 10, 5 to 100, 5 to 80, 5 to 70, 5 to 60, 5 to 50, 5 to 40, 5 to 35, 5 to 30, 5 to 25, 5 to 20, 5 to 15, 5 to 10, 10 to 100, 10 to 80, 10 to 70, 10 to 60, 10 to 50, 10 to 40, 10 to 35, 10 to 30, 10 to 25, 10 to 20, 10 to 15, 15 to 100, 15 to 80, 15 to 70, 15 to 60, 15 to 50, 15 to 40, 15 to 35, 15 to 30, 15 to 25, 15 to 20, 20 to 100, 20 to 80, 20 to 70, 20 to 60, 20 to 50, 20 to 40, 20 to 35, 20 to 30, 25 to 100, 25 to 80, 25 to 70, 25 to 60, 25 to 50, 25 to 40, 25 to 35, 25 to 30, 30 to 100, 30 to 80, 30 to 70, 30 to 60, 30 to 50, 30 to 40, or 30 to 35 microns. For example, the CE microparticles can have a D50 volume-based particle size of 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 microns.

As used herein, the term “D50 volume-based” means that 50% of the beads/microparticles have a maximum dimension that is less than or equal to the noted value (e.g., 10 microns), based on a volume basis. The D50 value may also be treated as the median particle size. To ensure that a representative D50 value is obtained, the sample size of the beads/microparticles should be at least 0.5 grams. The microparticle sample size is then dispersed and mixed in 1.5 ounces of isopropanol. Testing for D50 is performed via laser diffraction and computer algorithms using the Mie theory to generate a particle size distribution. One suitable particle size analyzer for determining D50 values is the Malvern Mastersizer 3000 from Malvern Panalytical. When using the Malvern Mastersizer, the obscuration rate may be set between 2% and 5% and sample measurement time is set for three seconds for both red and blue light measurements. The dispersed sample is added until the desired obscuration rate (~4%) is attained and then the measurements are carried out. After the first measurement, the sample is sonicated at 50% power for 120 seconds. Subsequently, after sonication, the dispersed sample is measured again once the light energy stabilizes (usually less than one minute).

In one embodiment or in combination with any embodiment mentioned herein, the CE microparticles have a D10 volume-based particle size of 0.5 to 20, 0.5 to 15, 0.5 to 12, 0.5 to 10, 0.5 to 5, 0.5 to 4, 0.5 to 3, 0.5 to 2, 0.5 to 1, 1 to 20, 1 to 15, 1 to 12, 1 to 5, 1 to 3, 2 to 20, 2 to 10, 2 to 5, 3 to 20, 3 to 15, 3 to 10, 4 to 20, 4 to 15, 4 to 10, 5 to 20, 5 to 15, 5 to 10, 10 to 20, or 10 to 15 microns. For example, the CE microparticles can have a D10 volume-based particle size of 0.5, 1, 2, 3, 4, 5, 6, 7, 8, 9, 10, 11, 12, 13, 14, 15, 16, 17, 18, 19, or 20 microns.

As used herein, the term “D10 volume-based” means that 10% of the beads/microparticles have a maximum dimension that is less than or equal to the noted value (e.g., 10 microns), based on a volume basis. To ensure that a representative D10 value is obtained, the sample size of the beads/microparticles should be at least 0.5 grams. The microparticle sample size is then dispersed and mixed in 1.5 ounces of isopropanol. Testing for D10 is performed via laser diffraction and computer algorithms using the Mie theory to generate a particle size distribution. One suitable particle size analyzer for determining D10 values is the Malvern Mastersizer 3000 from Malvern Panalytical. When using the Malvern Mastersizer, the obscuration rate may be set between 2% and 5% and sample measurement time is set for three seconds for both red and blue light measurements. The dispersed sample is added until the desired obscuration rate (~4%) is attained and then the measurements are carried out. After the first measurement, the sample is sonicated at 50% power for 120 seconds. Subsequently, after sonication, the dispersed sample is measured again once the light energy stabilizes (usually less than one minute).

In one embodiment or in combination with any embodiment mentioned herein, the CE microparticles have a D90 volume-based particle size in the range of 1 to 100, 1 to 80, 1 to 70, 1 to 60, 1 to 50, 1 to 40, 1 to 35, 1 to 30, 1 to 25, 1 to 20, 1 to 15, 1 to 10, 5 to 100, 5 to 80, 5 to 70, 5 to 60, 5 to 50, 5 to 40, 5 to 35, 5 to 30, 5 to 25, 5 to 20, 5 to 15, 5 to 10, 10 to 100, 10 to 80, 10 to 70, 10 to 60, 10 to 50, 10 to 40, 10 to 35, 10 to 30, 10 to 25, 10 to 20, 10 to 15, 15 to 100, 15 to 80, 15 to 70, 15 to 60, 15 to 50, 15 to 40, 15 to 35, 15 to 30, 15 to 25, 15 to 20, 20 to 100, 20 to 80, 20 to 70, 20 to 60, 20 to 50, 20 to 40, 20 to 35, 20 to 30, 25 to 100, 25 to 80, 25 to 70, 25 to 60, 25 to 50, 25 to 40, 25 to 35, 25 to 30, 30 to 100, 30 to 80, 30 to 70, 30 to 60, 30 to 50, 30 to 40, or 30 to 35 microns. For example, the CE microparticles can have a D90 volume-based particle size of 100, 90, 80, 70, 60, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, or 20 microns.

As used herein, the term “D90 volume-based” means that 90% of the beads/microparticles have a maximum dimension that is less than or equal to the noted value (e.g., 10 microns), based on a volume basis. To ensure that a representative D90 value is obtained, the sample size of the beads/microparticles should be at least 0.5 grams. The microparticle sample size is then dispersed and mixed in 1.5 ounces of isopropanol. Testing for D90 is performed via laser diffraction and computer algorithms using the Mie theory to generate a particle size distribution. One suitable particle size analyzer for determining D90 values is the Malvern Mastersizer 3000 from Malvern Panalytical. When using the Malvern Mastersizer, the obscuration rate may be set between 2% and 5% and sample measurement time is set for three seconds for both red and blue light measurements. The dispersed sample is added until the desired obscuration rate (~4%) is attained and then the measurements are carried out. After the first measurement, the sample is sonicated at 50% power for 120 seconds. Subsequently, after sonication, the dispersed sample is measured again once the light energy stabilizes (usually less than one minute).

In one embodiment or in combination with any embodiment mentioned herein, the CE microparticles have a D100 volume-based particle size in the range of 1 to 100, 1 to 80, 1 to 70, 1 to 60, 1 to 50, 1 to 40, 1 to 35, 1 to 30, 1 to 25, 1 to 20, 1 to 15, 1 to 10, 5 to 100, 5 to 80, 5 to 70, 5 to 60, 5 to 50, 5 to 40, 5 to 35, 5 to 30, 5 to 25, 5 to 20, 5 to 15, 5 to 10, 10 to 100, 10 to 80, 10 to 70, 10 to 60, 10 to 50, 10 to 40, 10 to 35, 10 to 30, 10 to 25, 10 to 20, 10 to 15, 15 to 100, 15 to 80, 15 to 70, 15 to 60, 15 to 50, 15 to 40, 15 to 35, 15 to 30, 15 to 25, 15 to 20, 20 to 100, 20 to 80, 20 to 70, 20 to 60, 20 to 50, 20 to 40, 20 to 35, 20 to 30, 25 to 100, 25 to 80, 25 to 70, 25 to 60, 25 to 50, 25 to 40, 25 to 35, 25 to 30, 30 to 100, 30 to 80, 30 to 70, 30 to 60, 30 to 50, 30 to 40, or 30 to 35 microns. For example, the CE microparticles can have a D100 volume-based particle size of 100, 90, 80, 70, 60, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, or 20 microns.

As used herein, the term “D100 volume-based” means that 100% of the beads/microparticles have a maximum dimension that is less than or equal to the noted value (e.g., 10 microns), based on a volume basis. To ensure that a representative D100 value is obtained, the sample size of the beads/microparticles should be at least 0.5 grams. The microparticle sample size is then dispersed and mixed in 1.5 ounces of isopropanol. Testing for D100 is performed via laser diffraction and computer algorithms using the Mie theory to generate a particle size distribution. One suitable particle size analyzer for determining D100 values is the Malvern Mastersizer 3000 from Malvern Panalytical. When using the Malvern Mastersizer, the obscuration rate may be set between 2% and 5% and sample measurement time is set for three seconds for both red and blue light measurements. The dispersed sample is added until the desired obscuration rate (~4%) is attained and then the measurements are carried out. After the first measurement, the sample is sonicated at 50% power for 120 seconds. Subsequently, after sonication, the dispersed sample is measured again once the light energy stabilizes (usually less than one minute).

In one embodiment or in combination with any embodiment mentioned herein, the CE microparticles have a D[4,3] mean particle size in the range of 1 to 100, 1 to 80, 1 to 70, 1 to 60, 1 to 50, 1 to 40, 1 to 35, 1 to 30, 1 to 25, 1 to 20, 1 to 15, 1 to 10, 1 to 8, 1 to 6, 1 to 5, 2 to 40, 2 to 35, 2 to 30, 2 to 25, 2 to 20, 2 to 15, 2 to 10, 2 to 8, 2 to 6, 2 to 5, 3 to 40, 3 to 35, 3 to 30, 3 to 25, 3 to 20, 3 to 15, 3 to 10, 3 to 8, 3 to 6, 3 to 5, 4 to 40, 4 to 35, 4 to 30, 4 to 25, 4 to 20, 4 to 15, 4 to 10, 4 to 8, 4 to 6, 5 to 100, 5 to 80, 5 to 70, 5 to 60, 5 to 50, 5 to 40, 5 to 35, 5 to 30, 5 to 25, 5 to 20, 5 to 15, 5 to 10, 10 to 100, 10 to 80, 10 to 70, 10 to 60, 10 to 50, 10 to 40, 10 to 35, 10 to 30, 10 to 25, 10 to 20, 10 to 15, 15 to 100, 15 to 80, 15 to 70, 15 to 60, 15 to 50, 15 to 40, 15 to 35, 15 to 30, 15 to 25, 15 to 20, 20 to 100, 20 to 80, 20 to 70, 20 to 60, 20 to 50, 20 to 40, 20 to 35, 20 to 30, 25 to 100, 25 to 80, 25 to 70, 25 to 60, 25 to 50, 25 to 40, 25 to 35, 25 to 30, 30 to 100, 30 to 80, 30 to 70, 30 to 60, 30 to 50, 30 to 40, or 30 to 35 microns. For example, the CE microparticles can have a D100 volume-based particle size of 100, 90, 80, 70, 60, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, or 20 microns.

As used herein, the term “D[4,3] mean particle size” means the D[4,3] mean as described in ASTM E 799 and also known as De Brouckere mean, which divides the sum of the fourth power of Di*percentage, divided by third power of the same calculation. To ensure that a representative D[4,3] value is obtained, the sample size of the beads/microparticles should be at least 0.5 grams. The microparticle sample size is then dispersed and mixed in 1.5 ounces of isopropanol. One suitable particle size analyzer for determining D100 values is the Malvern Mastersizer 3000 from Malvern Panalytical. When using the Malvern Mastersizer, the obscuration rate may be set between 2% and 5% and sample measurement time is set for three seconds for both red and blue light measurements. The dispersed sample is added until the desired obscuration rate (~4%) is attained and then the measurements are carried out. After the first measurement, the sample is sonicated at 50% power for 120 seconds. Subsequently, after sonication, the dispersed sample is measured again once the light energy stabilizes (usually less than one minute).

In one embodiment or in combination with any embodiment mentioned herein, the CE microparticles have an average sphericity of at least at least 10, 20, 30, 40, 50, 60, 70, 80, 90, 95, 95, 97, 98, or 99 percent. Additionally, or in the alternative, the CE microparticles can have an average sphericity of not more than 99, 95, 90, 80, 70, 60, 50, 40, or 30 percent. In certain embodiments, the CE microparticles have an average sphericity of in a range from 5 to 60, or 5 to 50, or 5 to 40, or 5 to 30, or 5 to 25, or 5 to 20, or 10 to 60, or 10 to 50, or 10 to 40, or 10 to 30, or 10 to 25, or 10 to 20 percent. In embodiments, the CE microparticles are produced via a mechanical size reduction process (as described herein), e.g., jet milling, and have an average sphericity of in a range from 5 to 40, or 5 to 30, or 5 to 25, or 5 to 20, or 10 to 40, or 10 to 30, or 10 to 25, or 10 to 20 percent.

In certain embodiments, the CE microparticles have an average sphericity of in a range from 70 to 100, or 70 to 90, or 70 to 80, or 75 to 100, or 75 to 90, or 75 to 80, or 80 to 100, or 80 to 90 percent. In embodiments, the CE microparticles are produced via a solvent or emulsion process (as described herein) and have an average sphericity of in a range from 70 to 100, or 70 to 90, or 70 to 80 percent.

Average sphericity is determined by: (1) obtaining a secondary emission/ETD detector scanning electron microscopy (SEM) image of a representative sample of at least 40 microparticles, (2) on the SEM image, selecting a square sample window centered at the center of the SEM that contains exactly 30 microparticles whose entire outer perimeters are clearly visible (i.e., not occluded), (3) measuring the maximum and minimum diameters (each extending through the particle's centroid and not necessarily perpendicular to one another) of the 30 clearly visible microparticles in the sample window, (4) for each of the 30 particles, dividing the minimum diameter by the maximum diameter and multiplying the result by 100% to obtain 30 individual particle sphericities, and (5) averaging the 30 individual particle sphericities to obtain the average sphericity. As used herein, the term “spherical” with regard to describing the shape of the CE microparticles means that the CE microparticles have an average sphericity of at least 70 percent. As used herein, the term “spheroidal” with regard to describing the shape of the CE microparticles means that the CE microparticles have an average sphericity of less than 70 percent.

In one embodiment or in combination with any embodiment mentioned herein, the CE microparticles exhibit a monomodal particle size distribution with a span of at least 0.5, 0.55, 0.6, 0.65, 0.7, 0.75, 0.8, 0.85, 0.9, 0.95, 1.0, 1.05, 1.1, 1.15, 1.2, 1.25, 1.3, or 1.4 and/or less than 3.0, 2.9, 2.8, 2.7, 2.6, 2.5, 2.4, 2.3, 2.2, 2.1, 2.0, 1.9, 1.8, 1.7, 1.6, or 1.5. In certain embodiments, the CE microparticles exhibit a monomodal particle size distribution with a span of 1.0 to 3.0, 1.0 to 2.5, 1.0 to 2.0, 1.0 to 1.8, 1.0 to 1.6, 1.2 to 3.0, 1.2 to 2.5, 1.2 to 2.0, 1.2 to 1.8, 1.2 to 1.6, 1.3 to 3.0, 1.3 to 2.5, 1.3 to 2.0, 1.3 to 1.8, or 1.3 to 1.6. As used herein, “monomodal particle size distribution” refers to a particle size distribution for a material that only has a single notable peak of size distribution. This is in contrast to multi-modal particle size distributions, which will have two or more peaks of particle size distributions. The “span” of the monomodal peak may be measured using the D10, D50, and D90 values of the particles using the following formula:

$$( {D}_{x} ( 90 ) - {D}_{x} ( 10 ) ) / {D}_{x} ( 50 ) ,$$

wherein “x” is the designated particle size.

In one embodiment or in combination with any embodiment mentioned herein, the CE microparticles have an average smoothness of at least 10, 20, 30, 40, 50, 60, 70, 80, 90, 95, 95, 97, 98, or 99 percent. Additionally, or in the alternative, the CE microparticles can have an average smoothness of not more than 99, 95, 90, 80, 70, 60, 50, 40, or 30 percent.

Average smoothness is determined by: (1) obtaining a secondary emission/EDT detector scanning electron microscopy (SEM) image of a representative sample of at least 20 microparticles, (2) on the SEM, selecting a square sample window centered at the center of the SEM that contains exactly 10 microparticles whose entire outer perimeters are clearly visible (i.e., not occluded), (3) binarizing the sample window by manual binarization with upper and lower thresholds chosen to match the exact shape of the darker regions of the particles, (4) for each of the 10 microparticles, selecting a square window at or near the center of the particle having length and width that are approximately ⅓ of the particle diameter (before binarizing the particle), (5) dividing the dark region area in the square window by the total area of the square window and multiplying the result by 100% to obtain 10 individual particle smoothness, and (5) averaging the 10 individual particle smoothness to obtain the average smoothness.

In one embodiment or in combination with any embodiment mentioned herein, the CE microparticles have an average BET surface area of at least 0.1, 0.5, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4. 6.5, 6.7, 6.8, 6.9, 7.0, 7.1, 7.2, 7.3, 7.4, 7.5, 7.6, 7.7, 7.8, 7.9, or 8.0 m²/g and/or not more than 100, 95, 90, 85, 80, 75, 70, 65, 60, 55, 50, 45, 40, 35, 30, 29, 28, 27, 26, 25, 24, 23, 22, 21, 20, 19, 18, 17, 16, 15, 14, 13, 12, 11, 10, 9, 8, 7, 6, 5, 4, 3, 2.5, 2, 1.9, 1.8, 1.7, 1.6, 1.5, 1.4, or 1.3 m²/g as measured according to ISO 9277 using a Micromeritics ASAP 2020 instrument and krypton gas.

In one embodiment or in combination with any embodiment mentioned herein, the CE microparticles have a BET average pore size of at least 25, 30, 35, 40, 45, 50, 55, 56, 57, 58, or 59 angstroms and/or less than 75, 70, 65, or 60 angstroms as measured according to ISO 9277 and ISO 15901-02 using a Micromeritics ASAP 2020 instrument and nitrogen gas.

In one embodiment or in combination with any embodiment mentioned herein, the CE microparticles have a BJH average pore size of at least 50, 60, 70, 80, 90, 100, 110, 120, 125, or 130 angstroms and/or less than 200, 190, 180, 170, 160, 150, 140, or 130 angstroms as measured according to ISO 15901-02 using a Micromeritics ASAP 2020 instrument and nitrogen gas.

In one embodiment or in combination with any embodiment mentioned herein, the CE microparticles have a BJH surface area of pores from 17 to 3,000 angstroms of at least 0.5, 1.0, 1.1, 1.2, 1.3, 1.4, or 1.5 and/or less than 2.5, 2.0, 1.9, 1.8, 1.7, 1.6, 1.5, 1.4, or 1.3 m²/g as measured according to ISO 15901-02 using a Micromeritics ASAP 2020 instrument and nitrogen gas.

In one embodiment or in combination with any embodiment mentioned herein, the CE microparticles have a BJH volume of pores from 17 to 3,000 angstroms of at least 0.001, 0.002, 0.003, or 0.004 and/or less than 0.1, 0.05, or 0.01 mL/g as measured according to ISO 15901-02 using a Micromeritics ASAP 2020 instrument and nitrogen gas.

In one embodiment or in combination with any embodiment mentioned herein, the CE microparticles have a true specific gravity of at least 0.3, 0.4, 0.5, 0.6, 0.7, 0.8, or 0.9 and/or not more than 1.5, 1.4, 1.3, 1.2, 1.1, 1.0, 0.9, 0.8, 0.7, or 0.6 as measured by JIS Z8807-1976.

In one embodiment or in combination with any embodiment mentioned herein, the CE microparticles have a bulk specific gravity of at least 0.2, 0.3, 0.4, 0.5, 0.6, 0.7, or 0.8 and/or not more than 1.4, 1.3, 1.2, 1.1, 1.0, 0.9, 0.8, 0.7, 0.6, or 0.5 as measured by JIS 1201-1.

In one embodiment or in combination with any embodiment mentioned herein, the CE microparticles have a polydispersity index of less than 0.8, 0.7, 0.6, 0.5, 0.4, or 0.3.

In one embodiment or in combination with any embodiment mentioned herein, the CE microparticles have a surfactant content of less than 200, 150, 100, 50, 20, 10, 7.5, 5, 2.5, or 1 ppmw.

In one embodiment or in combination with any embodiment mentioned herein, the CE microparticles have a plasticizer content of less than 200, 150, 100, 50, 20, 10, 7.5, 5, 2.5, or 1 ppmw.

In one embodiment or in combination with any embodiment mentioned herein, the CE microparticles have a butyric acid content of less than 500, 400, 300, 200, 100, 50, 20, 10, 7.5, 5, 2.5, or 1 ppmw.

In one embodiment or in combination with any embodiment mentioned herein, the CE microparticles have an acetic acid content of less than 500, 400, 300, 200, 100, 50, 20, 10, 7.5, 5, 2.5, or 1 ppmw.

In one embodiment or in combination with any embodiment mentioned herein, the CE microparticles have a propionic acid content of less than 500, 400, 300, 200, 100, 50, 20, 10, 7.5, 5, 2.5, or 1 ppmw.

The butyric acid, acetic acid, and propionic acid contents of the CE microparticles may be measured via gas chromatography (“GC”). Under one GC methodology, the butyric acid, acetic acid, and propionic acid contents may be measured by adding about 100 mg of the CE microparticles to a tared 4-dram vial, followed by the addition of an internal standard solution comprising nonane in a 90:10 mixture of dichloromethane/methanol. A magnetic stir bar is placed in the vial, and the sample is stirred for two hours. After stirring, 8.0 mL of n-heptane is added dropwise to precipitate the polymer, and then the sample is vortexed. Approximately 100 mg of the supernatant is transferred to a GC vial, along with 100 μL of pyridine and 450 μL of BSTFA. The samples are heated at 80° C. for 30 minutes and then cooled to room temperature before injection. Samples are chromatographed simultaneously on 100% dimethylpolysiloxane and 14% cyanopropyl-phenyl-methylpolysiloxane columns using temperature programming and flame ionization detection. Alternatively, a second GC methodology involves preparing samples by adding approximately 30 mg of CE microparticles to a tared GC vial, followed by 200 μL of an internal standard solution comprising decane in pyridine, and 1.0 mL of BSTFA. The vials re heated at 80° C. for 30 minutes and then cooled to room temperature before injection. Samples are then chromatographed simultaneously on 100% dimethylpolysiloxane and 6% cyanopropyl-phenyl-methylpolysiloxane columns using temperature programming and flame ionization detection.

In one embodiment or in combination with any embodiment mentioned herein, the CE microparticles have a sulfuric acid content of less than 500, 400, 300, 200, 100, 50, 20, 10, 7.5, 5, 2.5, or 1 ppmw. The sulfuric acid content of the CE microparticles may be measured by the following methodology. First, the tested sample is added to a titration cell and dissolved in a solvent for a total volume of 70 mL. Solvent blanks are also prepared for comparison purposes. The samples and blanks are then titrated with 0.05 N potassium hydroxide in methanol using an automatic titrator equipped with a combination glass potentiometric electrode. Acid number is calculated based on the sample weight and the KOH consumed in the sample minus the KOH consumed in the blank.

In one embodiment or in combination with any embodiment mentioned herein, the CE microparticles have a CE content of at least 50, 55, 60, 65, 70, 75, 80, 85, 90, 91, 92, 93, 94, 95, 96, 97, 98, or 99 weight percent of the mixed cellulose ester of the first aspect, the second aspect, and/or the third aspect, including any class or subclass of these aspects. Additionally, or in the alternative, the CE microparticles may have a CE content of less than 99.9, 99.5, 99, 98, 97, 96, 95, 94, 93, 92, 91, 90, 89, 88, 87, 86, or 85 weight percent of the mixed cellulose ester of the first aspect, the second aspect, and/or the third aspect, including any class or subclass of these aspects. In certain embodiments, the CE microparticles may consist essentially of the mixed cellulose ester of the first aspect, the second aspect, and/or the third aspect, including any class or subclass of these aspects.

In one embodiment or in combination with any embodiment mentioned herein, the CE microparticles may contain an additional biodegradable cellulose ester that is different from the mixed cellulose ester of the first aspect, the second aspect, and/or the third aspect. In such embodiments, this additional cellulose ester can be cellulose acetate, which exhibits at least 40% biodegradability, at least 45% biodegradability, or at least 50% biodegradability, or at least 55% biodegradability, at least 60% biodegradability, or at least 65% biodegradability, or at least 70% biodegradability, or at least 75% biodegradability, or at least 80% biodegradability, or at least 85% biodegradability, at 60 days according to at least one of the OECD 301B, OECD 301C, or OECD 301F test methods.

In one embodiment or in combination with any embodiment mentioned herein, the CE microparticles may contain at least 1, 2, 3, 4, 5, 6, 7, 8, 9, or 10 weight percent the additional biodegradable cellulose ester that is different from the mixed cellulose ester of the first aspect, the second aspect, and/or the third aspect. Additionally, or in the alternative, the CE microparticles may contain less than 50, 45, 40, 35, 30, 25, 20, 15, 10, or 5 weight percent of the additional biodegradable cellulose ester that is different from the mixed cellulose ester of the first aspect, the second aspect, and/or the third aspect.

In one embodiment or in combination with any embodiment mentioned herein, the CE microparticles have moisture content in one or more of the following amounts: (1) greater than 0 weight percent; (2) not more than 10, 9, 8, 7, 6, 5, 4, 3, 2, 1, or 0.05 weight percent; and (3) in the range of 0-10, 0-5, 0-4, 0-3, or 1.3 weight percent.

In one embodiment or in combination with any other embodiment mentioned herein, the CE microparticles exhibit at least 40% biodegradability, at least 45% biodegradability, or at least 50% biodegradability, or at least 55% biodegradability, at least 60% biodegradability, or at least 65% biodegradability, or at least 70% biodegradability, or at least 75% biodegradability, or at least 80% biodegradability, or at least 85% biodegradability, at 56 days according to at least one of the OECD 301B, OECD 301C, or OECD 301F test methods.

In one embodiment or in combination with any other embodiment mentioned herein, the CE microparticles exhibit at least 40% biodegradability, at least 45% biodegradability, or at least 50% biodegradability, or at least 55% biodegradability, at least 60% biodegradability, or at least 65% biodegradability, or at least 70% biodegradability, or at least 75% biodegradability, or at least 80% biodegradability, or at least 85% biodegradability, at 60 days according to at least one of the OECD 301B, OECD 301C, or OECD 301F test methods.

In one embodiment or in combination with any other embodiment mentioned herein, the CE microparticles exhibit an oil absorption of at least 30, 35, 40, 45, 50, 55, 60, 65, 70, 75, 80, 85, 90, 95, or 100 mL per 100 g (or alternatively g oil per 100 g microparticles) as measured using test method ASTM D281, wherein mineral oil is used instead of castor oil.

In one embodiment or in combination with any other embodiment mentioned herein, the CE microparticles exhibit a zeta potential of at least −95, at least −90, at least −85, at least −80, at least −75, at least −70, at least −65, at least −60, at least −55, at least −50, or at least −45 mV. Additionally, or in the alternative, the CE microparticles may exhibit a zeta potential of less than −5, less than −10, less than −15, less than −20, less than −25, less than −30, less than −35, less than −40, less than −45, less than −50, less than −55, less than-60, or less than −65 mV.

Zeta potential was measured by dispersing the microparticles in water by vortex mixing for 30 seconds. The microparticle concentration was controlled at 0.5 mg/ml. Zeta potential tests were done on a Zetasizer Nano series, model ZEN 3600 instrument from Malvern Panalytical with a sample cell DTS1070. A Smoluchowski model is then used for Zeta potential calculation.

In one embodiment or in combination with any other embodiment mentioned herein, the CE microparticles exhibit a haze transmission of at least 20, at least 25, at least 30, at least 35, at least 40, or at least 45 percent. Additionally, or in the alternative, the CE microparticles may exhibit a haze transmission of less than 90, less than 85, less than 80, less than 75, less than 70, less than 65, less than 60, less than 55, less than 50, less than 45, less than 40 percent. The haze transmission may be measured using a BYK Glossmeter and a BYK Haze Gard I by forming an aqueous emulsion comprising 5 weight percent of the biodegradable beads/microparticles.

The detailed procedure for measuring the haze follows. The W/O emulsion for measuring haze % was prepared using the following procedure. Two phases were used for preparing the emulsion. Phase A is made from water, magnesium sulfate heptahydrate (Merck), and Euxyl PE9010 (Ashland) at the following weight concentration, 59:2:1, respectively. Phase B is prepared with caprylic/capric triglycerides (Making Cosmertics), C12 15 alkyl benzoate (Making Cosmetics), Emullium Illustro (Gattefosse), bentone Gel ISD V (Elementis), microparticle powder at the following weight concentrations, 12.5:12.5:5:3:5, respectively. Phase A is prepared by mixing the listed components. Phase B is prepared without the microparticle powder by overhead stirring until all of the components are dissolved. Then Phase A is added to Phase B while stirring at 1000 rpm until fully mixed. Then the microparticle powder is added to the mixture while mixing at 1000 rpm for 5 minutes. The resulting mixture is homogenized with an Ultra Turax for 5 minutes at 10,000 rpm. The haze transmission of drawdown films (38 μm) after 5 min drying at 50° C. are measured using BYK Haze Gard I.

In one embodiment or in combination with any other embodiment mentioned herein, the CE microparticles exhibit a total transmission of at least 50, at least 60, at least 70, at least 75, at least 80, at least 85, at least 86, at least 87, at least 88, or at least 89 percent as measured using a BYK Haze-Gard I unit using the procedure disclosed herein.

### Cosmetic Formulations

The hardened CE microparticles produced by the processes disclosed herein (e.g., emulsion processes) or CE microparticles produced by physical size reduction processes (e.g., jet milling) may be used to produce a variety of cosmetic compositions. The cosmetic compositions may be produced by: (1) providing a plurality of the CE microparticles; (2) combining the CE beads/microparticles with one or more cosmetic additives to thereby form a pre-cosmetic mixture; and (3) forming the cosmetic composition from the pre-cosmetic mixture.

In one embodiment or in combination with any other embodiment mentioned herein, the cosmetic composition can comprise at least 0.1, 0.5, 1, 2, 3, 4, 5, 6, 7, 8, 9, 10, 11, 12, 13, 14, or 15 weight percent of the CE microparticles. Additionally, or in the alternative, the cosmetic composition can comprise less than 99, 90, 80, 70, 60, 50, 40, 30, 25, 20, 15, 10, or 5 weight percent of the CE microparticles. For example, the cosmetic composition can comprise 0.1 to 90, 0.1 to 50, 0.1 to 30, 0.1 to 20, 0.1 to 15, 0.1 to 10, 0.1 to 5, 1 to 90, 1 to 50, 1 to 30, 1 to 20, 1 to 15, 1 to 10, or 1 to 5 weight percent of the CE microparticles.

In one embodiment or in combination with any other embodiment mentioned herein, the cosmetic composition can be a foundation, a sunscreen, a lipstick, a mascara, an eye shadow, a lotion, a dry shampoo, a liquid shampoo, a body wash, a lotion, a hair conditioner, a skin moisturizer, a face wash, a tablet, a foot powder, a baby powder, a shaving cream, or a shaving gel.

In one embodiment or in combination with any other embodiment mentioned herein, the cosmetic composition can be a loose powder, a compacted powder, a gel, an emulsion, a liquid, or an aerosol.

In one embodiment or in combination with any other embodiment mentioned herein, the cosmetic composition comprises at least 1, 5, 10, 15, 20, 25, 30, 35, 40, 45, 50, 55, 60, 65, 70, 75, 80, 85, 90, 95, or 99 weight percent of at least one, two, three, four, or five cosmetic additives. Additionally, or in the alternative, the cosmetic composition can comprise less than 99, 95, 90, 85, 80, 75, 70, 65, 60, 55, or 50 weight percent of at least one, two, three, four, or five cosmetic additives. For example, the cosmetic composition can comprise 1 to 99, 1 to 95, 1 to 90, 1 to 85, 1 to 80, 5 to 99, 5 to 95, 5 to 90, 5 to 85, 10 to 99, 10 to 95, 10 to 85, 10 to 80, 15 to 99, 15 to 95, 15 to 90, 15 to 85, or 15 to 80 weight percent of at least one, two, three, four, or five cosmetic additives.

Generally, the cosmetic additives can include a solvent, a colorant, an oil, a wax, a fatty acid, an alcohol, an ester, a hydrocarbon, a silicone oil, a surfactant, a metal soap, a moisturizer, a thickener, a UV absorber, an antioxidant, an oil absorbent, an exfoliant, water, or a combination thereof.

In one embodiment or in combination with any other embodiment mentioned herein, the colorant comprises a pigment (e.g., an organic pigment and/or an inorganic pigment) and/or a dye.

In one embodiment or in combination with any other embodiment mentioned herein, the oil comprises triglycine, soybean oil, cocoa butter, palm oil, palm kernel oil, hardened oil, and/or hardened castor oil.

In one embodiment or in combination with any other embodiment mentioned herein, the wax comprises carnauba wax, candelilla wax, lanolin, lanolin, candelilla wax, cotton wax, Montan wax, Kapok wax, lanolin acetate, lanolin, and/or lanolin fatty acid isopropyl.

In one embodiment or in combination with any other embodiment mentioned herein, the fatty acid comprises lauric acid, myristic acid, palmitic acid, stearic acid, isostearic acid, behenic acid, oleic acid, undecylenic acid, linoleic acid, eicosapentaenoic acid (EPA), and/or docosahexaenoic acid.

In one embodiment or in combination with any other embodiment mentioned herein, the alcohol comprises cetyl alcohol, stearyl alcohol, isostearyl alcohol, 2-octyldodecanol, lauryl alcohol, behenyl alcohol, myristyl alcohol, oleyl alcohol, and/or cetostearyl alcohol.

In one embodiment or in combination with any other embodiment mentioned herein, the ester comprises isopropyl myristate, 2-octyldodecyl myristate, cetyl 2-ethylhexanoate, diisostearyl malate, tripropylene glycol dineopentate, isononyl isononanoate, isotorideyl isononanoate, cetyl octanoate, isocetyl palmitate, butyl stearate, hexyl laurate, myristyl myristate, decyl oleate, hexyl decyl dimethyloctanate, cetyl lactate, myristyl lactate, lanolin acetate, isosetyl stearate, isosetyl isostearate, cholesteryl 12-hydroxystearate, di-2-ethylhexanoic acid ethylene glycol, dipentaerythritol fatty acid ester, monoisostearate N-alkylglycol, dicaprate neopentyl glycol, di-2-heptylundecanoate glycerin, tri-2-ethylhexanoate trimethylpropane, Trimethylolpropane triisostearate, pentaerythritol tetra-2-ethylhexanoate, glycerin tri-2-ethylhexanoate, glycerin trioctanoate, glycerin triisopalmitate, trimethylolpropane triisostearate, ethylhexyl palmitate, glycerin trimyristate, tri-2-heptylundecanoic acid glyceride, castor oil fatty acid methyl ester, oleyl oleate, acetoglyceride, 2-heptylundecyl palmitate, diisobutyl adipate, N-lauroyl-L-Glutamic hexyldecyl palmitate, adipate hexyldecyl, diisopropyl sebacate, ethylhexyl succinate, and/or triethyl citrate.

In one embodiment or in combination with any other embodiment mentioned herein, the hydrocarbon comprises paraffin, petrolatum, and/or microcrystalline wax.

In one embodiment or in combination with any other embodiment mentioned herein, the surfactant comprises an anionic surfactant, a cationic surfactant, and/or a nonionic surfactant.

In one embodiment or in combination with any other embodiment mentioned herein, the thickener comprises guar gum, pectin, starch, gelatin, collagen, cellulosic derivatives, and/or mannan.

### EXPERIMENTS

The following experiments include preparation of cellulose esters, preparation of microparticles, preparation of cosmetic formulations containing the microparticles, and testing of these compositions.

### Preparation of Cellulose Esters

### Example 1-1

### a Cellulose Acetate Butyrate (CAB) was Prepared as Follows:

Cellulose and acid mixture [cellulose (4.3 parts) and acetic acid (AcOH) (11.8 parts)] was added to an agitated reactor and soaked unheated then, the mixture was heated to 55° C. Some amount of sulfuric acid was added and the reactor was cooled to 30° C. Following, a mixture of acetic anhydride (AC₂O) (8.9 parts) and butyric anhydride (Bu₂O) (5.6 parts) was added and the mixture cooled to around 9° C. with agitation. Additional sulfuric acid was added to a total of 0.6 parts and the resulting reaction mixture was warmed to 50° C. until the acylation reaction was complete and desired molecular weight is obtained. To this reaction mixture was added a mixture of butyric acid (BuOH) (18 parts) and H₂O (7.4 parts). The mixture was then stirred at 68° C. for 1020 min, but with the addition of a mixture of magnesium acetate (Mg(OAc)₂) (0.61 parts), BuOH (6.9 parts) and H₂O (2.7 parts) after 80 min. After the full length of time, the mixture was fully neutralized with a solution of Mg(OAc)₂ (0.86 parts), BuOH (1.5 parts) and H₂O (4.8 parts). The mixture was then precipitated in water, washed and dried by common methods to recover the CAB.

The degree of substitution (DS) and molecular weight (Mw) for the CAB of Example 1-1 was determined. The DS and Mw results are as follows:

### Example 1-1: Cellulose Acetate Butyrate (DS_{Ac}=1.87, DS_{Bu}=0.22, DS_{OH}=0.91, and M_{w}=90766)

### General Procedure for Preparation of Cellulose Esters Examples 1-2 to 1-8

A Cellulose mixture [cellulose and AcOH] (“Cellulose Mixture”) and sulfuric acid, AC₂O and Bu₂O (“Acylation Solution”) was cooled to 30° C. in an agitated reactor, and the reaction mixture cooled to around 7° C. with agitation. Additional sulfuric acid was added to a target amount, and the resulting reaction mixture, while stirring, was warmed to 45° C.-65° C. until the acylation reaction was complete and desired molecular weight is obtained. The reaction mixture treated with aqueous AcOH and BuOH (“Hydrolysis Solution”) and stirred at 68° C. under the Hydrolysis Conditions. Then, the reaction mixture was then quenched, neutralized, precipitated, washed and dried by common methods. The relative amounts and reaction conditions for Examples 1-2 to 1-8 are listed below in Table 1.1.

TABLE 1.1

| | Cellulose | | | | | | | | | Hydrolysis | |
|---|---|---|---|---|---|---|---|---|---|---|---|
| | Mixture | | Acylation Solution | | | | Hydrolysis Solution | | | Conditions | |
| | Cellulose | AcOH | H₂SO₄ | AcOH | AC₂O | Bu₂O | AcOH | BuOH | H₂O | Time | Temp |
| Ex | (parts) | (parts) | (parts) | (parts) | (parts) | (parts) | (parts) | (parts) | (parts) | (min) | (° C.) |
| 1-2* | 43 | 167.5 | 3.1 | 3.1 | 92 | 53 | 219 | 84 | 153.3 | 985 | 68 |
| | 43 | 167.5 | 3.1 | 3.1 | 92 | 53 | 219 | 84 | 153.3 | 966 | 68 |
| | 43 | 167.5 | 3.1 | 3.1 | 92 | 53 | 219 | 84 | 153.3 | 972 | 68 |
| | 43 | 167.5 | 3.1 | 3.1 | 92 | 53 | 219 | 84 | 153.3 | 989 | 68 |
| 1-3* | 43 | 167.5 | 3.1 | 3.1 | 89 | 56 | 184 | 84 | 146.2 | 1,020 | 68 |
| | 43 | 167.5 | 3.1 | 3.1 | 89 | 56 | 184 | 84 | 146.2 | 840 | 68 |
| 1-4 | 43 | 167.5 | 3.1 | 3.1 | 92 | 53 | 219 | 84 | 153.3 | 881 | 71 |
| 1-5 | 43 | 167.5 | 3.1 | 3.1 | 92 | 53 | 219 | 84 | 153.3 | 843 | 68 |
| 1-6 | 43 | 167.5 | 3.1 | 3.1 | 92 | 53 | 219 | 84 | 153.3 | 943 | 68 |
| 1-7* | 43 | 167.5 | 3.1 | 3.1 | 93 | 52 | 184 | 84 | 146.2 | 810 | 68 |
| | 43 | 167.5 | 3.1 | 3.1 | 93 | 52 | 184 | 84 | 146.2 | 960 | 68 |
| 1-8 | 43 | 167.5 | 3.1 | 3.1 | 89 | 56 | 184 | 84 | 146.2 | 840 | 68 |

*Blend of 2-4 CAB batches

### Characterization of the Cellulose Ester

The results showing DS and Mw for Examples 1-2 to 1-8 are listed in Table 1.2 below.

The DS and Mw were calculated as follows:

### Degree of Substitution

The degree of substitution for the substituents on the cellulose ester backbone is calculated using proton nuclear magnetic resonance spectroscopy. Gel permeation chromatography is performed on cellulose esters in stabilized tetrahydrofuran. The instrument is an Agilent 1260 which consists of a degasser, isocratic pump with a flow rate of 1.0 milliliters per minute, autosampler with an injection volume of 25 microliters, a column oven set at 28° C. and a refractive index detector at 28° C. The column set consists of an Agilent PLgel 5 micron guard, Mixed-C and Oligopore in series. The system is calibrated with monodisperse polystyrene standard ranging from approximately 4 million to 162 molecular weight. The sample is prepared by weighing approximately 25 milligrams of sample in 10 milliliters of solvent with the addition of 10 microliters of toluene to be used as a flow rate marker, add a stir bar into an 8-dram screw cap vial and stir until dissolution.

### Molecular Weight

The molecular weight is determined by gel permeation chromatography. Gel permeation chromatography is performed on cellulose esters in stabilized tetrahydrofuran. The instrument is an Agilent 1260 which consists of a degasser, isocratic pump with a flow rate of 1.0 milliliters per minute, autosampler with an injection volume of 25 microliters, a column oven set at 28° C. and a refractive index detector at 28° C. The column set consists of an Agilent PLgel 5 micron guard, Mixed-C and Oligopore in series. The system is calibrated with monodisperse polystyrene standard ranging from approximately 4 million to 162 molecular weight. The sample is prepared by weighing approximately 25 milligrams of sample in 10 milliliters of solvent with the addition of 10 microliters of toluene to be used as a flow rate marker, add a stir bar into an 8-dram screw cap vial and stir until dissolution.

### Preparation of Microparticles

### Examples 2-1 to 2-13

Microparticles were produced from the cellulose esters of examples 1-2 to 1-8. Examples 2-1 to 2-11 were made by jet milling and examples 2-12 and 2-13 was made by an emulsion process, as described below.

### Comparative Examples 1 to 3

Microparticles were also produced from commercial cellulose esters by jet milling, as described below.

The results for each of the microparticles, including which cellulose ester was used, the particle size (PS), oil absorption, and surface area (SSA/BET) are listed in Table 1.2 below.

### Jet Milling

There are multiple jet-milling configurations that can be used to reduce the size of particles. Such configurations are discussed in A. Chamayou and J. A. Dodds, Air Jet Milling, Handbook of Powder Technology, volume 12, Chapter 8, 2007 (“Chamayou”). FIG. 7 of Chamayou provides an example of a fluidized bed opposed jet mill that can be used to reduce the size of the cellulose ester particles described below in Table 1 (for the microparticles identified as being produced by jet milling). The jet-milling process was used to reduce cellulose ester particle size from 300-900 μm to ~10 μm. This basic jet mill operates as follows: The cellulose ester is placed into a hopper and introduced into the top of the mill (“FEED IN”) typically though a double valve arrangement (or through an injector). The cellulose ester particles fall by gravity to the bottom of the mill where they are swept up into one of three high pressure air streams that are geometrically oriented towards one another thus forming the so-called “pulverizing zone.” Within the pulverizing zone, the cellulose ester particles are size reduced via interparticle collisions. The size-reduced particles are then conveyed upwards by mass transport in the vertical airstream (fluidized bed) ultimately carrying them into the classifier. The classifier allows particles below the desired minimum size to be removed from the mill (“FINE OUT”). Particles that are above the maximum size are excluded from the classifier and returned to the fluidized bed eventually falling back down into the pulverizing zone for further size reduction. Particles that fall within the desired size range are ejected from the classifier into an appropriate product container. Many control parameters exist for optimizing productivity, particle size and particle size distribution shape, including but not necessarily limited to, classifier rotor speed, air nozzle pressure, and bed level.

### Emulsion Process

A clean, dry 3-liter kettle flask (with baffles) equipped with a two-tiered overhead mechanical agitator and bottom drop-out valve is prepared. The kettle is charged with 700-1,300 g deionized water, 6-13 g Tergitol 15-S-40, and 0-1.0 g PEG-100 stearate. Over 15 minutes, 7-12 g colloidal protector (low-, medium-, high-viscosity carboxymethylcellulose or methylcellulose) is added to the stirred aqueous mixture. The mixture is allowed to stir at ambient temperature for one hour or until homogeneous.

A separate clean, dry 2-liter kettle flask equipped with an overhead mechanical agitator and a bottom drop-out valve is prepared. A dope solution consisting of 10-100% cellulose ester is prepared using 0-5.0 parts C₁-C₄-alkyl acetate, 0-1.0 parts C₁-C₃ alcohol, and 0-0.80 parts deionized water (based on mass of the cellulose ester used). The mixture is allowed to stir at ambient temperature until homogeneous.

The dope solution is added to the 3-liter kettle over 30 minutes with vigorous stirring (250-500 rpm). Upon completion of the addition, the emulsion is recirculated through a flow cell containing a high-shear mixer (0-12,000 rpm) at the rate of 100-300 mL/minute for 45-60 minutes.

Upon completion of the high shear mixing time, the emulsion is then pumped at 300 mL/min into a 5-gallon bucket equipped with a single-tiered overhead mechanical agitator containing 4000-6000 g deionized water and agitated at 250-500 rpm for 0-16 hours. After the hold time, the volume is centrifuged to separate the spherical microparticles. The microparticles are suspended into 1-2 L of deionized water and centrifuged again. This washing process is repeated once.

The microparticles are placed in a sigma-bladed mixer and dried under vacuum (100-400 mmHg) at 50-100° C. for 16 hours. The recovered yield is about 75-85%. The typical/target particle size data is as follows: D (10)=3.07 μm; D (50)=8.20 μm; D (90)=22.6 μm; volume-based average particle size is about 11.3 μm.

### Microparticle Characterization

The microparticles were characterized by measuring particle size (PS), Oil Absorption and Surface Area (SSA/BET).

### Particle Size

Particle size distribution and mean/average particle size were determined by analyzing the microparticles with light dispersion using a Malvern Mastersizer 3000 (HydroMV dispersion unit) according to the following procedure:

### 1. Sample Preparation

a. Pour 1.5 ounces of isopropanol into a 2.5-ounce glass jar.
b. Scoop 0.5 grams of sample into the jar and shake vigorously

### 2. Instrument Initialization

a. Fill HydroMV dispersion unit completely with isopropanol and set stirrer speed to 3,500 RPM.
b. Allow isopropanol to circulate for about one minute, then open drain valve to release liquid into waste bucket.
c. Repeat steps a and b (dispersion unit needs to be rinsed twice).
d. Fill HydroMV dispersion unit halfway full with isopropanol and set stirrer speed to 3,500 RPM. Allow isopronol to circulate for 5 minutes, until energy on detectors has stabilizes.
e. Set stirrer speed to 3,000 RPM, then initialize instrument, and measure background.

### 3. Sample Measurement

a. Set obscuration rate between 2 and 5%. Set sample measurement time to 3 seconds for both red and blue light measurements
b. Add sample suspension dropwise until desired obscuration (~4%) rate is attained.
c. Measure sample.
d. After first measurement, sonicate the sample at 50% power for 120 seconds.
e. After sonication, wait until light energy stabilizes (usually less than one minute) then measure sample again.

### 4. Clean Up

a. Open drain valve to release liquid containing sample into waste bucket.
b. Repeat steps 2a thru 2c to rinse dispersion unit.
c. After all cellulose ester samples have been measured, rinse HydroMV dispersion unit twice with Millipore water.
d. Place lid onto HydroMV dispersion unit and close software.

The above procedure can be used to determine D10, D50, and D90 particle size, as well as D[4,3] mean particle size. Unless specified otherwise in the examples, particle size (PS) refers to D[4,3] mean particle size.

### Oil Absorption

Oil absorption was measured using an ASTM D281 standard test method to determine the oil absorption capacity of the tested microparticles. In this method a known amount of microparticles was weighed in a glass vial and olive oil was carefully added drop by drop to the absorbing microparticle with a plastic pipette. The microparticles were thoroughly mixed with oil after addition of every other droplet by rubbing with a sharp-edged steel spatula. The test is complete when exactly enough oil has been incorporated with the particle to produce very stiff putty-like paste which does not break and separate. The dropping bottle containing oil is accurately weighed. The oil absorption capability or uptake of the microparticles is calculated by the following equation: where A=initial weight of the dropping bottle with oil, B=final weight of the dropping bottle with oil, and W=weight of the starting microparticle sample in grams. Oil Absorption (g/g or g/100 g)=(A−B)/W

### SSA/BET

The surface area of the microparticles were measured using a gas absorption BET method according to ISO 9277 using a Micrometrics ASAP 2020 instrument.

The measurement was performed using the following procedure:

1) 0.5-1 gram samples are degassed at 60 C overnight. If degassing is not sufficient, temperature will raise 10 C higher, but lower than 100 C to avoid irreversible change of the surface.

2) Sample mass is collected by the weight difference of the empty sample tube and the sample tube filled with sample after degassing.

3) Krypton adsorption at 77K is used for the specific surface area analysis

4) Seven relative pressures from 0.06 to 0.20 are collected and fitted for BET specific surface area analysis.

The test results for PS, Oil Absorption and Surface Area for the microparticles tested are included in Table 1.2 below.

TABLE 1.2
Microparticles

| | | | | | | Oil | |
|---|---|---|---|---|---|---|---|
| | | CE | Mw | | PS | Absorption | SSA/BET |
| Example | CE used | Type | (k) | DS Levels | (microns) | (g/100 g) | (m2/g) |
| Ex 2-1 | Ex 1-2 | CAB | 45 | DS-OH: 0.87 | 11.2 | 108 | 3.18 |
| | | | | DS-Ac: 1.92 | | | |
| | | | | DS-Bu: 0.2 | | | |
| Ex 2-2 | Ex 1-3 | CAB | 81 | DS-OH: 0.9 | 9.7 | 76 | 4 |
| | | | | DS-Ac: 1.9 | | | |
| | | | | DS-Bu: 0.2 | | | |
| Ex 2-3 | Ex 1-4 | CAB | 73 | DS-OH: 0.93 | 7.88 | 89 | 3.17 |
| | | | | DS-Ac: 1.87 | | | |
| | | | | DS-Bu: 0.19 | | | |
| Ex 2-4 | Ex 1-4 | CAB | 73 | DS-OH: 0.93 | 9.75 | 82 | 4.27 |
| | | | | DS-Ac: 1.87 | | | |
| | | | | DS-Bu: 0.19 | | | |
| Ex 2-5 | Ex 1-4 | CAB | 73 | DS-OH: 0.93 | 14.4 | 80 | 2.93 |
| | | | | DS-Ac: 1.87 | | | |
| | | | | DS-Bu: 0.19 | | | |
| Ex 2-6 | Ex 1-5 | CAB | 18 | DS-OH: 0.8 | 6.2 | 79 | 3.31 |
| | | | | DS-Ac: 1.98 | | | |
| | | | | DS-Bu: 0.2 | | | |
| Ex 2-7 | Ex 1-5 | CAB | 18 | DS-OH: 0.8 | 9.3 | 69 | 2.54 |
| | | | | DS-Ac: 1.98 | | | |
| | | | | DS-Bu: 0.2 | | | |
| Ex 2-8 | Ex 1-5 | CAB | 18 | DS-OH: 0.8 | 14.3 | 81 | 2.05 |
| | | | | DS-Ac: 1.98 | | | |
| | | | | DS-Bu: 0.2 | | | |
| Ex 2-9 | Ex 1-6 | CAB | 14 | DS-OH: 0.8 | 5.2 | 75 | 3.59 |
| | | | | DS-Ac: 1.97 | | | |
| | | | | DS-Bu: 0.22 | | | |
| Ex 2-10 | Ex 1-6 | CAB | 14 | DS-OH: 0.8 | 10.6 | 76 | 2.42 |
| | | | | DS-Ac: 1.97 | | | |
| | | | | DS-Bu: 0.22 | | | |
| Ex 2-11 | Ex 1-6 | CAB | 14 | DS-OH: 0.8 | 14.8 | 77 | 1.82 |
| | | | | DS-Ac: 1.97 | | | |
| | | | | DS-Bu: 0.22 | | | |
| Ex 2-12 | Ex 1-7 | CAB | 76.7 | DS-OH: | 8.0 | 43 | 2.1 |
| | | | | 0.83:0.75 | | | |
| Ex 2-13 | Ex 1-8 | CAB | 72 | DS-OH: 0.8 | 11.2 | 79 | 1.67 |
| | | | | DS-Ac: 1.99 | | | |
| | | | | DS-Bu: 0.2 | | | |
| Comp. | CA 398-6 | CA | 55 | DS-OH: 0.6 | 10 | 108 | 6.53 |
| Ex 1 | | | | DS-Ac: 2.4 | | | |
| Comp. | CA 394- | CA | 115 | DS-OH: 0.6 | 10.9 | 113 | 13.84 |
| Ex 2 | 60S | | | DS-Ac: 2.4 | | | |
| Comp. | CA 394- | CA | 115 | DS-OH: 0.6 | 10.8 | 101 | 13.6 |
| Ex 3 | 60S* | | | DS-Ac: 2.4 | | | |

*Pre-ground in mechanical mill to 300 microns prior to jet milling

### Biodegradability Testing

Certain cellulose esters and microparticles from the examples above were tested to determine their biodegradability according to test method OECD 301B or OECD 301F.

### OECD 301B Test Description

A specific volume of a mineral medium is inoculated with a known concentration of the test substance, which serves as the nominal source of organic carbon. The medium is then aerated with carbon dioxide-free air at a controlled rate, either in the dark or under diffuse light conditions.

Over a period of 60 days, the degradation of the test substance is monitored by measuring the amount of carbon dioxide produced. The carbon dioxide is captured using barium or sodium hydroxide and quantified by titrating the remaining hydroxide or by measuring the inorganic carbon.

The quantity of carbon dioxide generated from the test substance, after adjusting for any carbon dioxide produced by the blank inoculum, is expressed as a percentage of the Theoretical Carbon Dioxide (ThCO2). Additionally, the extent of biodegradation can be calculated by analyzing the change in Dissolved Organic Carbon (DOC) levels at the beginning and end of the incubation period.

### OECD 301F Test Description

A measured volume of a mineral medium is inoculated with a known concentration of the test substance, which serves as nominal source of organic carbon. The medium is placed in a closed flask and stirred at a constant temperature (within a range of +1° C. or closer) for a maximum of 60 days.

The consumption of oxygen is determined using one of two methods: either by measuring the amount of oxygen required (produced electrolytically) to maintain a constant gas volume in the respirometer flask, or by monitoring changes in volume or pressure (or a combination of both) in the apparatus.

Any carbon dioxide produced during the process is absorbed using a solution of potassium hydroxide or another suitable absorbent. The amount of oxygen utilized by the microbial population during the biodegradation of the test substance is calculated by subtracting the oxygen uptake by the blank inoculum (which runs in parallel). This value is expressed as a percentage of Theoretical Oxygen Demand (ThOD) or, less ideally, Chemical Oxygen Demand (COD).

These tests were performed on cellulose ester examples 1-3, 1-4, 1-5, 1-6 and CA-398-6; and on microparticle examples 2-1, 2-2 and comparative Ex. 1. The results are listed below in Table 1.3.

TABLE 1.3
Biodegradability testing

| Example | Form | Test Method | Biodegradation (%) |
|---|---|---|---|
| Ex. 2-1 | Microparticles | OECD 301B | 83.4 |
| Ex. 2-2 | Microparticles | OECD 301F | 67.9 |
| Ex. 1-3 | CAB | OECD 301F | 60 |
| Ex. 1-4 | CAB | OECD 301F | 67 |
| Ex. 1-5 | CAB | OECD 301F | 62.7 |
| Ex. 1-6 | CAB | OECD 301F | 62.9 |
| Comp. Ex. 1 | Microparticles | OECD 301F | 76 |
| CA-398-6 | CA | OECD 301F | 32.5 |

A review of Table 1.3 reveals that the percent biodegradability is higher for the microparticles compared to the source cellulose ester used to make the microparticles and that Ex. 1-4 had the highest percent biodegradability of the cellulose esters tested.

### Cosmetic Formulations

Cosmetic formulations that were prepared and tested include a Water/Oil (W/O) Liquid Foundation, W/O Sunscreen, Lipstick and W/O Lotion.

### Water/Oil (W/O) Liquid Foundation

The ingredients used for preparing the W/O liquid foundation formulation are listed in Table 2.

TABLE 2
Ingredients of W/O Liquid Foundation

| | | Composition |
|---|---|---|
| Phase | Ingredients | (wt %) |
| A | Titanium Dioxide (and) Octyldodecanol (and) | 11.00% |
| | Jojoba Esters (and) Trihydroxystearin | |
| A | CI 77492 (colorant/pigment) | 1.20% |
| A | CI 77491 (colorant/pigment) | 0.20% |
| A | CI 77499 (colorant/pigment) | 0.17% |
| A | Polyhydroxystearic Acid | 1.00% |
| A | Coco caprylate/caprate | 2.00% |
| B | Isododecane | 6.00% |
| B | Squalane | 9.00% |
| B | Polyglyceryl-3 Diisostearate | 2.20% |
| B | Polyglyceryl-2 Dipolyhydroxystearate | 3.30% |
| B | Sucrose Acetate Isobutyrate (and) Caprylic/Capric | 3.00% |
| | Triglycerides | |
| B | Isododecane (and) Distearidimonium Hectorite | 4.00% |
| | (and) Propylene Carbonate | |
| C | DI Water | 42.43% |
| C | Magnesium Sulfate | 1.00% |
| C | propylene glycol | 2.00% |
| C | Glycerin | 1.50% |
| D | Capryloyl Glycerin/Sebacic Acid Copolymer | 4.00% |
| D | Phenoxyethanol (and) Ethylhexylglycerin | 1.00% |
| D | Microparticle | 5.00% |

### Preparation of W/O Liquid Foundation

The formulation for the W/O liquid foundation was prepared according to the following process:
1. Combine phase A (melt the polyhydroxy stearic acid first) and pass 3× through 3 roll mills;
2. Combine phase B and mix with overhead stirrer (large dissolver/400 RPM);
3. Add phase A to phase B and homogenize with Ultra Turax (5 k RPM) for 5 minutes;
4. Combine phase C and stir with magnetic stir bar until dissolved;
5. Slowly add phase C to phase A-B while mixing with Ultra Turax (10 k RPM);
6. Homogenize for 10 min at 10 k rpm;
7. Add ingredients of phase D, one by one, to the emulsion while mixing at 5 k RPM;
8. Add the microparticles while mixing with Ultra Turax at 10 k rpm.

### Testing

Testing of the W/O liquid foundation included optical effect testing and sensory panel evaluation.

Optical effects were assessed by using in-vitro skin-mimicking substrates in the form of synthetic textured leather with topographic patterns. Application procedures of formulation products on the substrates are the following.
1. Cut a large piece of synthetic leather into a small square shape with 5×5 cm² size by scissor;
2. Gently mix cosmetic formulation with a pipette or wooden stick;
3. Weigh total amount of 0.05 g of cosmetic formulation by analytical balance and drop evenly on 9 different spots on the textured leather substrate;
4. Rub and spread the product with a safety-gloved finger by circular motion for 1 min;
5. Allow the cosmetic product to dry for 5 min, visually assess samples (with ~45 degree angle) and give rating (1-5) for optical effects and coverage, making sure lighting condition is same for all evaluations;
6. Take pictures for record keeping purposes.

TABLE 3
Microparticles in W/O Liquid Foundation

| Microparticles | Optical Effect Rating (1-5) |
|---|---|
| No microparticles | 2.5 |
| 5 wt % Nylon-12 | 3.5 |
| 5 wt % Polymethyl Methacrylate | 3.5 |
| 5 wt % Ex. 2-2 | 4 |
| 5 wt % Cellulose | 3.25 |
| 5 wt % Cellulose Acetate | 3.75 |
| 5 wt % Boron Nitride | 3 |
| 5 wt % Silica | 3.75 |

Optical effect testing was also performed on commercial liquid foundation products. The results are shown below in Table 4.

TABLE 4
Microparticles in Commercial Products

| | | Optical Effect |
|---|---|---|
| Microparticles | Liquid Foundation Base | Rating (1-5) |
| None | Dior Forever Natural Nude | 3 |
| 5 wt % Ex. 2-2 | Dior Forever Natural Nude | 3.75 |
| None | bareMinerals Original | 3 |
| 5 wt % Ex. 2-2 | bareMinerals Original | 3.5 |
| None | NARS Light Reflecting | 3 |
| 5 wt % Ex. 2-2 | NARS Light Reflecting | 4 |

Sensory panel testing was performed by 5-10 panelists. The products were assigned randomly with numbers or letters to make blind tests. Small amount (10-30 mg) of products were applied on the back of the hand and then gently massaged with a finger-tip to spread by circular motion. Each person was asked to rate from 1 to 5 in the increasing order of intensity for each of the following sensory attributes:
Spreading: Evaluation of how well the sample spreads out and continues to provide consistent coverage when still wet and being applied onto the skin, with 1 being difficult to consistently spread out and 5 being very easy to consistently spread out;
Slipperiness: Evaluation of how slippery or easily movable (or gliding) the sample is when applying to the skin, with 1 being difficult to move and 5 being very easy to move;
Smoothness: Evaluation of how smooth the sample feels after it is applied and allowed to dry, with 1 being rough and 5 being very smooth;
Coverage: Evaluation of how uniform the color/pigmentation is after the sample is applied and allowed to dry, with 1 being non-uniform and 5 being highly uniform;
Soft focus: Evaluation of how diffuse the covered area looks and how well it optically blurs/hides blemishes/defects by manipulating light scattering to hide imperfections, with 1 being poor blurring/hiding ability and 5 being very good blurring/hiding effect;
Mattifying: Evaluation of how well the applied sample reduces shine (or gloss) and has less contrast with the skin, with 1 being shiny or glossy and a high contrast appearance and 5 being very flat (non-shiny) and low contrast appearance.

After each evaluation, the residual products on skin were removed by wipes and the next product was assessed. The results as average rating for each attribute are show below in Table 5.

TABLE 5
Particle size effect on sensory attributes: Rating 1-5

| | | | | Soft |
|---|---|---|---|---|
| | | | | focus/ |
| | Spreading | Slipperiness | Smoothness | Mattifying |
| W/O Liquid | 3 | 2.87 | 2.87 | 2.25 |
| Foundation | | | | |
| Base | | | | |
| 5 wt % Ex. 2-3 | 2.5 | 2.56 | 2.75 | 3.06 |
| 5 wt % Ex. 2-4 | 3.25 | 2.68 | 3.25 | 2.93 |
| 5 wt % Ex. 2-5 | 3.25 | 3.18 | 3.62 | 3.18 |

The shape of certain microparticles were identified and listed in Table 6 below. The effect of shape on the sensory testing was evaluated. The results are shown in Table 7 below.

TABLE 6
Shape: Spherical vs Spheroid

| Microparticles | INCI | Shape |
|---|---|---|
| Ex. 2-2 | Cellulose Acetate Butyrate | Non-spherical |
| | | (spheroid) |
| Comp. Ex. 2 | Cellulose Acetate | Non-spherical |
| | | (spheroid) |
| Ex. 2-12 | Cellulose Acetate Butyrate | Spherical |
| Ex. 2-13 | Cellulose Acetate Butyrate | Spherical |

TABLE 7
Shape effect on sensory attributes: Rating 1-5

| | | | | | Soft | |
|---|---|---|---|---|---|---|
| | | | | | Focus | |
| | Spread- | Slipper- | Smooth- | Cover- | (Blurr- | Matti- |
| | ing | iness | ness | age | ing) | fying |
| W/O | 3 | 3 | 3 | 3 | 3 | 3 |
| Liquid | | | | | | |
| Foundation | | | | | | |
| Base | | | | | | |
| 5 wt % | 3 | 2.71 | 3.14 | 3.85 | 4 | 3.91 |
| Ex. 2-2 | | | | | | |
| 5 wt % | 3.28 | 3.14 | 3.71 | 3.71 | 4.16 | 3.91 |
| Comp. | | | | | | |
| Ex. 2 | | | | | | |
| 5 wt % | 3 | 3.57 | 3.57 | 3.42 | 3.5 | 3.91 |
| Ex. 2-12 | | | | | | |
| 5 wt % | 3.28 | 3.14 | 3.57 | 2.85 | 3.5 | 3.75 |
| Ex. 2-13 | | | | | | |

A review of Tables 5 to 7 reveals that higher particle size generally provided higher ratings for spreading, slipperiness and smoothness, but had less effect on ratings for soft focus and mattifying effects (see Table 5), and spherical shape provided higher ratings for spreading, slipperiness and smoothness, but provided generally lower ratings for coverage, soft focus and mattifying effects (see Tables 6 and 7).

### Water/Oil (W/O) Sunscreen

The ingredients used for preparing the W/O Sunscreen formulation are listed in Table 8.

TABLE 8
Ingredients of W/O Sunscreen

| | | | Base | Microparticle | |
|---|---|---|---|---|---|
| Phase | Ingredient | Description | Formula | Formula | Source |
| A | Zinc Oxide | Zinc oxide, | 21.98 | 21.98 | Making |
| | Dispersion, | caprylic/capric | | | Cosmetics |
| | Paste | triglyceride, | | | |
| | | polyhydroxystearic | | | |
| | | acid, isostearic acid, | | | |
| | | lecithin, | | | |
| | | polyglyceryl-3 | | | |
| | | polyricinoleate | | | |
| B | Cetiol C5C | Coco caprylate | 10.66 | 10.66 | BASF |
| | | caprate | | | |
| B | CCT | Caprylic/Capric | 5.33 | 5.33 | Making |
| | | Triglyceride | | | Cosmetics |
| B | Emullium | Polyglyceryl-6 | 5.33 | 5.33 | Gattefossé |
| | Illustro | Polyhydroxystearate | | | |
| | | (and) Polyglyceryl-6 | | | |
| | | Polyricinoleate | | | |
| C | Demi water | Aqua | 49.24 | 46.24 | Demi |
| | | | | | water |
| C | Magnesium | Magnesium Sulfate | 2.13 | 2.13 | Merck |
| | Sulfate | | | | |
| C | Propanediol | Propanediol | 2.13 | 2.13 | Gova |
| C | Glycerin | Glycerin | 2.13 | 2.13 | Gova |
| D | Euxyl | Phenoxyethanol | 1.07 | 1.07 | Ashland |
| | PE9010 | (and) | | | |
| | | Ethylhexylglycerin | | | |
| E | Microparticle | | 0.00 | 3.00 | See |
| | | | | | Table 9 |
| | | TOTAL (Weight %) | 100.00 | 100.00 | |

### Preparation of W/O Sunscreen

The formulation for the W/O Sunscreen was prepared according to the following process:
1. Combine phase B (oil phase) and mix with overhead stirrer using a large dissolver at 400 RPM;
2. Heat oil phase to 50° C.;
3. Combine phase C (water phase) and stir with magnetic stir bar until mixed well;
4. Add Zinc Oxide dispersion (phase A) to oil phase;
5. Once phase A is mixed well into Phase B, transfer to a Silverson Mixer and mix at 5K RPM for 5 minutes;
6. Slowly add phase C-D to phase A-B while mixing with a Silverson (10K RPM);
7. Homogenize for 10 minutes at 10K RPM;
8. Post-add phase E (microparticles) while mixing with a Silverson at 10K RPM.

### Testing

Testing of the W/O Sunscreen included determining the sun protection factor (SPF) and light absorbance at different wavelengths. The SPF was determined in accordance with standard industry practice for measuring in vitro SPF. Additional formulations were prepared similar to above, but without including the zinc oxide dispersion paste (UV filter) in the formulation, to test light absorbance with and without the UV filter present. Light absorbance was measure using a spectrophotometer. The formulations tested and results are shown in Tables 9 and 10 below.

TABLE 9
In Vitro SPF Results

| Description | SPF |
|---|---|
| Base Sunscreen | 18 |
| Base with 3% Ex. 2-1 | 21 |
| Base with 3% silica (Solesphere H-53 from AGC Chemicals) | 26 |
| Base with 3% microcrystalline cellulose (SunSpheres Bio SPF | 31 |
| Booster from Dow Chemicals) | |
| Base with 3% Nylon-12 (SP-10 from Kobo) | 15 |
| Base with 3% PMMA (MSP-822 from Kobo) | 17 |

TABLE 10
Absorbance

| | Base | 3% Ex.2-1 | Base | 3% Ex. 2-1 |
|---|---|---|---|---|
| Sunscreen/ | without UV | without UV | with UV | with UV |
| Testing | filter | filter | filter | Filter |
| Wavelength | Absorbance | Absorbance | Absorbance | Absorbance |
| (nm) | | | | |
| 290 | 0.18 | 0.14 | 1.19 | 1.30 |
| 295 | 0.21 | 0.18 | 1.24 | 1.32 |
| 300 | 0.22 | 0.19 | 1.26 | 1.34 |
| 305 | 0.21 | 0.19 | 1.27 | 1.33 |
| 310 | 0.20 | 0.18 | 1.27 | 1.35 |
| 315 | 0.19 | 0.17 | 1.25 | 1.35 |
| 320 | 0.18 | 0.16 | 1.23 | 1.35 |
| 325 | 0.17 | 0.15 | 1.24 | 1.35 |
| 330 | 0.16 | 0.14 | 1.24 | 1.35 |
| 335 | 0.15 | 0.14 | 1.24 | 1.35 |
| 340 | 0.15 | 0.13 | 1.23 | 1.35 |
| 345 | 0.14 | 0.12 | 1.23 | 1.35 |
| 350 | 0.13 | 0.12 | 1.24 | 1.35 |
| 355 | 0.13 | 0.11 | 1.25 | 1.37 |
| 360 | 0.12 | 0.11 | 1.26 | 1.40 |
| 365 | 0.12 | 0.10 | 1.27 | 1.41 |
| 370 | 0.11 | 0.10 | 1.27 | 1.41 |
| 375 | 0.10 | 0.10 | 1.23 | 1.28 |
| 380 | 0.10 | 0.09 | 1.04 | 0.91 |
| 385 | 0.09 | 0.09 | 0.74 | 0.54 |
| 390 | 0.08 | 0.08 | 0.55 | 0.40 |
| 395 | 0.08 | 0.08 | 0.48 | 0.36 |
| 400 | 0.08 | 0.07 | 0.46 | 0.35 |

A review of Tables 9 and 10 reveals that the formulation with the Ex. 2-1 microparticles had higher SPF than formulations with Nylon-12 or PMMA and that including the Ex. 2-1 microparticles increased UV absorption when UV filters were present.

### Lipstick

The ingredients used for preparing the Lipstick formulation are listed in Table 11.

TABLE 11
Ingredients of Lipstick

| Phase | Ingredient | Description | Wt % |
|---|---|---|---|
| A | Cetiol SN | Cetearyl isononanoate | 27 |
| A | Castor Oil | Ricinus communis oil | 20 |
| A | Triglyceride | Caprylic/Capric Triglycerides | 16 |
| A | Ozokerite | Ozokerite | 11.6 |
| A | Carnauba Wax T1 | Copernicia Cerifera Cera | 4 |
| A | Beeswax | Cera Alba | 5 |
| B | SW50R40A | Synthetic Wax (and) Red 40 | 15 |
| | | Lake (and) Isopropyl Titanium | |
| | | Triisostearate | |
| C | Euxyl PE 9010 | Phenoxyethanol | 0.9 |
| C | Vitamin E DI-alpha | Tocopherol, Tocopherol Acetate | 0.5 |
| | tocopherol | | |

### Preparation of Lipstick

The formulation for the Lipstick was prepared according to the following process:

### Lipstick Base:

1. Heat Part A to 80° C. under propeller mixing.

2. Add part B. Mix for 10 minutes until uniform.

3. Add part C. Mix until uniform.

4. Let cool down.

### Incorporating Different Microparticles/Powder in the Lipstick Base:

1. Weigh the amount needed of lipstick base;
2. Heat to 80° C. under propeller mixing;
3. Add the microparticles to the melted lipstick base and mix for 10 minutes until uniform;
4. Pour the formulation into a lipstick mold and let cool down.

### Testing

Testing of the Lipstick included determining the ability of the lipstick to color a substrate by measuring the change in color as a function of microparticle/powder loading.

### Microparticles/Powders Tested:

Lipstick samples were made with loadings of 0, 1, 3, 6, 9% of microparticles/powders incorporated into the lipstick base. The microparticles/powders used are listed in Table 12.

TABLE 12
Microparticles/Powders used in Lipstick Testing

| Microparticle/Powder | Description |
|---|---|
| Ex. 2-1 | Cellulose Acetate Butyrate |
| KOBO SP-10 | Nylon-12 |
| KOBO MSP-822 | PMMA |
| Sunsphere H53 | Silica |
| KOBO Cellulobeads D10 | Cellulose |
| Tapioca starch | Starch |

### Color Testing Procedure:

The lipsticks are applied on a silicone substrate (to mimic the skin). A consistent application method on swatches of the substrate is used in order to compare different formulations. The L*a*b* color values of the colored swatches are measured with a handheld spectrophotometer (Konica Minolta 2600d). From these values, delta L* and delta a* are calculated with 0% loading being the starting value. A decreasing L* value reflects the swatches getting darker (or more intensely colored), an increasing delta a* value reflects a boost in red color (the pigment used for the lipstick base is a red40 lake). The evenness of the coverage is rated on a scale of 0 to 5 (5=best uniformity of color) by visually assessing the swatches. The results are shown in Tables 13 to 15 below.

TABLE 13
Delta L* as a Function of Loading

| | |
|---|---|
| Microparticle/ | Delta L* (% Loading) |

| Powder | 1 | 3 | 6 | 9 |
|---|---|---|---|---|
| Ex. 2-1 | −6.08 | −8.74 | −10.16 | −10.61 |
| KOBO SP-10 | −4.88 | −7.89 | −10.17 | −10.10 |
| KOBO MSP-822 | −3.05 | −5.43 | −7.96 | −8.13 |
| Sunsphere H53 | −4.09 | −5.99 | −6.98 | −7.39 |
| KOBO Cellulobeads | −5.46 | −10.24 | −12.08 | −14.13 |
| D10 | | | | |
| Tapioca starch | −3.54 | −4.54 | −9.56 | −8.71 |

A review of Table 13 reveals that addition of Ex. 2-1 microparticles results in a decrease in delta L* values, similar to Nylon-12, with both materials leading to a darker color of the applied lipstick (or a more intense color and desired effect). The best performance was reached at 6% loading. Ex. 2-1 performed better compared to Silica, PMMA and Starch on color intensity and the cellulose increased the color intensity the most.

TABLE 14
Delta a* as a Function of Loading

| | |
|---|---|
| Microparticle/ | Delta a* (% Loading) |

| Powder | 1 | 3 | 6 | 9 |
|---|---|---|---|---|
| Ex. 2-1 | 8.49 | 11.94 | 13.35 | 13.67 |
| KOBO SP-10 | 7.11 | 10.62 | 13.11 | 13.01 |
| KOBO MSP-822 | 4.67 | 7.59 | 10.95 | 10.49 |
| Sunsphere H53 | 5.83 | 8.39 | 9.55 | 10.20 |
| KOBO Cellulobeads | 8.13 | 13.35 | 15.05 | 16.94 |
| D10 | | | | |
| Tapioca starch | 4.90 | 6.29 | 12.13 | 9.89 |

A review of Table 14 reveals that addition of Ex. 2-1 microparticles results in an increase in red color (or increased delta a* values), similar to Nylon-12. The best performance was reached at 6% loading. Ex. 2-1 performed better compared to Silica, PMMA and Starch on red color boost and the cellulose increased the red color the most.

TABLE 15
Visual Assessment of Evenness of Coverage

| Loading | Ex. 2-1 | Nylon-12 | PMMA | Silica | Cellulose | Starch |
|---|---|---|---|---|---|---|
| 1% | 4 | 4 | 3 | 4 | 3 | 4 |
| 3% | 4 | 3 | 2 | 4 | 2 | 3 |
| 6% | 4 | 3 | 3 | 4 | 1 | 2 |
| 9% | 4 | 3 | 3 | 4 | 1 | 1 |

A review of Table 15 reveals that Ex. 2-1 and Silica showed the best distribution of pigment and the best evenness of coverage, followed by Nylon-12 and PMMA. Cellulose and Starch showed the worst evenness of coverage.

### W/O Lotion

The ingredients used for preparing a non-polar oil-based simplex W/O lotion formulation are listed in Table 16.

TABLE 16
Ingredients of W/O Lotion

| Phase | Trade Name | INCI | Wt % |
|---|---|---|---|
| A | Cetiol LC | Coco-caprylate/Caprate | 5.00% |
| A | Squalane | Squalane | 7.50% |
| A | Isododecane | Isododecane | 7.50% |
| A | Lameform TGI | Polyglyceryl-3 Diisostearate | 3.00% |
| A | Dehymuls | Polyglyceryl-2 Dipolyhydroxystearate | 2.00% |
| | PGPH | | |
| A | Bentone ISD V | Isododecane (and) Distearidimonium | 6.00% |
| | | Hectorite (and) Propylene Carbonate | |
| A | Euxyl PE9010 | Phenoxyethanol (and) | 1.00% |
| | | Ethylhexylglycerin | |
| B | DI Water | DI Water | 56.00% |
| B | Magnesium | Magnesium Sulfate | 1.00% |
| | Sulfate | | |
| B | Sodium | Sodium Chloride | 1.00% |
| | Chloride | | |
| B | Glycerin | Glycerin | 5.00% |
| E | **Beads** | Microbeads/Microparticles | 5.00% |

### Preparation of W/O Lotion

The formulation for the W/O lotion was prepared according to the following process:
1. Combine phase A and stir with overhead mixer (500 rpm) until homogeneous;
2. Combine phase B and stir until dissolved (using magnetic stir bar);
3. Add phase B to phase A while mixing with high shear (10 k rpm Ultra Turax mixer);
4. Homogenize for 5 minutes at 10 k rpm.

### Testing

Testing of the W/O lotion included gloss reduction testing. Gloss reduction was assessed by using a BYK micro-TRI gloss meter. Sample preparation and gloss reduction measurements were carried according to the following procedures.
1. Cut out a template to match the bottom of the gloss meter using painter's tape and adhere to bottom of meter prior to taking any measurements (to prevent seepage into parts of the meter);
2. Draw down a sample of the formulation on Leneta paper using a 4-mil squaredown bar by pulling the bar from top to bottom at constant speed/pressure while moving down the Leneta paper;
3. Measure gloss at three angles (20°, 60°, and 85°) initially and every 5 minutes thereafter for 30 minutes, as high-gloss surfaces with a gloss unit (GU) of 70 or higher should be measured using a 20° angle and semi-gloss surfaces with a GU range from 10-70 should be measured using a 60° angle;
4. After initial measurement, ensure that the gloss meter is placed back in the same location for subsequent measurements for accurate data, noting that the meter will make an imprint in the draw down film showing where the previous measurement was made;
5. Take three readings on each sample and obtain an average reading, while not moving the gloss meter, and perform 2 drawdowns per sample material;
6. For comparison and reporting, use the GU value at the 60° angle and report the data every 30 minutes.

Gloss reduction values are calculated with 0% loading (or base lotion) being the starting value and gloss reduction being reported as the percent change (reduction) in GU. The microparticles used in the test formulations and gloss reduction is shown in Table 17.

TABLE 17
Gloss Results for W/O Lotion

| Microparticles | Gloss Reduction (%) |
|---|---|
| Ex. 2-1 | 85 |
| Cellulose (and) Zinc Stearate | 54 |
| Nylon-12 | 31 |
| Boron Nitride | 16 |
| Silica | 83 |

A review of Table 17 reveals that the lotion formulation containing the Ex. 2-1 microparticles had the highest gloss reduction.

### Cellulose Ester Solubility Testing

CE dopes were prepared as shown in TABLE 18 below to determine the solubility of CE, such as cellulose acetate butyrate (CAB), Example 1-1, in a solvent system containing ethyl acetate (EA), n-propanol (nPrOH), and water. The preparation of Ex 3-1 is shown below.

### Preparation of CE Dopes

The CE dopes were prepared by charging a dry, 250 mL, 1-neck round-bottomed flask equipped with a magnetic stirrer with the respective amount of the solvent systems as shown in Table 18. The solvent systems were stirred, and the flask was then charged with the respective amount/type of CAB. The CAB was charged into the flask by slowly metering the solids at a rate such that the stirring vortex was able to move the solid particles into the solvent system without forming a large mass of powder at the top of the liquid phase. This mixture of solvent and CAB was stirred at room temperature for 45 minutes. If the mixture became homogeneous in this time period, the Dope was determined to be “soluble.” If the solid particles remained undissolved in the solvent system at 60 minutes, the Dope was determined to be “insoluble.”

In Dopes 3-1, 3-2, and 3-3, the solvent system contained ethyl acetate and n-propanol in varying amounts, but did not contain water. In Dopes 3-1a, 3-2a, and 3-3a, water was added to the solvent system. That is, the solvent systems of Dopes 3-1 and 3-1a, 3-2 and 3-2a, and 3-3 and 3-3a contained the same amount of ethyl acetate and n-propanol, and the only difference between the respective Dopes was the addition of water.

In Dope 3-4, the amount of CAB contained in the CE dope was increased to match its % CAB by mass to the % CAB by mass of the Dopes that did not contain water (i.e., Dopes 3-1, 3-2, and 3-3).

TABLE 18

| | EA | nPrOH | Water | CAB | EtOAc | nPrOH | Water | CAB | |
|---|---|---|---|---|---|---|---|---|---|
| Dope | (g) | (g) | (g) | (g) | (%) | (%) | (%) | (%) | Solubility |
| 3-1 | 80.0 | 5.0 | 0 | 15.0 | 80.0 | 5.0 | 0 | 15.0 | NO |
| 3-1a | 80.0 | 5.0 | 8.0 | 15.0 | 74.1 | 4.6 | 7.4 | 13.9 | NO |
| 3-2 | 75.0 | 10.0 | 0 | 15.0 | 75.0 | 10.0 | 0 | 15.0 | NO |
| 3-2a | 75.0 | 10.0 | 8.0 | 15.0 | 69.4 | 9.3 | 7.4 | 13.9 | YES |
| 3-3 | 70.0 | 15.0 | 0 | 15.0 | 70.0 | 15.0 | 0 | 15.0 | NO |
| 3-3a | 70.0 | 15.0 | 8.0 | 15.0 | 64.8 | 13.9 | 7.4 | 13.9 | YES |
| 3-4 | 70.0 | 15.0 | 8.0 | 16.2 | 64.8 | 13.9 | 7.4 | 15.0 | YES |

Notably, CE dopes that did not contain water were unable to solubilize the CE contained therein. For example, Dope 3-1 provided a chunky and non-homogeneous mixture, Dopes 3-2 and 3-3 initially provided stirrable slurries but became chunky and non-homogeneous after several minutes.

In comparison, Dope 3-1a provided a stirrable, rather than chunky, slurry. Dopes 3-2a and 3-3a provided homogeneous mixtures in which the CE was dissolved in the solvent system within 3 minutes.

As shown in Dope 3-4, increasing the mass of the CE contained in the CE dope also provided a homogenous mixture in which the CE was dissolved in the solvent system within 3 minutes. That is, increasing the % CAB of the CE dope did not appear to negatively impact the solubility of the CE in the solvent system.

1. A cosmetic composition comprising biodegradable microparticles, wherein the biodegradable microparticles comprise a mixed cellulose ester,
wherein the biodegradable microparticles exhibit at least 50 percent biodegradability at 60 days according to at least one of the OECD 301B, OECD 301C, or OECD 301F test methods,
wherein the mixed cellulose ester comprises—
(a) an average degree of substitution for acetyl substituents (“DS_{Ac}”) in the range of 0.1 to 2.3,
(b) an average degree of substitution for propionyl substituents (“DS_{Pr}”) or an average degree of substitution for butyryl substituents (“DS_{Bu}”) in the range of 0.1 to 0.5, and
(c) an average degree of substitution for hydroxyl substituents (“DS_{OH}”) in the range of 0.6 to 2.8.

2. The cosmetic composition according to claim 1, wherein the cosmetic composition comprises at least 0.5 weight percent of the biodegradable microparticles.

3. The cosmetic composition according to claim 1, wherein the biodegradable microparticles have an average sphericity less 50 percent,
wherein the biodegradable microparticles exhibit a monomodal particle size distribution with a span of at least 0.5 and/or less than 3.0, and
wherein the biodegradable microparticles have a D[4,3] particle size in the range of 1 to 50 microns.

4. The cosmetic composition according to claim 1, wherein the cosmetic composition is a foundation, a sunscreen, a lipstick, a mascara, an eye shadow, a lotion, a dry shampoo, a liquid shampoo, a body wash, a lotion, a hair conditioner, a skin moisturizer, a face wash, a tablet, a foot powder, a baby powder, a shaving cream, or a shaving gel.

5. The cosmetic composition according to claim 1, wherein the cosmetic composition comprises at least 1 and less than 60 weight percent of at least one cosmetic additive, wherein the at least one cosmetic additive comprises a colorant, an oil, a wax, a fatty acid, an alcohol, an ester, a hydrocarbon, a silicone oil, a surfactant, a metal soap, a moisturizer, a thickener, a UV absorber, an antioxidant, an oil absorbent, an exfoliant, water, or a combination thereof.

6. (canceled)

7. The cosmetic composition according to claim 1, wherein the biodegradable microparticles have a butyric acid content of less than 100 ppmw, and/or
wherein the biodegradable microparticles have an acetic acid content of less than 500 ppmw.

8. (canceled)

9. The cosmetic composition according to claim 1, wherein the mixed cellulose ester exhibits at least 4 percent biodegradability at 60 days according to at least one of the OECD 301B, OECD 301C, or OECD 301F test methods.

10. The cosmetic composition according to claim 1, wherein the DS_{Ac} is 1.5 to 2.3.

11. (canceled)

12. The cosmetic composition according to claim 1, wherein the DS_{Pr} is 0.1 to less than 0.5.

13. (canceled)

14. The cosmetic composition according to claim 1, wherein the DS_{Bu} is 0.1 to 0.3.

15. (canceled)

16. The cosmetic composition according to claim 1, wherein the DS_{OH} is 0.7 to less than 1.2.

17. (canceled)

18. The cosmetic composition according to claim 1, wherein the biodegradable microparticles comprise at least 75 weight percent of the mixed cellulose ester.

19. (canceled)

20. The cosmetic composition according to claim 1, wherein the biodegradable microparticles have a polydispersity index of less than 0.8 and a sphericity of at least 10 percent.

21. (canceled)

22. The cosmetic composition according to claim 1, wherein the biodegradable microparticles exhibit an oil absorption of at least 50 mL per 100 g, as measured using test method ASTM D281, wherein mineral oil is used instead of castor oil.

23. A cosmetic composition comprising 0.5 to 15 weight percent of biodegradable microparticles, wherein the biodegradable microparticles comprise a mixed cellulose ester,
wherein the biodegradable microparticles exhibit at least 50 percent biodegradability at 60 days according to at least one of the OECD 301B, OECD 301C, or OECD 301F test methods,
wherein the biodegradable microparticles have an average BET surface area in the range of 0.1 to 15 m²/g,
wherein the mixed cellulose ester comprises—
(a) an average degree of substitution for acetyl substituents (“DS_{Ac}”) in the range of 1.5 to 2.3,
(b) an average degree of substitution for butyryl substituents (“DS_{Bu}”) in the range of 0.1 to 0.3, and
(c) an average degree of substitution for hydroxyl substituents (“DS_{OH}”) in the range of 0.7 to 1.1.

24. The cosmetic composition according to claim 23, wherein the biodegradable microparticles consist essentially of the mixed cellulose ester.

25. (canceled)

26. A process for forming a cosmetic composition, the process comprising:
(a) providing a plurality of biodegradable microparticles comprising a mixed cellulose ester, wherein the biodegradable microparticles exhibit at least 50 percent biodegradability at 60 days according to at least one of the OECD 301B, OECD 301C, or OECD 301F test methods, wherein the mixed cellulose ester comprises—
(i) an average degree of substitution for acetyl substituents (“DS_{Ac}”) in the range of 1.5 to 2.3,
(ii) an average degree of substitution for propionyl substituents (“DS_{Pr}”) or an average degree of substitution for butyryl substituents (“DS_{Bu}”) in the range of 0.1 to 0.3, and
(iii) an average degree of substitution for hydroxyl substituents (“DS_{OH}”) in the range of 0.7 to 1.1; and
(b) combining the biodegradable microparticles with one or more cosmetic additives to thereby form a pre-cosmetic mixture; and
(c) forming the cosmetic composition from the pre-cosmetic mixture,
wherein the cosmetic composition comprises at least 2 weight percent of the biodegradable microparticles.

27. The process according to claim 26, wherein the biodegradable microparticles are produced by jet milling and have an average sphericity in the range from 5 to 30 percent.

28. The process according to claim 27, wherein the cosmetic composition is a foundation, a sunscreen, a lipstick, a mascara, an eye shadow, a lotion, a dry shampoo, a liquid shampoo, a body wash, a lotion, a hair conditioner, a skin moisturizer, a face wash, a tablet, a foot powder, a baby powder, a shaving cream, or a shaving gel.

29. The process according to claim 28, wherein the biodegradable microparticles have a D[4,3] mean particle size in the range of 1 to 10 microns.

30. (canceled)
